SEQUENCE LISTING (81 sequences)

SEQ ID NO: 1 - protein, 141 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFHVMGALAEMERELILERVMAGIAAARNKGRRWGRPP

SEQ ID NO: 2 - protein, 141 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELILERVMAGIAAARNKGRRWGRPP

SEQ ID NO: 3 - protein, 141 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELILERVMAGIAAARNKGRRWGRPP

SEQ ID NO: 4 - protein, 355 aa, Artificial Sequence
  Description of Artificial Sequence Synthetic polypeptide
MDYKDHDGDYKDHDIDYKDDDDKAPKKKRKVDMLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELILERVMAGIAAARNKGRRWGRPPKSGTGERPFQCRICMRNFSRPYTLRLHIRTHTGEKPFACDICGRKFADNSNRIKHTKIHTHPRAPIPKPFQCRICMRNFSQSSDLSRHIRTHTGEKPFACDICGRKFAQSGNLARHTKIHTGSQKPFQCRICMRNFSRQDCLSLHIRTHTGEKPFACDICGRKFARNDNRKTHTKIHLRQKD

SEQ ID NO: 5 - protein, 359 aa, Artificial Sequence
  Description of Artificial Sequence Synthetic polypeptide
MDYKDHDGDYKDHDIDYKDDDDKAPKKKRKVDMLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELILERVMAGIAAARNKGRRWGRPPKSGTGERPFQCRICMRNFSLRHHLTRHIRTHTGEKPFACDICGRKFAQSYARTLHTKIHTHPRAPIPKPFQCRICMRNFSWRSSLKTHIRTHTGEKPFACDICGRKFADRSNRKTHTKIHTHPRAPIPKPFQCRICMRNFSDRSNLSRHIRTHTGEKPFACDICGRKFAQSGNLARHTKIHLRQKD

SEQ ID NO: 6 - DNA, 468 bp, Artificial Sequence
  Description of Artificial Sequence Synthetic polynucleotide
atgctgatcggctatgtgcgggtgtccaccaacgaccagaacaccgacctgcagagaaacgcccttgtgtgtgccggatgcgagcagatcttcgaggataagctgagcggcaccagaaccgacagacccggactgaagagagccctgaagagactgcagaaaggcgacaccctggtcgtgtggaagctggatagactgggccgcagcatgaagcacctgatcagccttgtgggcgagctgagagagcggggcatcaactttagaagcctgaccgacagcatcgacacaagcagccctatgggcagattcttcttctacgtgatgggcgccctggccgagatggaaagagagctgatcctggagagagtcatggccggaatcgccgctgccagaaacaagggaagaagatggggccggccacctaagtctggcacaggcgagaggcccttccagtgtcgaatctgcatg

SEQ ID NO: 7 - DNA, 363 bp, Artificial Sequence
  Description of Artificial Sequence Synthetic polynucleotide
atgctgatcggctatgtgcgggtgtccaccaacgaccagaacaccgacctgcagagaaacgcccttgtgtgtgccggatgcgagcagatcttcgaggataagctgagcggcaccagaaccgacagacccggactgaagagagccctgaagagactgcagaaaggcgacaccctggtcgtgtggaagctggatagactgggccgcagcatgaagcacctgatcagccttgtgggcgagctgagagagcggggcatcaactttagaagcctgaccgacagcatcgacacaagcagccctatgggcagattcaacttccatgtgatgggcgccctggccgagatgagaagatggggccggccacct

SEQ ID NO: 8 - protein, 5 aa, Artificial Sequence
  Description of Artificial Sequence Synthetic peptide
KSGTG

SEQ ID NO: 9 - protein, 177 aa, Artificial Sequence
  Description of Artificial Sequence Synthetic polypeptide
ERPFQCRICMRNFSRPYTLRLHIRTHTGEKPFACDICGRKFADNSNRIKHTKIHTHPRAPIPKPFQCRICMRNFSQSSDLSRHIRTHTGEKPFACDICGRKFAQSGNLARHTKIHTGSQKPFQCRICMRNFSRQDCLSLHIRTHTGEKPFACDICGRKFARNDNRKTHTKIHLRQKD

SEQ ID NO: 10 - protein, 181 aa, Artificial Sequence
  Description of Artificial Sequence Synthetic polypeptide
ERPFQCRICMRNFSLRHHLTRHIRTHTGEKPFACDICGRKFAQSYARTLHTKIHTHPRAPIPKPFQCRICMRNFSWRSSLKTHIRTHTGEKPFACDICGRKFADRSNRKTHTKIHTHPRAPIPKPFQCRICMRNFSDRSNLSRHIRTHTGEKPFACDICGRKFAQSGNLARHTKIHLRQKD

SEQ ID NO: 11 - DNA, 2947 bp, Artificial Sequence
  Description of Artificial Sequence Synthetic polynucleotide
agttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatgaaaaggtggaagctttacttggttataactttacttttaatacctagaacagtgagtcttcaaactgctttattgatgcccaatttataaaaagtttcctgagcatttacccctaatatatgctctgctaatggagctatgcaatctactgaatgtcataatcaacttcaaggatatactgaagctatgtgggcttctcatgaaagagaattttttgcttgtaaggaaagatgtatgcttgaaataagccaccatagagacccatgtcctaacagctccctggagacggctaatgatacttggaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctc

SEQ ID NO: 12 - DNA, 20 bp, Artificial Sequence
  Description of Artificial Sequence Synthetic oligonucleotide
gttataactttacttttaat

SEQ ID NO: 13 - DNA, 19 bp, Artificial Sequence
  Description of Artificial Sequence Synthetic oligonucleotide
aaggtggaagctttacttg

SEQ ID NO: 14 - DNA, 20 bp, Artificial Sequence
  Description of Artificial Sequence Synthetic oligonucleotide
acctagaacagtgagtcttc

SEQ ID NO: 15 - DNA, 59 bp, Unknown
  Description of Unknown Zinc-finger recombinase target sequence
aaggtggaagctttacttggttataactttacttttaatacctagaacagtgagtcttc

SEQ ID NO: 16 - DNA, 3934 bp, Artificial Sequence
  Description of Artificial Sequence Synthetic polynucleotide
ctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagagccaagctgactagcgtttaaacttaagctgatccactagtccagtgtggtggaattcgccaccatggactacaaagaccacgacggtgattataaagatcacgacatcgattacaaggacgacgacgacaaggcccccaagaagaagaggaaggtcgacatgctgatcggctatgtgcgggtgtccaccaacgaccagaacaccgacctgcagagaaacgcccttgtgtgtgccggatgcgagcagatcttcgaggataagctgagcggcaccagaaccgacagacccggactgaagagagccctgaagagactgcagaaaggcgacaccctggtcgtgtggaagctggatagactgggccgcagcatgaagcacctgatcagccttgtgggcgagctgagagagcggggcatcaactttagaagcctgaccgacagcatcgacacaagcagccctatgggcagattcaacttccatgtgatgggcgccctggccgagatggaaagagagctgatcctggagagagtcatggccggaatcgccgctgccagaaacaagggaagaagatggggccggccacctaagtctggcacaggcgagaggcccttccagtgtcgaatctgcatgcgtaacttcagtcgcccgtacaccctgcgcctgcacatccgcacccacaccggcgagaagccttttgcctgtgacatttgtgggaggaaatttgccgacaactccaaccgcatcaagcataccaagatacacacgcatcccagggcacctattcccaagcccttccagtgtcgaatctgcatgcgtaacttcagtcagtcctccgacctgtcccgccacatccgcacccacaccggcgagaagccttttgcctgtgacatttgtgggaggaaatttgcccagtccggcaacctggcccgccataccaagatacacacgggatctcagaagcccttccagtgtcgaatctgcatgcgtaacttcagtcgccaggactgtctgtccctgcacatccgcacccacaccggcgagaagccttttgcctgtgacatttgtgggaggaaatttgcccgcaacgacaaccgcaagacccataccaagatacacctgcggcagaaggactgataac

SEQ ID NO: 17 - DNA, 3833 bp, Artificial Sequence
  Description of Artificial Sequence Synthetic polynucleotide
ggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagagccaagctgactagcgtttaaacttaagctgatccactagtccagtgtggtggaattcgccaccatggactacaaagaccacgacggtgattataaagatcacgacatcgattacaaggacgacgacgacaaggcccccaagaagaagaggaaggtcgacatgctgatcggctatgtgcgggtgtccaccaacgaccagaacaccgacctgcagagaaacgcccttgtgtgtgccggatgcgagcagatcttcgaggataagctgagcggcaccagaaccgacagacccggactgaagagagccctgaagagactgcagaaaggcgacaccctggtcgtgtggaagctggatagactgggccgcagcatgaagcacctgatcagccttgtgggcgagctgagagagcggggcatcaactttagaagcctgaccgacagcatcgacacaagcagccctatgggcagattcaacttccatgtgatgggcgccctggccgagatggaaagagagctgatcctggagagagtcatggccggaatcgccgctgccagaaacaagggaagaagatggggccggccacctaagtctggcacaggcgagaggcccttccagtgtcgaatctgcatgcgtaacttcagtctgcgccaccacctgacccgccacatccgcacccacaccggcgagaagccttttgcctgtgacatttgtgggaggaaatttgcccagtcctacgcccgcaccctgcataccaagatacacacgcatcccagggcacctattcccaagcccttccagtgtcgaatctgcatgcgtaacttcagttggcgctcctccctgaagacccacatccgcacccacaccggcgagaagccttttgcctgtgacatttgtgggaggaaatttgccgaccgctccaaccgcaagacccataccaagatacacacgcacccgcgcgccccgatcccgaagcccttccagtgtcgaatctgcatgcgtaacttcagtgaccgctccaacctgtcccgccacatccgcacccacaccggcgagaagccttttgcctgtgacatttgtgggaggaaatttgcccagtccggcaacctggcccgccataccaagatacacctgcggcagaaggactgataac

SEQ ID NO: 18 - protein, 22 aa, Artificial Sequence
  Description of Artificial Sequence Synthetic peptide
DYKDHDGDYKDHDIDYKDDDDK

SEQ ID NO: 19 - protein, 7 aa, Simian virus 40
PKKKRKV

SEQ ID NO: 20 - DNA, 170 bp, Artificial Sequence
  Description of Artificial Sequence Synthetic polynucleotide
taaaacgacggccagtgaattcatgaaaaggtggaagctttacttggttataactttacttttaatacctagaacagtgagtcttcaaactgctttattgatgcccaatttataaaaagtttcctgagcatttacccctaatatatgctctgctaatggagctatgcaat

SEQ ID NO: 21 - DNA, 44 bp, Artificial Sequence
  Description of Artificial Sequence Synthetic primer
tgctaatggagctatgcaatctactgaatgtcataatcaacttc

SEQ ID NO: 22 - DNA, 41 bp, Artificial Sequence
  Description of Artificial Sequence Synthetic primer
attcactggccgtcgttttacaacgtcgtgactgggaaaac

SEQ ID NO: 23 - DNA, 9 bp, Artificial Sequence
  Description of Artificial Sequence Synthetic oligonucleotide
tgtatttgc

SEQ ID NO: 24 - DNA, 9 bp, Artificial Sequence
  Description of Artificial Sequence Synthetic oligonucleotide
gctttattg

SEQ ID NO: 25 - DNA, 20 bp, Artificial Sequence
  Description of Artificial Sequence Synthetic primer
acacgacgctcttccgatct

SEQ ID NO: 26 - DNA, 18 bp, Artificial Sequence
  Description of Artificial Sequence Synthetic primer
gacgtgtgctcttccgat

SEQ ID NO: 27 - DNA, 20 bp, Artificial Sequence
  Description of Artificial Sequence Synthetic oligonucleotide
ctaattaccttcattatagt

SEQ ID NO: 28 - DNA, 20 bp, Artificial Sequence
  Description of Artificial Sequence Synthetic oligonucleotide
acaaatagtataatttgagt

SEQ ID NO: 29 - DNA, 22 bp, Artificial Sequence
  Description of Artificial Sequence Synthetic primer
taaaacgacggccagtgaattc

SEQ ID NO: 30 - DNA, 22 bp, Artificial Sequence
  Description of Artificial Sequence Synthetic primer
tctgctaatggagctatgcaat

SEQ ID NO: 31 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFHVMGALAEMERELIIERTMAGLAAARNKGRIGGRPPKSG

SEQ ID NO: 32 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFHVMGALAEMERELIIERTMAGIAAARNKGRRFGRPPKSG

SEQ ID NO: 33 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFHVMGALAEMERELIIERVMAGLAAARNKGRRFGRPPKSG

SEQ ID NO: 34 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFHVMGALAEMERLSILERPMAGHAAARNKGRRFGRPPKSG

SEQ ID NO: 35 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFHVMGALAEMERELIIERTSAGRAAAINKGRIMGRPRKSG

SEQ ID NO: 36 - protein, 141 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFYVMGALAEMERELILERVMAGIAAARNKGRRWGRPP

SEQ ID NO: 37 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFYVMGALAEMERELIIERTMAGLAAARNKGRIGGRPPKSG

SEQ ID NO: 38 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFYVMGALAEMERELIIERTMAGIAAARNKGRRFGRPPKSG

SEQ ID NO: 39 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFYVMGALAEMERELIIERVMAGLAAARNKGRRFGRPPKSG

SEQ ID NO: 40 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERLSILERPMAGHAAARNKGRRFGRPPKSG

SEQ ID NO: 41 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERTSAGRAAAINKGRIMGRPRKSG

SEQ ID NO: 42 - protein, 141 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFNFHVMGALAEMERELILERVMAGIAAARNKGRRWGRPP

SEQ ID NO: 43 - protein, 141 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFFFHVMGALAEMERELILERVMAGIAAARNKGRRWGRPP

SEQ ID NO: 44 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFNFHVMGALAEMERELIIERTMAGLAAARNKGRIGGRPPKSG

SEQ ID NO: 45 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELIIERTMAGLAAARNKGRIGGRPPKSG

SEQ ID NO: 46 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERTMAGLAAARNKGRIGGRPPKSG

SEQ ID NO: 47 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFFFHVMGALAEMERELIIERTMAGLAAARNKGRIGGRPPKSG

SEQ ID NO: 48 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFNFHVMGALAEMERELIIERTMAGIAAARNKGRRFGRPPKSG

SEQ ID NO: 49 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELIIERTMAGIAAARNKGRRFGRPPKSG

SEQ ID NO: 50 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERTMAGIAAARNKGRRFGRPPKSG

SEQ ID NO: 51 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFFFHVMGALAEMERELIIERTMAGIAAARNKGRRFGRPPKSG

SEQ ID NO: 52 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFNFHVMGALAEMERELIIERVMAGLAAARNKGRRFGRPPKSG

SEQ ID NO: 53 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELIIERVMAGLAAARNKGRRFGRPPKSG

SEQ ID NO: 54 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFFFYVMGALAEMERELIIERVMAGLAAARNKGRRFGRPPKSG

SEQ ID NO: 55 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFFFHVMGALAEMERELIIERVMAGLAAARNKGRRFGRPPKSG

SEQ ID NO: 56 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFYVMGALAEMERLSILERPMAGHAAARNKGRRFGRPPKSG

SEQ ID NO: 57 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFNFHVMGALAEMERLSILERPMAGHAAARNKGRRFGRPPKSG

SEQ ID NO: 58 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERLSILERPMAGHAAARNKGRRFGRPPKSG

SEQ ID NO: 59 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFFFHVMGALAEMERLSILERPMAGHAAARNKGRRFGRPPKSG

SEQ ID NO: 60 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFYVMGALAEMERELIIERTSAGRAAAINKGRIMGRPRKSG

SEQ ID NO: 61 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFNFHVMGALAEMERELIIERTSAGRAAAINKGRIMGRPRKSG

SEQ ID NO: 62 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELIIERTSAGRAAAINKGRIMGRPRKSG

SEQ ID NO: 63 - protein, 144 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSVDTSSPMGRFFFHVMGALAEMERELIIERTSAGRAAAINKGRIMGRPRKSG

SEQ ID NO: 64 - protein, 323 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELILERVMAGIAAARNKGRRWGRPPKSGTGERPFQCRICMRNFSRPYTLRLHIRTHTGEKPFACDICGRKFADNSNRIKHTKIHTHPRAPIPKPFQCRICMRNFSQSSDLSRHIRTHTGEKPFACDICGRKFAQSGNLARHTKIHTGSQKPFQCRICMRNFSRQDCLSLHIRTHTGEKPFACDICGRKFARNDNRKTHTKIHLRQKD

SEQ ID NO: 65 - protein, 327 aa, Artificial Sequence
  Description of Artificial Sequence Synthetic polypeptide
MLIGYVRVSTNDQNTDLQRNALVCAGCEQIFEDKLSGTRTDRPGLKRALKRLQKGDTLVVWKLDRLGRSMKHLISLVGELRERGINFRSLTDSIDTSSPMGRFNFHVMGALAEMERELILERVMAGIAAARNKGRRWGRPPKSGTGERPFQCRICMRNFSLRHHLTRHIRTHTGEKPFACDICGRKFAQSYARTLHTKIHTHPRAPIPKPFQCRICMRNFSWRSSLKTHIRTHTGEKPFACDICGRKFADRSNRKTHTKIHTHPRAPIPKPFQCRICMRNFSDRSNLSRHIRTHTGEKPFACDICGRKFAQSGNLARHTKIHLRQKD

SEQ ID NO: 66 - protein, 7 aa, Artificial Sequence
  Description of Artificial Sequence Synthetic peptide
LRHHLTR

SEQ ID NO: 67 - protein, 7 aa, Artificial Sequence
  Description of Artificial Sequence Synthetic peptide
DRSTLRQ

SEQ ID NO: 68 - protein, 7 aa, Artificial Sequence
  Description of Artificial Sequence Synthetic peptide
QSANRTK

SEQ ID NO: 69 - protein, 7 aa, Artificial Sequence
  Description of Artificial Sequence Synthetic peptide
DNSYLPR

SEQ ID NO: 70 - protein, 7 aa, Artificial Sequence
  Description of Artificial Sequence Synthetic peptide
RSDNLSV

SEQ ID NO: 71 - protein, 7 aa, Artificial Sequence
  Description of Artificial Sequence Synthetic peptide
QKATRIN

SEQ ID NO: 72 - protein, 7 aa, Artificial Sequence
  Description of Artificial Sequence Synthetic peptide
RSDNLAR

SEQ ID NO: 73 - protein, 7 aa, Artificial Sequence
  Description of Artificial Sequence Synthetic peptide
LRQHLRA

SEQ ID NO: 74 - protein, 7 aa, Artificial Sequence
  Description of Artificial Sequence Synthetic peptide
QSGNLAR

SEQ ID NO: 75 - protein, 7 aa, Artificial Sequence
  Description of Artificial Sequence Synthetic peptide
QSTPRTT

SEQ ID NO: 76 - protein, 7 aa, Artificial Sequence
  Description of Artificial Sequence Synthetic peptide
DRSNRIK

SEQ ID NO: 77 - DNA, 18 bp, Unknown
  Description of Unknown HPRT-specific zinc finger nuclease target sequence
gtaaagttataaccaagt

SEQ ID NO: 78 - DNA, 16 bp, Unknown
  Description of Unknown HPRT-specific zinc finger nuclease target sequence
tacctagaacagtgag

SEQ ID NO: 79 - DNA, 3511 bp, Artificial Sequence
  Description of Artificial Sequence Synthetic polynucleotide
gccccactgtggggtggaggggatcgacacaaaacgacaatacaagaatctcgcaaaccggccctgggaatataaggctcagaccatccttccagactcaggagatcatgtgttgccgaccgtccttccgtcaagcgggttttagagattcaaggtcgagaagcaaaagcagcaggagatgcttctctgcatccgtcttaaactgctttgagacatgaaaaggtggaagctttacttggttataactttacttttaatacctagaacagtgagtcttcaaactgctttattgatgcccaatttataaaaagtttcctgaggcggactggacgtaaaacattctgctaatggagctatgcaatctactgaatgtcataatcaacttcaaggatatactgaagctatgtgggcttctcatgaaagagaattttttgcttgtaaggaaagatgtatgcttgaaataagccaccatagagacccatgtcctaacagctccctggagacggctaatgatacttggaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatgaaaaggtggaagctttacttggttataactttacttttaatacctagaacagtgagtcttcaaacttgtatttgcatgcccaatttataaaaagtttcctgaggcggactggacgtaaaacatgagcaacgaccacaacacacccacagggacaaacaccgcccaagtcaaagatcactcatgtcgtcgaggaagtggaggttgcttacatggctccttctctggacagtgtgtcaccagataaggaatctgcctaacaggaggtgggaccgttcgagaatatcaggagactaggaaggaggaggcctaaggatggggcttttctgtcaccaatcctgtccctagtg

SEQ ID NO: 80 - DNA, 44 bp, Artificial Sequence
  Description of Artificial Sequence Synthetic primer
  modified_base (21)..(24): a, c, t, g, unknown or other
acacgacgctcttccgatctnnnnatgcttctctgcatccgtct

SEQ ID NO: 81 - DNA, 40 bp, Artificial Sequence
  Description of Artificial Sequence Synthetic primer
gacgtgtgctcttccgatctatgttttacgtccagtccgc

(19) United States
(12) Patent Application Publication
(10) Pub. No.: US 2021/0130828 A1
(43) Pub. Date: May 6, 2021

(54) GIN RECOMBINASE VARIANTS

(71) Applicant: SANGAMO THERAPEUTICS, INC., Richmond, CA (US)

(72) Inventors: Friedrich Alexander Fauser, Fremont, CA (US); Jeffrey Christopher Miller, San Rafael, CA (US)

(21) Appl. No.: 17/085,319

(22) Filed: Oct. 30, 2020

Related U.S. Application Data
(60) Provisional application No. 62/929,563, filed on Nov. 1, 2019.

(51) Int. Cl. C12N 15/113; C12N 15/63

(52) U.S. Cl. CPC C12N 15/113; C12N 15/63

(57) ABSTRACT

The present disclosure provides a Gin recombinase catalytic domain variant and a zinc finger recombinase comprising a Gin recombinase catalytic domain variant operatively linked to a zinc finger nucleotide binding domain and methods for modifying the genome of a cell or to treat a disorder in a subject by using said zinc finger recombinase protein.

### CROSS-REFERENCE TO RELATED APPLICATIONS

This application claims priority and benefit from U.S. Provisional Application No. 62/929,563, filed Nov. 1, 2019, the contents of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 30, 2020, is named 000222-0006-101-SL.txt and is 93,563 bytes in size.

### TECHNICAL FIELD

The present disclosure is in the field of serine recombinases and methods for gene editing.

### BACKGROUND

Targeted genetic engineering is driving progress in new areas of basic biological research, biotechnology, and gene therapy. Site-specific endonucleases, including zinc-finger recombinases (ZFNs), meganucleases, TAL effector nucleases (TALENs), and CRISPR/Cas systems, have dramatically enhanced the speed and efficiency with which researchers can introduce targeted genetic modifications into cells and organisms.

Although site-specific nucleases are versatile and promote a broad range of genetic alterations, they rely on cellular DNA repair mechanisms, such as error-prone non-homologous end joining (NHEJ) and homology-directed repair (HDR), to induce custom alterations. The lack of availability of DNA repair pathways within certain cell types, however, may reduce the utility of this technology. In particular, poor induction of HDR via nuclease-induced DNA double-strand breaks (DSBs) or nicks has been shown to be a major limiting factor for achieving high rates of site-specific integration.

Site-specific recombinases (SSRs, e.g., Cre, Flp, phiC31, and Bxbl) represent a versatile alternative to conventional site-specific nucleases for targeted genetic engineering. SSRs are highly specialized enzymes that promote high-fidelity DNA rearrangements (e.g., integration, excision, or inversion) between defined segments of DNA. The strict target specificities demonstrated by many SSR systems, however, have limited their adoption in disciplines that require tools with highly flexible recognition capabilities.

Hybrid recombinases composed of catalytic domains derived from the resolvase/invertase family of serine recombinases (e.g., Gin, Hin, Tn3, and γδ) fused to custom-designed Zinc-Finger or TAL effector DNA-binding domains or CRISPR/Cas systems represent a promising tool for genetic engineering. In particular, zinc-finger recombinases (ZFRs) are a class of chimeric proteins capable of introducing targeted modifications into mammalian cells. ZFRs recombine hybrid target sites that consist of two zinc-finger binding sites flanking a central 20-bp core sequence recognized by the recombinase catalytic domain. Unlike targeted nucleases and conventional SSR systems, ZFR specificity is the cooperative product of modular site-specific DNA recognition and sequence-dependent catalysis.

By using various selection strategies, recombinase mutants have been identified that allow for unrestricted recombination between minimal recognition sequences. Because zinc-finger domains can be assembled to recognize a wide variety of unique sequences, fusion of these catalytic domains with custom zinc-finger proteins allows design of hybrid recombinases with broad targeting capabilities. “Hyperactive” mutations which support co-factor (Fis) independent catalysis have been also identified. These mutations are important for the application of recombinases in non-natural systems, such as eukaryotic organisms. Despite their ability to specifically recognize DNA segments, some of the custom designed ZFRs target integration with very low specificity. Furthermore, the targeting integration efficiency of the currently designed ZFRs is typically very low (around 0.5%).

Thus, there remains a continued need for the development of new ZFRs capable of achieving higher targeting integration, while maintaining a high specificity for the target sequence.

### SUMMARY

The present disclosure provides hyperactive Gin Recombinase variants showing a high targeting integration efficiency. These variants are useful, for example, for the integration of an exogenous sequence or a deletion of an undesired sequence in the genome or an inversion of a sequence in the genome.

In a first aspect, the present disclosure provides a Gin recombinase catalytic domain variant comprising a Phe104Asn amino acid substitution with reference to a Gin recombinase catalytic domain comprising the amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35. In some embodiments, the Gin recombinase catalytic domain variant, further comprises a His106Tyr amino acid substitution. In some embodiments, the Gin recombinase catalytic domain variant comprises the amino acid sequence set forth in any one of SEQ ID NO: 36, SEQ ID NO: 37, SEQ ID NO: 38, SEQ ID NO: 39, SEQ ID NO: 56, or SEQ ID NO: 60. In some embodiments, the Gin recombinase catalytic domain variant further comprises an Ile94Val amino acid substitution. In some embodiments, the Gin recombinase catalytic domain variant comprises the amino acid sequence set forth in any one of SEQ ID NO: 42, SEQ ID NO: 44, SEQ ID NO: 48, SEQ ID NO: 52, SEQ ID NO: 57, or SEQ ID NO: 61.

In a second aspect, the present disclosure provides a polynucleotide encoding a Gin recombinase catalytic domain variant as disclosed herein. In some embodiments, the nucleic acid sequence encoding the Gin recombinase catalytic domain variant comprises the nucleotide sequence set forth in SEQ ID NO: 7.

In a third aspect, the present disclosure provides a zinc-finger recombinase comprising a Gin recombinase catalytic domain variant operatively linked to a zinc-finger nucleotide binding domain, wherein the Gin recombinase catalytic domain variant comprises a Phe104Asn amino acid substitution with reference to a Gin recombinase catalytic domain comprising the amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35. In some embodiments, the zinc finger recombinase comprises a Gin recombinase catalytic domain variant which further comprises a His106Tyr amino acid substitution. In some embodiments, the zinc finger recombinase comprises a Gin recombinase catalytic domain variant comprising the amino acid sequence set forth in any one of SEQ ID NO: 36, SEQ ID NO: 37, SEQ ID NO: 38, SEQ ID NO: 39, SEQ ID NO: 56, or SEQ ID NO: 60. In some embodiments, the zinc finger recombinase comprises a Gin recombinase catalytic domain variant which further comprises an Ile94Val amino acid substitution. In some embodiments, the zinc finger recombinase comprises a Gin recombinase catalytic domain variant comprising the amino acid sequence set forth in any one of SEQ ID NO: 42, SEQ ID NO: 44, SEQ ID NO: 48, SEQ ID NO: 52, SEQ ID NO: 57, or SEQ ID NO: 61.

In some embodiments, the zinc-finger recombinase protein is a multimeric protein. In some embodiments, the zinc-finger recombinase protein is a homomultimeric protein. In some embodiments, the zinc-finger recombinase protein is a heteromultimeric protein. In some embodiments, the zinc-finger recombinase protein is a dimeric protein. In some embodiments, the zinc-finger recombinase protein is a homodimeric protein. In some embodiments, the zinc-finger recombinase protein is a heterodimeric protein. In some embodiments, the zinc-finger recombinase is a tetrameric protein. In some embodiments, the tetrameric protein is a homotetrameric protein. In some embodiments, the tetrameric protein is a heterotetrameric protein.

In some embodiments, the zinc finger nucleotide binding domain comprises the sequence as set forth in SEQ ID NO: 9 or SEQ ID NO: 10.

In some embodiments, the zinc finger recombinase binds a nucleotide sequence comprising the sequence as set forth in SEQ ID NO: 15.

In some embodiments, the zinc finger nucleotide binding domain is capable of binding an endogenous locus. Optionally, the endogenous locus is selected from the group consisting of Hypoxanthine-guanine phosphoribosyltransferase (HPRT) gene, a T Cell Receptor Alpha Constant (TRAC) gene, an Adeno-Associated Virus Integration Site 1 (AAVS1) and a safe-harbor locus. Optionally, the endogenous locus is selected from the group consisting of Hypoxanthine-guanine phosphoribosyltransferase (HPRT) gene, a T Cell Receptor Alpha Constant (TRAC) gene and a safe-harbor locus.

In fourth aspect, the present disclosure provides a polynucleotide encoding the zinc-finger recombinase of the disclosure. In some embodiments, the nucleic acid sequence encoding the Gin recombinase catalytic domain variant comprises the nucleotide sequence set forth in SEQ ID NO: 7.

In a fifth aspect, the present disclosure provides a vector comprising the polynucleotide encoding the Gin recombinase catalytic domain variant of the disclosure or the zinc-finger recombinase of the disclosure. In some embodiments, the vector comprises the polynucleotide encoding the Gin recombinase catalytic domain variant of the disclosure. In some embodiments, the vector comprises the zinc-finger recombinase of the disclosure.

In a sixth aspect, the present disclosure provides a cell comprising the vector, Gin recombinase catalytic domain variant, polynucleotide encoding the Gin recombinase catalytic domain variant, zinc-finger recombinase or polynucleotide encoding the zinc-finger recombinase, of the disclosure. In some embodiments, the cell comprises the vector comprising the polynucleotide encoding the Gin recombinase catalytic domain variant of the disclosure or the zinc-finger recombinase of the disclosure. In some embodiments, the cell comprises the Gin recombinase catalytic domain variant of the disclosure. In some embodiments, the cell comprises the zinc finger recombinase of the disclosure. In some embodiments, the cell comprises the polynucleotide encoding the Gin recombinase catalytic domain variant of the disclosure. In some embodiments, the cell comprises the polynucleotide encoding the zinc-finger recombinase of the disclosure.

In some embodiments, the cell is a eukaryotic cell. In some embodiments, the cell is a mammalian cell. In some embodiments, the cell is a stem cell. In some embodiments, the cell is a human cell.

In a seventh aspect, the present disclosure provides a pharmaceutical composition comprising the vector, Gin recombinase catalytic domain variant, polynucleotide encoding the Gin recombinase catalytic domain variant, zinc-finger recombinase or polynucleotide encoding the zinc-finger recombinase, of the disclosure. In some embodiments, the pharmaceutical composition comprises the Gin recombinase catalytic domain variant of the disclosure or the zinc-finger recombinase of the disclosure. In some embodiments, the pharmaceutical composition comprises the Gin recombinase catalytic domain variant of the disclosure. In some embodiments, the pharmaceutical composition comprises the zinc-finger recombinase of the disclosure. In some embodiments, the pharmaceutical composition comprises a polynucleotide encoding the Gin recombinase catalytic domain variant of the disclosure or the zinc-finger recombinase of the disclosure. In some embodiments, the pharmaceutical composition comprises a polynucleotide encoding the Gin recombinase catalytic domain variant of the disclosure. In some embodiments, the pharmaceutical composition comprises the polynucleotide encoding the zinc-finger recombinase of the disclosure.

In an eighth aspect, the present disclosure provides a method for modifying the genome of a cell, the method comprising introducing into the cell a zinc-finger recombinase of the disclosure or a polynucleotide encoding the zinc-finger recombinase of the disclosure. In some embodiments, the method for modifying the genome of the cell comprises introducing into the cell the zinc-finger recombinase of the disclosure. In some embodiments, the method for modifying the genome of the cell comprises introducing into a cell the polynucleotide encoding a zinc-finger recombinase of the disclosure.

In an ninth aspect, the present disclosure provides a method for integrating an exogenous nucleotide sequence into a target nucleotide sequence in a genome of a cell, the method comprising introducing into a cell a zinc-finger recombinase of the disclosure or the polynucleotide encoding a zinc-finger recombinase of the disclosure. In some embodiments, the method for integrating an exogenous nucleotide sequence into a target nucleotide sequence in genome of a cell comprises introducing into the cell a zinc-finger recombinase of the disclosure. In some embodiments, the method for integrating an exogenous nucleotide sequence into a target nucleotide sequence in the genome of a cell, comprises introducing into the cell a polynucleotide encoding a zinc-finger recombinase of the disclosure. In some embodiments, the present disclosure provides a method for integrating an exogenous nucleotide sequence into a target nucleotide sequence in a gene of a cell, the method comprising introducing into the cell a zinc-finger recombinase of the disclosure or the polynucleotide encoding the zinc-finger recombinase of the disclosure. In some embodiments, the present disclosure provides a method for integrating an exogenous nucleotide sequence into a target nucleotide sequence in a gene of a cell the method comprising introducing into the cell a zinc-finger recombinase of the disclosure. In some embodiments, the present disclosure provides a method for integrating an exogenous nucleotide sequence into a target nucleotide sequence in a gene of a cell, the method comprising introducing into the cell a polynucleotide encoding the zinc-finger recombinase of the disclosure.

In a tenth aspect, the present disclosure provides a method for disrupting a target nucleotide sequence in a genome of a cell, the method comprising introducing into the cell a zinc-finger recombinase of the disclosure or a polynucleotide encoding the zinc-finger recombinase of the disclosure. In some embodiments, the method for disrupting a target nucleotide sequence in a genome of a cell, comprises introducing into the cell a zinc-finger recombinase of the disclosure. In some embodiments, the method for disrupting a target nucleotide sequence in a genome of a cell comprises introducing into the cell a polynucleotide encoding the zinc-finger recombinase of the disclosure. In some embodiments, the present disclosure provides a method for disrupting a target nucleotide sequence in a gene of a cell, the method comprising introducing into the cell a zinc-finger recombinase of the disclosure or a polynucleotide encoding the zinc-finger recombinase of the disclosure. In some embodiments, the method for disrupting a target nucleotide sequence in a gene of a cell, comprises introducing into the cell a zinc-finger recombinase of the disclosure. In some embodiments, the method for disrupting a target nucleotide sequence in a gene of a cell, comprises introducing into the cell a polynucleotide encoding the zinc-finger recombinase of the disclosure.

In an eleventh aspect, the present disclosure provides a method for excising a target nucleotide sequence in a genome of a cell, the method comprising introducing into the cell a zinc-finger recombinase of the disclosure or a polynucleotide encoding the zinc-finger recombinase of the disclosure. In some embodiments, the method for excising a target nucleotide sequence from the genome of a cell comprises introducing into the cell a zinc-finger recombinase of the disclosure. In some embodiments, the method for excising a target nucleotide sequence from the genome of a cell comprises introducing into a cell the polynucleotide encoding the zinc-finger recombinase of the disclosure. In some embodiments, the method for excising a target nucleotide sequence from the genome of a cell, comprises introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method for excising a target nucleotide sequence from the genome of a cell comprises introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35.

In some embodiments, the method for excising a target nucleotide sequence from the genome of a cell further comprises introducing into the cell a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

In a twelfth aspect, the present disclosure provides a method for excising a target nucleotide sequence in a chromosome of a cell, the method comprising introducing into the cell the zinc-finger recombinase of the disclosure or the polynucleotide encoding the zinc-finger recombinase of the disclosure.

In a thirteenth aspect, the present disclosure provides a method for treating a disorder in a subject, the method comprising modifying a target sequence in the genome of the cell by introducing into the cell a zinc-finger recombinase of the disclosure or a polynucleotide encoding the zinc-finger recombinase of the disclosure. In some embodiments, the method of treating a disorder in a subject, comprises modifying a target sequence in the genome of the cell by introducing into the cell a zinc-finger recombinase of the disclosure. In some embodiments, the method of treating a disorder in a subject, comprises modifying a target sequence in the genome of the cell by introducing into the cell a polynucleotide encoding a zinc-finger recombinase of the disclosure. In some embodiments, the method for treating a disorder in a subject comprises excising a target sequence from the genome of the cell by introducing into the cell a zinc-finger recombinase of the disclosure. In some embodiments, the method for treating a disorder in a subject comprises excising a target sequence from the genome of the cell a polynucleotide encoding a zinc-finger recombinase of the disclosure. In some embodiments, the method for treating a disorder in a subject comprises excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method for treating a disorder in a subject comprises excising a target sequence from the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method of treating a disorder, further comprises administering a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

In a fourteenth aspect, the present disclosure provides a method for correcting a disease-causing mutation in the genome of a cell, the method comprising modifying a target sequence in the genome of the cell comprising introducing into the cell the zinc-finger recombinase of the disclosure or the polynucleotide encoding the zinc-finger recombinase of the disclosure. In some embodiments, the method for correcting a disease-causing mutation in the genome of a cell comprises modifying a target sequence in the genome of the cell comprising introducing into the cell a zinc-finger recombinase of the disclosure. In some embodiments, the method for correcting a disease-causing mutation in the genome of a cell comprises modifying a target sequence in the genome of the cell comprising introducing into the cell a polynucleotide encoding the zinc-finger recombinase of the disclosure. In some embodiments, the method for correcting a disease-causing mutation in the genome of a cell comprises excising a target sequence from the genome of the cell by introducing into the cell a zinc-finger recombinase of the disclosure. In some embodiments, the method for correcting a disease-causing mutation in the genome of a cell comprises excising a target sequence from the genome of the cell by introducing into the cell a polynucleotide encoding a zinc-finger recombinase of the disclosure. In some embodiments, the method for correcting a disease-causing mutation in the genome of a cell comprises excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method for correcting a disease-causing mutation in the genome of a cell comprises excising a target sequence in the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method for correcting a disease-causing mutation in the genome of a cell further comprises a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from a group consisting of KU0060648, VX-984, W7, Chlorpromazine, Vanillin, Nu7026, Nu7441, Mirin, SCR7, AG14361, M9831 and VXc-296.

In some embodiments, the cell used in the methods disclosed herein is a eukaryotic cell. In some embodiments, the cell is a mammalian cell. In some embodiments, the cell is a stem cell. In some embodiments, the cell is a human cell.

In some embodiments, the methods disclosed herein are independent of Fis.

In some embodiments, the polynucleotide encoding the zinc-finger recombinase used in the methods disclosed herein is introduced into the cell using a plasmid, a viral vector, a mini-circle or a linear DNA form.

In some embodiments, the target nucleotide sequence used in the methods disclosed herein is an endogenous locus. Optionally, the endogenous locus is selected from the group consisting of Hypoxanthine-guanine phosphoribosyltransferase (HPRT) gene, T Cell Receptor Alpha Constant (TRAC), an Adeno-Associated Virus Integration Site 1 (AAVS1) and a safe-harbor locus. Optionally, the endogenous locus is selected from the group consisting of Hypoxanthine-guanine phosphoribosyltransferase (HPRT) gene, T Cell Receptor Alpha Constant (TRAC) and a safe-harbor locus.

In a fifteenth aspect, the present disclosure provides a zinc-finger recombinase of the disclosure or a polynucleotide encoding a zinc-finger recombinase of the disclosure for use in modifying the genome of a cell. In some embodiments, the present disclosure provides a zinc-finger recombinase of the disclosure for use in modifying the genome of a cell. In some embodiments, the present disclosure provides a polynucleotide encoding a zinc-finger recombinase of the disclosure for use in modifying the genome of a cell.

In a sixteenth aspect, the present disclosure provides a zinc-finger recombinase of the disclosure or a polynucleotide encoding a zinc-finger recombinase of the disclosure for use in integrating an exogenous nucleotide sequence into a target nucleotide sequence in genome of a cell. In some embodiments, the present disclosure provides a zinc-finger recombinase of the disclosure for use in integrating an exogenous nucleotide sequence into a target nucleotide sequence in genome of a cell. In some embodiments, the present disclosure provides a polynucleotide encoding a zinc-finger recombinase of the disclosure for use in integrating an exogenous nucleotide sequence into a target nucleotide sequence in the genome of a cell.

In a seventeenth aspect, the present disclosure provides a zinc-finger recombinase of the disclosure or a polynucleotide encoding a zinc-finger recombinase of the disclosure for use in disrupting a target nucleotide sequence in the genome of a cell. In some embodiments, the present disclosure provides a zinc-finger recombinase of the disclosure for use in disrupting a target nucleotide sequence in the genome of a cell. In some embodiments, the present disclosure provides a polynucleotide encoding a zinc-finger recombinase of the disclosure for use in disrupting a target nucleotide sequence in the genome of a cell.

In an eighteenth aspect, the present disclosure provides a zinc-finger recombinase of the disclosure or a polynucleotide encoding a zinc-finger recombinase of the disclosure for use in excising a target nucleotide sequence from the genome of a cell. In some embodiments, the present disclosure provides a zinc-finger recombinase of the disclosure for use in excising a target nucleotide sequence from the genome of a cell. In some embodiments, the present disclosure provides a polynucleotide encoding a zinc-finger recombinase of the disclosure for use in excising a target nucleotide sequence from the genome of a cell.

In some embodiments, the present disclosure provides a zinc finger recombinase for use in excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the present disclosure provides a polynucleotide encoding a zinc-finger recombinase for use in excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35.

In some embodiments, the zinc finger recombinase of the disclosure or polynucleotide encoding a zinc finger recombinase of the disclosure for use in excising a target nucleotide sequence from the genome of a cell, is used in combination with an non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

In a nineteenth aspect, the present disclosure provides a zinc-finger recombinase of the disclosure or a polynucleotide encoding a zinc-finger recombinase of the disclosure for use in treating a disorder in a subject by modifying a target sequence in the genome of the cell. In some embodiments, the present disclosure provides a zinc-finger recombinase of the disclosure for use in treating a disorder in a subject by modifying a target sequence in the genome of the cell. In some embodiments, the present disclosure provides a polynucleotide encoding a zinc-finger recombinase of the disclosure for use in treating a disorder in a subject by modifying a target sequence in the genome of the cell.

In a twentieth aspect, the present disclosure provides a zinc-finger recombinase of the disclosure or a polynucleotide encoding a zinc-finger recombinase of the disclosure for use in treating a disorder in a subject by excising a target sequence from the genome of the cell. In some embodiments, the present disclosure provides a zinc-finger recombinase of the disclosure for use in treating a disorder in a subject by excising a target sequence from the genome of the cell. In some embodiments, the present disclosure provides a polynucleotide encoding a zinc-finger recombinase of the disclosure for use in treating a disorder in a subject, by excising a target sequence from the genome of the cell.

In some embodiments, the present disclosure provides a zinc-finger recombinase for use in treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the present disclosure provides a polynucleotide encoding a zinc-finger recombinase for use in treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the zinc finger recombinase of the disclosure or polynucleotide encoding a zinc finger recombinase of the disclosure for use in treating a disorder in a subject, by excising a target sequence from the genome of a cell, is used in combination with a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

In a twenty-first aspect, the present disclosure provides a zinc-finger recombinase of the disclosure or a polynucleotide encoding a zinc-finger recombinase of the disclosure for use in correcting a disease-causing mutation in the genome of a cell by modifying a target sequence in the genome of the cell. In some embodiments, the present disclosure provides a zinc-finger recombinase of the disclosure for use in correcting a disease-causing mutation in the genome of a cell by modifying a target sequence in the genome of the cell. In some embodiments, the present disclosure provides a polynucleotide encoding a zinc-finger recombinase of the disclosure for use in correcting a disease-causing mutation in the genome of a cell by modifying a target sequence in the genome of the cell.

In a twenty-second aspect, the present disclosure provides a zinc-finger recombinase of the disclosure or a polynucleotide encoding a zinc-finger recombinase of the disclosure for use in correcting a disease-causing mutation in the genome of a cell by excising a target sequence from the genome of the cell. In some embodiments, the present disclosure provides a zinc-finger recombinase of the disclosure for use in correcting a disease-causing mutation in the genome of a cell by excising a target sequence from the genome of the cell. In some embodiments, the present disclosure provides a polynucleotide encoding a zinc-finger recombinase of the disclosure for use in correcting a disease-causing mutation in the genome of a cell by excising a target sequence from the genome of the cell.

In some embodiments, the present disclosure provides a zinc finger recombinase for use in correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the present disclosure provides a polynucleotide encoding a zinc-finger recombinase for use in correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the zinc finger recombinase of the disclosure or polynucleotide encoding a zinc finger recombinase of the disclosure for use in correcting a disease-causing mutation in the genome of a cell, by excising a target sequence from the genome of the cell, is used in combination with a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

In a twenty-third aspect, the present disclosure provides the use of a zinc-finger recombinase of the disclosure or a polynucleotide encoding a zinc-finger recombinase of the disclosure in the preparation of a medicament for modifying the genome of a cell. In some embodiments, the present disclosure provides the use of a zinc-finger recombinase of the disclosure in the preparation of a medicament for modifying the genome of a cell. In some embodiments, the present disclosure provides the use of a polynucleotide encoding a zinc-finger recombinase of the disclosure in the preparation of a medicament for modifying the genome of a cell.

In a twenty-fourth aspect, the present disclosure provides the use of a zinc-finger recombinase of the disclosure or a polynucleotide encoding a zinc-finger recombinase of the disclosure in the preparation of a medicament for integrating an exogenous nucleotide sequence into a target nucleotide sequence in genome of a cell. In some embodiments, the present disclosure provides the use of a zinc-finger recombinase of the disclosure in the preparation of a medicament for integrating an exogenous nucleotide sequence into a target nucleotide sequence in genome of a cell. In some embodiment, the present disclosure provides the use of a polynucleotide encoding a zinc-finger recombinase of the disclosure in the preparation of a medicament for integrating an exogenous nucleotide sequence into a target nucleotide sequence in the genome of a cell.

In a twenty-fifth aspect, the present disclosure provides the use of a zinc-finger recombinase of the disclosure or a polynucleotide encoding a zinc-finger recombinase of the disclosure in the preparation of a medicament for disrupting a target nucleotide sequence in the genome of a cell. In some embodiments, the present disclosure provides the use of a zinc-finger recombinase of the disclosure in the preparation of a medicament for disrupting a target nucleotide sequence in the genome of a cell. In some embodiments, the present disclosure provides the use of a polynucleotide encoding a zinc-finger recombinase of the disclosure in the preparation of a medicament for disrupting a target nucleotide sequence in the genome of a cell.

In a twenty-sixth aspect, the present disclosure provides the use of a zinc-finger recombinase of the disclosure or a polynucleotide encoding a zinc-finger recombinase of the disclosure in the preparation of a medicament for excising a target nucleotide sequence from the genome of a cell. In some embodiments, the present disclosure provides the use of a zinc-finger recombinase of the disclosure in the preparation of a medicament for excising a target nucleotide sequence from the genome of a cell. In some embodiments, the present disclosure provides the use of a polynucleotide encoding a zinc-finger recombinase of the disclosure in the preparation of a medicament for excising a target nucleotide sequence from the genome of a cell.

In some embodiments, the present disclosure provides the use of a zinc finger recombinase in the preparation of a medicament for excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the present disclosure provides the use of a polynucleotide encoding a zinc-finger recombinase in the preparation of a medicament for excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35.

In some embodiments, the use of the zinc finger recombinase or polynucleotide encoding the zinc finger recombinase of the disclosure, in the preparation of a medicament for excising a target nucleotide sequence from the genome of a cell further comprises the use of a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

In a twenty-seventh aspect, the present disclosure provides the use of a zinc-finger recombinase of the disclosure or a polynucleotide encoding a zinc-finger recombinase of the disclosure in the preparation of a medicament for treating a disorder in a subject by modifying a target sequence in the genome of the cell. In some embodiments, the present disclosure provides the use of a zinc-finger recombinase of the disclosure in the preparation of a medicament for treating a disorder in a subject by modifying a target sequence in the genome of the cell. In some embodiments, the present disclosure provides the use of a polynucleotide encoding a zinc-finger recombinase of the disclosure in the preparation of a medicament for treating a disorder in a subject by modifying a target sequence in the genome of the cell.

In a twenty eighth aspect, the present disclosure provides the use of a zinc-finger recombinase of the disclosure or a polynucleotide encoding a zinc-finger recombinase of the disclosure in the preparation of a medicament for treating a disorder in a subject by excising a target sequence from the genome of the cell. In some embodiments, the present disclosure provides the use of a zinc-finger recombinase of the disclosure in the preparation of a medicament for treating a disorder in a subject by excising a target sequence from the genome of the cell. In some embodiments, the present disclosure provides the use of a polynucleotide encoding a zinc-finger recombinase of the disclosure in the preparation of a medicament for treating a disorder in a subject, by excising a target sequence from the genome of the cell.

In some embodiments, the present disclosure provides the use of a of a zinc-finger recombinase or a polynucleotide encoding a zinc-finger recombinase of the disclosure in the preparation of a medicament for treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the present disclosure provides the use of a polynucleotide encoding a zinc-finger recombinase in the preparation of a medicament for treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the use of a zinc finger recombinase of the disclosure or polynucleotide encoding a zinc finger recombinase of the disclosure for the preparation of a medicament for treating a disorder in a subject, by excising a target sequence from the genome of a cell, is used in combination with a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

In a twenty-ninth aspect, the present disclosure provides the use of a zinc-finger recombinase of the disclosure or a polynucleotide encoding a zinc-finger recombinase of the disclosure in the preparation of a medicament for correcting a disease-causing mutation in the genome of a cell by modifying a target sequence in the genome of the cell. In some embodiments, the present disclosure provides the use of a zinc-finger recombinase of the disclosure in the preparation of a medicament for correcting a disease-causing mutation in the genome of a cell by modifying a target sequence in the genome of the cell. In some embodiments, the present disclosure provides the use of a polynucleotide encoding a zinc-finger of the disclosure in the preparation of a medicament for correcting a disease-causing mutation in the genome of a cell by modifying a target sequence in the genome of the cell.

In a thirtieth aspect, the present disclosure provides the use of a zinc-finger recombinase of the disclosure or a polynucleotide encoding a zinc-finger recombinase of the disclosure in the preparation of a medicament for correcting a disease-causing mutation in the genome of a cell by excising a target sequence from the genome of the cell. In some embodiments, the present disclosure provides the use of a zinc-finger recombinase of the disclosure in the preparation of a medicament for correcting a disease-causing mutation in the genome of a cell by excising a target sequence from the genome of the cell. In some embodiments, the present disclosure provides the use of a polynucleotide encoding a zinc-finger recombinase of the disclosure in the preparation of a medicament for correcting a disease-causing mutation in the genome of a cell.

In some embodiments, the present disclosure provides the use of a zinc finger recombinase in the preparation of a medicament for correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the present disclosure provides the use of a polynucleotide encoding a zinc-finger recombinase in the preparation of a medicament for correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the use of zinc finger recombinase of the disclosure or polynucleotide encoding a zinc finger recombinase of the disclosure for the preparation of a medicament for correcting a disease-causing mutation in the genome of a cell by excising a target sequence from the genome of the cell, is in combination with a non-homologous end joining (NHEJ) inhibitor. In some embodiments, the NHEJ inhibitor is selected from the group consisting of a small molecule inhibitor, a zinc-finger protein transcription factor (ZFP-TF), and a peptide inhibitor. In some embodiments, the small molecule inhibitor is selected from the group consisting of KU0060648, VX-984, W7, Chlorpromazine, Vanillin, Nu7026, Nu7441, Mirin, SCR7, AG14361, M9831 and VXc-296.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing the results of a Standard Next Generation sequencing (NGS)-based Target Integration assay into HPRT gene in K562 cells, where the target integration rate (% TI) is depicted on the left and the % Indel (Insertion-deletion mutations) is depicted on the right. The graph compares the integration catalyzed by the zinc finger recombinase F104N variant compared with the H106Y recombinase variant and no recombinase (GFP).

FIG. 2 shows the results of a BL1-A (blue laser fluorescence channel 1) flow cytometry assay where K562 cells were transfected with a donor vector comprising a GFP expression cassette and the F104N ZFR variant targeting HPRT gene (Panel C), and compared with cells transfected with only a donor vector comprising a GFP expression cassette (Panel B) and untransfected cells (Panel A).

FIG. 3 shows a schematic of the DNA strand exchange mechanism of serine recombinases and of the Gin recombinase of the disclosure, the first step is the dimer formation at target and donor sites (Panel A); each of the dimers specifically bind the target and the donor sites and a tetramer is formed between the target dimer and the donor dimer, which catalyzes the cleavage (Panel B); subunit-rotation around the tetramer interface permits the strand exchange while each recombinase subunit stays covalently linked to the DNA (Panel C); and religation completes the stable integration of the donor sequence into the target sequence (targeted integration) (Panel D).

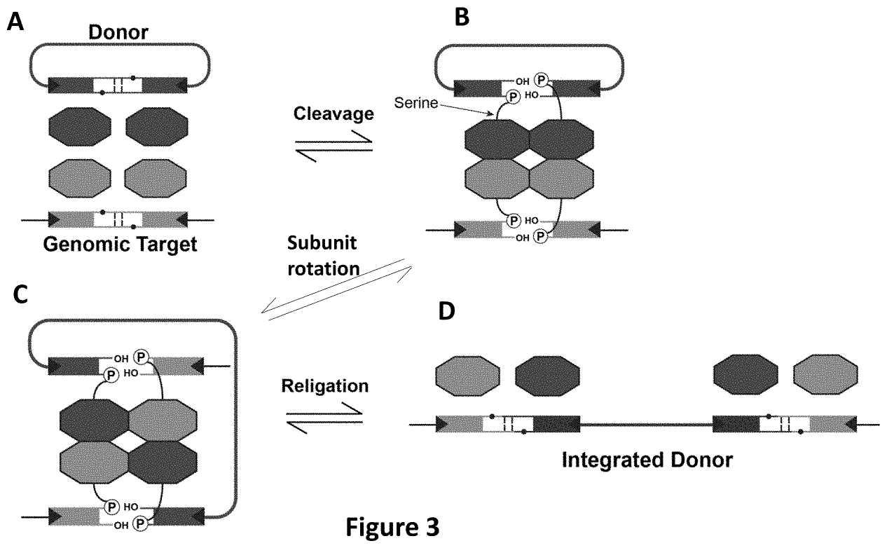

FIG. 4 shows that ZFR TI mechanism is distinct from ZFN/NHEJ-mediated TI. Panel A is a schematic of nuclease- and recombinase-mediated TI in the presence of a NHEJ inhibitor; % Indel, % perfect TI, and % total TI were measured following co-transfection of K562 cells with HPRT ZFN control or HPRT ZFR constructs (F104N:I94V) and donor plasmids, in the presence or absence of an NHEJ inhibitor (KU00060648) (Panel B).

FIG. 5 shows Indel-free ZFR mediated excision with C-NHEJ inhibitor.

Panel A shows a schematic of ZFR-mediated excision. Panel B shows results of % Indels, % total excision and % perfect excision in a K562 reporter cell line with two HPRT ZFR binding sites transfected with H106Y ZFR, F104N ZFR, or ZFN constructs in the presence or absence of a NHEJ inhibitor (KU0060648).

FIG. 6 is a schematic of zinc finger recombinase (ZFR) binding to a target nucleotide sequence and forming a ZFR dimer. The ZFP and Gin catalytic domain of each ZFR binds to their respective ZFP binding domain and Gin binding site in the ZFR target nucleotide sequence.

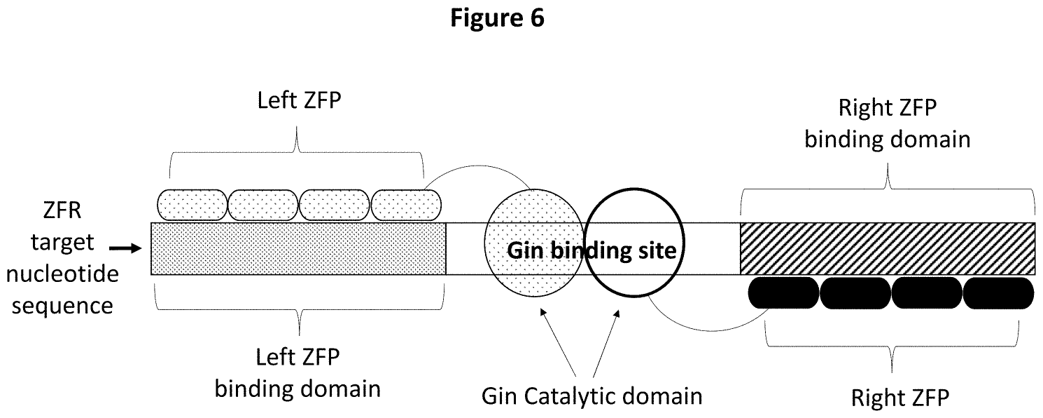

### DETAILED DESCRIPTION

The present disclosure provides serine recombinase variants having improved targeting integration efficiency, compositions comprising said recombinase variants and methods of editing the genome by either integrating an exogenous sequence, excising an endogenous sequence or by disrupting an undesired sequence.

By using various selection strategies, hyperactivated ZFR mutants have been identified. The custom designed ZFRs target integration of exogenous sequences with high precision and a targeting integration efficiency closer to 20%.

The present disclosure describes zinc-finger recombinase variant proteins which are capable of integrating an exogenous nucleotide sequence or excising an endogenous target nucleotide sequence with high precision and targeting integration efficiency; a cell and a pharmaceutical composition comprising said recombinase; methods for modifying the genome of a cell; methods for integration of an exogenous nucleotide sequence and methods for deleting a target sequence using said recombinase variant.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the invention, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including”, as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “binding”, as used herein, refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}. “Non-specific binding” refers to, non-covalent interactions that occur between any molecule of interest (e.g. an engineered nuclease) and a macromolecule (e.g. DNA) that are not dependent on-target sequence.

The term “chromosome”, as used herein, refers to a chromatin complex comprising all or a portion of the genome of a cell. The genome of a cell is often characterized by its karyotype, which is the collection of all the chromosomes that comprise the genome of the cell. The genome of a cell can comprise one or more chromosomes.

The term “cleavage”, as used herein, refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “DNA binding molecule”, as used herein, refers to a molecule that can bind to DNA. Such DNA binding molecule can be a polypeptide, a domain of a protein, a domain within a larger protein or a polynucleotide. In some embodiments, the polynucleotide is DNA, while in other embodiments, the polynucleotide is RNA. In some embodiments, the DNA binding molecule is a protein domain of a nuclease (e.g. the zinc finger domain).

A “DNA binding protein” or “binding domain”, as used herein, refers to a protein, or a domain within a larger protein, that binds DNA in a sequence-specific manner, for example through one or more zinc fingers or through interaction with one or more Repeat Variable Diresidue (RVDs) in a zinc finger protein or TALE, respectively.

A “zinc-finger DNA binding protein” or “zinc-finger nucleotide binding domain”, as used herein, refers to a protein, or a domain within a larger protein, that binds DNA in a sequence-specific manner through one or more zinc fingers, which are regions of amino acid sequence within the binding domain whose structure is stabilized through coordination of one or more zinc ions. The term zinc finger DNA binding protein is often abbreviated as zinc finger protein or ZFP.

An “exogenous” sequence is a sequence that is not normally present in the genome of a specific cell, but can be introduced into a cell by one or more delivery methods. An exogenous nucleic acid can comprise a therapeutic gene, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous nucleic acid can also be the same type of molecule as an endogenous molecule but derived from a different species than the cell is derived from. For example, a human nucleic acid sequence may be introduced into a cell line originally derived from a mouse or hamster.

As used herein, the term “product of an exogenous nucleic acid” includes both polynucleotide and polypeptide products, for example, transcription products (polynucleotides such as RNA) and translation products (polypeptides).

An “endogenous” molecule or sequence is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

“Eukaryotic” cells include, but are not limited to, fungal cells (such as yeast), plant cells, animal cells, mammalian cells and human cells (e.g., T-cells), including stem cells (pluripotent and multipotent).

The terms “excision,” “targeted excision,” “deletion” and “targeted deletion” are used interchangeably herein and refer to the removal of a DNA or nucleotide sequence of interest for its endogenous locus. The nucleotide sequence of interest may be a duplicate nucleotide sequence (also referred to as a duplication event) in the genome. The targeted nucleotide sequence to be excised can range from less than 100 bases to megabases).

A “fusion” molecule or any variation thereof is a molecule in which two or more subunit molecules are linked, preferably covalently. The subunit molecules can be the same chemical type of molecule or can be different chemical types of molecules. Examples of fusion molecules include, but are not limited to, fusion proteins (for example, a fusion between a zinc-finger DNA binding domain and a recombinase), and fusion nucleic acids (for example, a nucleic acid encoding the fusion protein).

A “gene,” as used herein, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression”, as used herein, refers to the conversion of the information contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

“Flanking”, as used herein, refers to a nucleotide sequence that is located directly 5′ (i.e., upstream) or 3′ (i.e., downstream) of a target nucleotide sequence or nucleotide sequence of interest. For example, two ZFR binding sites can flank the 5′ and 3′ ends of a target nucleotide sequence to be excised. In the context of amino acid sequences, “Flanking” refers to an amino acid sequence that is located N-terminal (upstream) or C-terminal (downstream) of a target amino sequence or amino acid sequence of interest.

“Recombinases” are a family of enzymes that catalyze site-specific recombination events within DNA sequences (see, e.g., Esposito, D., and Scocca, J. J., Nucleic Acids Research 25, 3605-3614 (1997); Nunes-Duby, S. E., et al., Nucleic Acids Research 26, 391-406 (1998); Stark, W. M., et al., Trends in Genetics 8, 432-439 (1992)).

“Gin recombinase” or “Gin recombinase protein”, as used herein, refers to an enzyme (serine recombinase) found in Escherichia bacteriophage Mu. The enzyme is capable of performing inversion of a viral segment (G-segment) that encodes two alternate pairs of tail fiber proteins thereby modifying the host specificity of the virus. In bacteria, the enzyme binds as a dimer to the viral gix sites which are 34-bp palindromic sequences that flank the invertible G-segment and catalyzes site-specific recombination in the presence of the host factor Fis. Gin dimers bound to each of the gix sites and host factor Fis bound to the enhancer come together to form the synaptic complex and each Gin monomer introduces a nick and becomes covalently attached to the 5′-phosphate of the DNA, resulting in double-stranded staggered breaks at both recombination sites. A 180 degrees rotation around the tetramer interface followed by religation of the DNA leads to the inversion of the G-segment (G+ or G− orientation). Plasterk R. H. et al., Proc. Natl. Acad. Sci. U.S.A. 81:2689-2692 (1984).

The term “Gin recombinase catalytic domain”, as used herein, refers to the catalytic domain of the Gin recombinase. The Gin catalytic domain recognizes a 20-bp core sequence flanked by two zinc-finger protein binding-sites. Smith M C et al., Mol Microbiol; 44:299-307 (2002). The Gin recombinase catalytic domain mediates the excision and integration of a desired nucleotide sequence by catalyzing inversion between two specific recombination sites on the same DNA molecule. The term “hyperactive” or “hyperactivated” mutant recombinase is used to indicate that the mutant is capable of co-factor (Fis) independent recombinase activity. The mutated residues giving result to hyperactivated variants typically lie near the active site serine and may enhance catalysis by optimally positioning DNA for cleavage and strand cleavage. Examples of hyperactive substitutions or mutations of Gin recombinase catalytic domain include, but are not limited to, D12G, N145, K50E, M70V, I94V and H106Y or combinations thereof. Several Gin recombinase variants have been developed, including alpha (α), beta (β), gamma (γ), delta (δ), epsilon (ε) and zeta (ζ), having increased targeting range due to relaxed or altered core sequence requirements. A Gin recombinase catalytic domain, as used here, can refer to the catalytic domain of any of the α, β, γ, δ, ε or variants. In some embodiments, the Gin recombinase catalytic domain is a γ Gin recombinase catalytic domain. The terms “Gin recombinase catalytic domain variant” and “Gin recombinase variant” are used interchangeably herein.

The term “heterologous” means derived from a genotypically distinct entity from that of the rest of the entity to which it is being compared. For example, a polynucleotide introduced by genetic engineering techniques into a plasmid or vector derived from a different species is a heterologous polynucleotide.

The term “% Indel”, as used herein, refers to the percentage of insertions or deletions of several nucleotides in the target sequence of the genome.

“Modulation” (or variants thereof) of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, TALE or CRISPR/Cas system as described herein. Thus, gene inactivation may be partial or complete.

The terms “operative linkage” and “operatively linked” (or “operably linked”) or variations thereof, as used herein, are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a Gin recombinase catalytic domain is operatively linked to a zinc-finger nucleotide binding domain. The Gin recombinase catalytic domain is operatively linked in cis with the zinc-finger nucleotide binding domain, but need not be directly adjacent to it. For example, a linker sequence can be located between both sequences.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “safe-harbor locus or site”, as used herein, is a genomic locus where genes or other genetic elements can be safely inserted and expressed, because they are known to be tolerant to genetic modification without any undesired effects.

The term “sequence” refers to a nucleotide sequence of any length, which can be DNA or RNA; can be linear, circular or branched and can be either single-stranded or double-stranded. The term “sequence” also refers to an amino acid sequence of any length. The term “transgene” refers to a nucleotide sequence that is inserted into a genome. A transgene can be of any length, for example between 2 and 100,000,000 nucleotides in length (or any integer value therebetween or thereabove), between about 100 and 100,000 nucleotides in length (or any integer therebetween), between about 2000 and 20,000 nucleotides in length (or any value therebetween) or between about 5 and 15 kb (or any value therebetween).

The term “specificity” (or variations thereof), as used herein, refers to the recombinase being able to bind the target sequence in a specific location with precision. The terms “specificity” and “precision” are used interchangeably.

The terms “subject” and “patient” are used interchangeably and refer to mammals including, but not limited to, human patients and non-human primates, as well as experimental animals such as rabbits, dogs, cats, rats, mice, and other animals. Accordingly, the term “subject” or “patient” as used herein means any mammalian patient or subject to which the expression cassettes of the invention can be administered. Subjects of the present invention include those with a disorder.

The term “target nucleotide sequence”, as used herein, refers to a nucleotide sequence located in the genome of a cell which is specifically recognized by a zinc finger nucleotide binding domain of the zinc finger recombinase protein of the disclosure.

The terms “treating” and “treatment” or variations thereof as used herein refer to reduction in severity and/or frequency of symptoms, elimination of symptoms and/or underlying cause, prevention of the occurrence of symptoms and/or their underlying cause, and improvement or remediation of damage. Cancer, monogenic diseases and graft versus host disease are non-limiting examples of conditions that may be treated using the compositions and methods described herein.

A polynucleotide “vector” or “construct” is capable of transferring gene sequences to target cells. Typically, “vector construct,” “expression vector,” “expression construct,” “expression cassette,” and “gene transfer vector,” mean any nucleic acid construct capable of directing the expression of a gene of interest and which can transfer gene sequences to target cells. Thus, the term includes cloning, and expression vehicles, as well as integrating vectors.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “zinc-finger nuclease protein” or “zinc-finger nuclease”, as used herein, refers to a protein comprising a zinc-finger DNA binding domain (ZFP) directly or indirectly linked to a DNA cleavage domain (e.g., a Fok I DNA cleavage domain). The term zinc-finger nuclease protein is abbreviated as zinc finger nuclease or ZFN. The cleavage domain may be connected directly to the ZFP. Alternatively, the cleavage domain is connected to the ZFP by way of an intervening sequence (e.g., a linker). The linker region is a sequence which comprises about 1-150 amino acids. Alternatively, the linker region is a sequence which comprises about 6-50 nucleotides. The term includes one ZFN as well as a pair of ZFNs (the members of the pair are referred to as “left and right” or “first and second” or “pair”) that dimerize to cleave the target gene. A pair of ZFNs can be referred to as “left and right”, “first and second” or “pair” and can dimerize to cleave a target gene.

A “zinc-finger recombinase protein” or “zinc-finger recombinase”, as used herein, is a hybrid recombinase protein comprising the catalytic domain of a recombinase protein (e.g., a serine recombinase, including e.g., a Gin recombinase) operatively linked to a zinc-finger nucleotide binding domain. The term zinc-finger recombinase protein is often abbreviated as zinc finger recombinase or ZFR. The zinc-finger recombinase protein may be multimeric (e.g., homomultimer or heteromultimer). In some embodiments, the zinc-finger recombinase protein is a dimer. In some embodiments, the zinc-finger recombinase protein is a homodimer. In some embodiments, the homodimer comprises two gamma Gin recombinases. In some embodiments, the zinc-finger recombinase protein is a heterodimer. In some embodiments, the heterodimer comprises a monomer comprising gamma Gin recombinase and another monomer comprising a Gin recombinase variant selected from the group of alpha (a), beta (13), delta (6), epsilon (c) and zeta Q. In some embodiments, the zinc-finger recombinase protein is a tetramer. A ZFR target site generally includes of two zinc-finger protein binding-sites flanking a 20-bp core sequence recognized by the serine recombinase catalytic domain.

Gin Recombinases

In one aspect, the subject matter disclosed herein relates to a Gin recombinase catalytic domain variant comprising a Phe104Asn amino acid substitution with reference to a Gin recombinase catalytic domain comprising the amino acid sequence set forth in any one of sequences SEQ ID NO: 1, SEQ ID NO: 31, SEQ ID NO: 32, SEQ ID NO: 33, SEQ ID NO: 34 or SEQ ID NO: 35.

In some embodiments, the Gin recombinase catalytic domain variant comprises a substitution of Phenylalanine 104 by Asparagine, with reference to a Gin recombinase catalytic domain comprising the amino acid as set forth in SEQ ID NO: 1. In some embodiments, the Gin recombinase catalytic domain variant comprises a substitution of Phenylalanine 104 by Asparagine, with reference to a Gin recombinase catalytic domain comprising the amino acid as set forth in SEQ ID NO: 31. In some embodiments, the Gin recombinase catalytic domain variant comprises a substitution of Phenylalanine 104 by Asparagine, with reference to a Gin recombinase catalytic domain comprising the amino acid as set forth in SEQ ID NO: 32. In some embodiments, the Gin recombinase catalytic domain variant comprises a substitution of Phenylalanine 104 by Asparagine, with reference to a Gin recombinase catalytic domain comprising the amino acid as set forth in SEQ ID NO: 33. In some embodiments, the Gin recombinase catalytic domain variant comprises a substitution of Phenylalanine 104 by Asparagine, with reference to a Gin recombinase catalytic domain comprising the amino acid as set forth in SEQ ID NO: 34. In some embodiments, the Gin recombinase catalytic domain variant comprises a substitution of Phenylalanine 104 by Asparagine, with reference to a Gin recombinase catalytic domain comprising the amino acid as set forth in SEQ ID NO: 35.

In some embodiments, the Gin recombinase catalytic domain variant only comprises a Phe104Asn amino acid substitution with reference to a Gin recombinase catalytic domain comprising the amino acid sequence set forth in any one of sequences SEQ ID NO: 1, SEQ ID NO: 31, SEQ ID NO: 32, SEQ ID NO: 33, SEQ ID NO: 34 or SEQ ID NO: 35. In some embodiments, the Gin recombinase catalytic domain variant only comprises a Phe104Asn amino acid substitution with reference to a Gin recombinase catalytic domain comprising the amino acid sequence set forth in any one of sequences SEQ ID NO: 1, SEQ ID NO: 31, SEQ ID NO: 32, SEQ ID NO: 33, SEQ ID NO: 34 or SEQ ID NO: 35 and no other hyperactive mutations (e.g., D12G, N145, K50E, M70V, I94V and H106Y). In some embodiments, the Gin recombinase catalytic domain variant comprises further modifications (e.g, substitutions, insertions, deletions). Optionally, the Gin recombinase catalytic domain variant comprises a His106Tyr amino acid substitution with reference to a Gin recombinase catalytic domain comprising the amino acid sequence as set forth in SEQ ID NO: 1, SEQ ID NO: 31, SEQ ID NO: 32, SEQ ID NO: 33, SEQ ID NO: 34 or SEQ ID NO: 35. Optionally, the Gin recombinase catalytic domain variant comprises a Phe104Asn amino acid substitution and a His106Tyr amino acid substitution with reference to a Gin recombinase catalytic domain comprising the amino acid as set forth in SEQ ID NO: 1, SEQ ID NO: 31, SEQ ID NO: 32, SEQ ID NO: 33, SEQ ID NO: 34 or SEQ ID NO: 35. Optionally, the Gin recombinase catalytic domain variant comprises a Ile94Val amino acid substitution with reference to a Gin recombinase catalytic domain comprising the amino acid as set forth in SEQ ID NO: 1, SEQ ID NO: 31, SEQ ID NO: 32, SEQ ID NO: 33, SEQ ID NO: 34 or SEQ ID NO: 35. Optionally, the Gin recombinase catalytic domain variant comprises a Phe104Asn amino acid substitution and a Ile94Val amino acid substitution with reference to a Gin recombinase catalytic domain comprising the amino acid as set forth in SEQ ID NO: 1, SEQ ID NO: 31, SEQ ID NO: 32, SEQ ID NO: 33, SEQ ID NO: 34 or SEQ ID NO: 35.

In some embodiments, the Gin recombinase catalytic domain variant comprises the amino acid sequence set forth below (SEQ ID NO: 31):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERTMAGLAAARNKGRIGGRPPKSG

In some embodiments, the Gin recombinase catalytic domain variant comprises the amino acid sequence set forth below (SEQ ID NO: 32):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERTMAGIAAARNKGRRFGRPPKSG

In some embodiments, the Gin recombinase catalytic domain variant comprises the amino acid sequence set forth below (SEQ ID NO: 33):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERVMAGLAAARNKGRRFGRPPKSG

In some embodiments, the Gin recombinase catalytic domain variant comprises the amino acid sequence set forth below (SEQ ID NO: 34):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRFFFHVMGALAEMERLSILERPMAGHAAARNKGRRFGRPPKSG

In some embodiments, the Gin recombinase catalytic domain variant comprises the amino acid sequence set forth below (SEQ ID NO:35):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERTSAGRAAAINKGRIMGRPRKSG

In some embodiments, the Gin recombinase catalytic domain variant comprises the amino acid sequence set forth below (SEQ ID NO: 1):

MLIGYVRVST NDQNTDLQRN ALVCAGCEQI FEDKLSGTRT
DRPGLKRALK RLQKGDTLVV WKLDRLGRSM KHLISLVGEL
RERGINFRSL TDSIDTSSPM GRFFFHVMGA LAEMERELIL
ERVMAGIAAA RNKGRRWGRP P

In some embodiments, the Gin recombinase catalytic domain variant comprises the sequence set forth in SEQ ID NO: 3, which comprises Phe104Asn substitution with respect to the Gin recombinase catalytic domain (SEQ ID NO: 1). In some embodiments, the Gin recombinase catalytic domain variant comprises an amino acid sequence having at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference SEQ ID NO: 3, as determined by sequence alignment programs known by skilled artisans. In some embodiments, the Gin recombinase catalytic domain variant comprises an amino acid sequence set forth in SEQ ID NO: 3, which comprises Phe104Asn substitution with respect to the Gin recombinase catalytic domain (SEQ ID NO: 1). In some embodiments, the Gin recombinase catalytic domain variant comprises an amino acid sequence set forth in SEQ ID NO: 3, but does not comprise other hyperactive mutations, such as D12G or H106Y.

In some embodiments, the polynucleotide sequence encoding the Gin recombinase catalytic domain variant comprises the sequence set forth in SEQ ID NO: 6 below:

atg ctg atc ggc tat gtg cgg gtg tcc acc aac gac
cag aac acc gac ctg cag aga aac gcc ctt gtg tgt
gcc gga tgc gag cag atc ttc gag gat aag ctg agc
ggc acc aga acc gac aga ccc gga ctg aag aga gcc
ctg aag aga ctg cag aaa ggc gac acc ctg gtc gtg
tgg aag ctg gat aga ctg ggc cgc agc atg aag cac
ctg atc agc ctt gtg ggc gag ctg aga gag cgg ggc
atc aac ttt aga agc ctg acc gac agc atc gac aca
agc agc cct atg ggc aga ttc ttc ttc tac gtg atg
ggc gcc ctg gcc gag atg gaa aga gag ctg atc ctg
gag aga gtc atg gcc gga atc gcc gct gcc aga aac
aag gga aga aga tgg ggc cgg cca cct aag tct ggc
aca ggc gag agg ccc ttc cag tgt cga atc tgc atg

In some embodiments, the polynucleotide sequence encoding the Gin recombinase catalytic domain variant comprises the sequence set forth in SEQ ID NO: 7 below, which encodes the Gin recombinase catalytic domain which comprises Phe104Asn substitution with respect to the Gin recombinase catalytic domain (SEQ ID NO: 6):

atg ctg atc ggc tat gtg cgg gtg tcc acc aac gac
cag aac acc gac ctg cag aga aac gcc ctt gtg tgt
gcc gga tgc gag cag atc ttc gag gat aag ctg agc
ggc acc aga acc gac aga ccc gga ctg aag aga gcc
ctg aag aga ctg cag aaa ggc gac acc ctg gtc gtg
tgg aag ctg gat aga ctg ggc cgc agc atg aag cac
ctg atc agc ctt gtg ggc gag ctg aga gag cgg ggc
atc aac ttt aga agc ctg acc gac agc atc gac aca
agc agc cct atg ggc aga ttc aac ttc cat gtg atg
ggc gcc ctg gcc gag atg aga tgg ggc cgg cca cct

In some embodiments, the Gin recombinase nucleotide sequence of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference SEQ ID NO: 7, as determined by sequence alignment programs known by skilled artisans.

Also provided herein are vectors comprising polynucleotide sequences encoding the Gin recombinase catalytic domain variant as described herein. Suitable vectors include, but are not limited to, a plasmid, a viral vector, a mini-circle and a linear DNA form. Host cells containing said polynucleotide sequences or vectors are also provided. Any of the foregoing Gin recombinase catalytic domain variants, polynucleotides encoding the zinc-finger recombinase protein, vectors or cells may be used in the methods disclosed herein.

Another aspect of the present disclosure relates to a pharmaceutical composition comprising a Gin recombinase catalytic domain variant as disclosed herein; and a pharmaceutically acceptable carrier.

Another aspect of the present disclosure relates to a pharmaceutical composition comprising a polynucleotide encoding the Gin recombinase variant as disclosed herein; and a pharmaceutically acceptable carrier.

Zinc-Finger Recombinases

In one aspect, the subject matter disclosed herein relates to the identification of a zinc-finger recombinase (ZFR) variant which maintains a high specificity for a target sequence and a high target integration rate in the target sequence. Thus, described herein are zinc-finger recombinase proteins, comprising a Gin recombinase catalytic domain variant operatively linked to a zinc finger nucleotide binding domain, wherein the Gin recombinase catalytic domain variant comprises a Phe104Asn amino acid substitution with reference to a Gin recombinase catalytic domain amino acid sequence set forth in any one of sequences SEQ ID NO: 1, SEQ ID NO: 31, SEQ ID NO: 32, SEQ ID NO: 33, SEQ ID NO: 34 or SEQ ID NO: 35.

The zinc-finger recombinase variant of the disclosure binds a target nucleotide sequence as a dimer, interacting with the target sequence through the catalytic domain, wherein the DNA-binding domains of each of the two monomers interact with the target nucleotide sequences, whereas a second zinc-finger recombinase variant dimer interacts with the donor nucleotide sequence. The dimers may be homodimers or heterodimers (e.g., comprising two gamma-gamma Gin recombinase variants or one gamma Gin recombinase variant and one delta or alpha or beta or epsilon or zeta Gin recombinase variant).

In some embodiments, the zinc-finger recombinase variant comprises a Gin serine recombinase catalytic domain variant and a heterologous DNA binding domain (a zinc-finger nucleotide binding domain). In some embodiments, the Gin recombinase catalytic domain variant included in the ZFR comprises a substitution of Phenylalanine 104 by Asparagine, with reference to a Gin recombinase catalytic domain comprising the amino acid as set forth in SEQ ID NO: 1. In some embodiments, the Gin recombinase catalytic domain variant included in the ZFR comprises an amino acid sequence as set forth in SEQ ID NO: 3. In some embodiments, the Gin recombinase catalytic domain variant included in the ZFR comprises further modifications (e.g., substitutions, insertions, deletions). Optionally, the Gin recombinase catalytic domain variant included in the ZFR comprises a His106Tyr amino acid substitution with reference to a Gin recombinase catalytic domain comprising the amino acid as set forth in SEQ ID NO: 1. Optionally, the Gin recombinase catalytic domain variant included in the ZFR comprises a Phe104Asn and His106Tyr amino acid substitution with reference to a Gin recombinase catalytic domain comprising the amino acid as set forth in SEQ ID NO: 1. Optionally, the Gin recombinase catalytic domain variant included in the ZFR comprises a His106Tyr amino acid substitution with reference to a Gin recombinase catalytic domain comprising the amino acid as set forth in SEQ ID NO: 3.

In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence set forth in SEQ ID NO: 31. In some embodiments, the Gin recombinase catalytic domain variant included in the ZFR is a Gin recombinase catalytic domain comprising a His106Tyr amino acid substitution with reference to a Gin recombinase catalytic domain comprising the amino acid as set forth in SEQ ID NO: 31. In some embodiments, the Gin recombinase catalytic domain variant included in the ZFR is a Gin recombinase catalytic domain comprising a Phe104Asn and His106Tyr amino acid substitution with reference to a Gin recombinase catalytic domain comprising the amino acid as set forth in SEQ ID NO: 31. In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence set forth in SEQ ID NO: 32. In some embodiments, the Gin recombinase catalytic domain variant included in the ZFR is a Gin recombinase catalytic domain comprising a His106Tyr amino acid substitution with reference to a Gin recombinase catalytic domain comprising the amino acid as set forth in SEQ ID NO: 32. In some embodiments, the Gin recombinase catalytic domain variant included in the ZFR is a Gin recombinase catalytic domain comprising a Phe104Asn and His106Tyr amino acid substitution with reference to a Gin recombinase catalytic domain comprising the amino acid as set forth in SEQ ID NO: 32. In some embodiments, the Gin recombinase catalytic variant domain included in the ZFR is a Gin recombinase catalytic domain comprising a Phe104Asn amino acid substitution with reference to a Gin recombinase catalytic domain amino acid comprising the amino acid sequence set forth in SEQ ID NO: 33. In some embodiments, the Gin recombinase catalytic domain variant included in the ZFR is a Gin recombinase catalytic domain comprising a His106Tyr amino acid substitution with reference to a Gin recombinase catalytic domain comprising the amino acid as set forth in SEQ ID NO: 33. In some embodiments, the Gin recombinase catalytic domain variant included in the ZFR is a Gin recombinase catalytic domain comprising a Phe104Asn and His106Tyr amino acid substitution with reference to a Gin recombinase catalytic domain comprising the amino acid as set forth in SEQ ID NO: 33. In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence as set forth in SEQ ID NO: 34. In some embodiments, the Gin recombinase catalytic domain variant included in the ZFR is a Gin recombinase catalytic domain comprising a His106Tyr amino acid substitution with reference to a Gin recombinase catalytic domain comprising the amino acid as set forth in SEQ ID NO: 34. In some embodiments, the Gin recombinase catalytic domain variant included in the ZFR is a Gin recombinase catalytic domain comprising a Phe104Asn and His106Tyr amino acid substitution with reference to a Gin recombinase catalytic domain comprising the amino acid as set forth in SEQ ID NO: 34. In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence as set forth in SEQ ID NO: 35. In some embodiments, the Gin recombinase catalytic domain variant included in the ZFR is a Gin recombinase catalytic domain comprising a His106Tyr amino acid substitution with reference to a Gin recombinase catalytic domain comprising the amino acid as set forth in SEQ ID NO: 35. In some embodiments, the Gin recombinase catalytic domain variant included in the ZFR is a Gin recombinase catalytic domain comprising a Phe104Asn and His106Tyr amino acid substitution with reference to a Gin recombinase catalytic domain comprising the amino acid as set forth in SEQ ID NO: 35.

In some embodiments, the zinc-finger recombinase variant comprises a Gin serine recombinase catalytic domain variant and a heterologous DNA binding domain (a zinc-finger nucleotide binding domain). In some embodiments, the Gin recombinase catalytic domain variant included in the ZFR comprises a substitution of Phenylalanine 104 by Asparagine, with reference to a Gin recombinase catalytic domain comprising the amino acid as set forth in SEQ ID NO: 1. In some embodiments, the Gin recombinase catalytic domain variant included in the ZFR comprises an amino acid sequence as set forth in SEQ ID NO: 3. In some embodiments, the Gin recombinase catalytic domain variant included in the ZFR comprises further modifications (e.g., substitutions, insertions, deletions). Optionally, the Gin recombinase catalytic domain variant included in the ZFR comprises a Ile94Val amino acid substitution with reference to a Gin recombinase catalytic domain comprising the amino acid as set forth in SEQ ID NO: 1. Optionally, the Gin recombinase catalytic domain variant included in the ZFR comprises a Phe104Asn and Ile94Val amino acid substitution with reference to a Gin recombinase catalytic domain comprising the amino acid as set forth in SEQ ID NO: 1. Optionally, the Gin recombinase catalytic domain variant included in the ZFR comprises a Ile94Val amino acid substitution with reference to a Gin recombinase catalytic domain comprising the amino acid as set forth in SEQ ID NO: 3.

In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence set forth in SEQ ID NO: 31. In some embodiments, the Gin recombinase catalytic domain variant included in the ZFR is a Gin recombinase catalytic domain comprising a Ile94Val amino acid substitution with reference to a Gin recombinase catalytic domain comprising the amino acid as set forth in SEQ ID NO: 31. In some embodiments, the Gin recombinase catalytic domain variant included in the ZFR is a Gin recombinase catalytic domain comprising a Phe104Asn and Ile94Val amino acid substitution with reference to a Gin recombinase catalytic domain comprising the amino acid as set forth in SEQ ID NO: 31. In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence set forth in SEQ ID NO: 32. In some embodiments, the Gin recombinase catalytic domain variant included in the ZFR is a Gin recombinase catalytic domain comprising a Ile94Val amino acid substitution with reference to a Gin recombinase catalytic domain comprising the amino acid as set forth in SEQ ID NO: 32. In some embodiments, the Gin recombinase catalytic domain variant included in the ZFR is a Gin recombinase catalytic domain comprising a Phe104Asn and Ile94Val amino acid substitution with reference to a Gin recombinase catalytic domain comprising the amino acid as set forth in SEQ ID NO: 32. In some embodiments, the Gin recombinase catalytic variant domain included in the ZFR is a Gin recombinase catalytic domain comprising a Phe104Asn amino acid substitution with reference to a Gin recombinase catalytic domain amino acid comprising the amino acid sequence set forth in SEQ ID NO: 33. In some embodiments, the Gin recombinase catalytic domain variant included in the ZFR is a Gin recombinase catalytic domain comprising a Ile94Val amino acid substitution with reference to a Gin recombinase catalytic domain comprising the amino acid as set forth in SEQ ID NO: 33 In some embodiments, the Gin recombinase catalytic domain variant included in the ZFR is a Gin recombinase catalytic domain comprising a Phe104Asn and Ile94Val amino acid substitution with reference to a Gin recombinase catalytic domain comprising the amino acid as set forth in SEQ ID NO: 33. In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence as set forth in SEQ ID NO: 34. In some embodiments, the Gin recombinase catalytic domain variant included in the ZFR is a Gin recombinase catalytic domain comprising a Ile94Val amino acid substitution with reference to a Gin recombinase catalytic domain comprising the amino acid as set forth in SEQ ID NO: 34. In some embodiments, the Gin recombinase catalytic domain variant included in the ZFR is a Gin recombinase catalytic domain comprising a Phe104Asn and Ile94Val amino acid substitution with reference to a Gin recombinase catalytic domain comprising the amino acid as set forth in SEQ ID NO: 34. In some embodiments, the Gin recombinase catalytic domain variant included in the ZFR is a Gin recombinase catalytic domain comprising a Phe104Asn amino acid substitution with reference to a Gin recombinase catalytic domain comprising the amino acid sequence as set forth in SEQ ID NO: 35. In some embodiments, the Gin recombinase catalytic domain variant included in the ZFR is a Gin recombinase catalytic domain comprising a Ile94Val amino acid substitution with reference to a Gin recombinase catalytic domain comprising the amino acid as set forth in SEQ ID NO: 35. In some embodiments, the Gin recombinase catalytic domain variant included in the ZFR is a Gin recombinase catalytic domain comprising a Phe104Asn and Ile94Val amino acid substitution with reference to a Gin recombinase catalytic domain comprising the amino acid as set forth in SEQ ID NO: 35.

In some embodiments, the Gin recombinase catalytic domain variant included in the ZFR is a Gin recombinase catalytic domain comprising a Phe104Asn amino acid substitution with reference to any one of sequences SEQ ID NO: 1, 31, 32, 33, 34 or 35, but no other “hyperactive” substitutions are present (e.g., D12G, N145, K50E, M70V, I94V and H106Y). In some embodiments, the Gin recombinase catalytic domain variant included in the ZFR is a Gin recombinase catalytic domain comprising a Phe104Asn amino acid substitution with reference to any one of sequences SEQ ID NO: 1, 31, 32, 33, 34 or 35, wherein D12G or H106Y substitutions are not present.

In some embodiments, the Gin recombinase catalytic domain variant included in the ZFR comprises the amino acid sequence set forth below (SEQ ID NO: 31):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERTMAGLAAARNKGRIGGRPPKSG

In some embodiments, the Gin recombinase catalytic domain variant included in the ZFR comprises the amino acid sequence set forth below (SEQ ID NO: 32):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERTMAGIAAARNKGRRFGRPPKSG

In some embodiments, the Gin recombinase catalytic domain variant included in the ZFR comprises the amino acid sequence set forth below (SEQ ID NO: 33):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERVMAGLAAARNKGRRFGRPPKSG

In some embodiments, the Gin recombinase catalytic domain variant included in the ZFR comprises the amino acid sequence set forth below (SEQ ID NO: 34):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRFFFHVMGALAEMERLSILERPMAGHAAARNKGRRFGRPPKSG

In some embodiments, the Gin recombinase catalytic domain variant included in the ZFR comprises the amino acid sequence set forth below (SEQ ID NO:35):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFHVMGALAEMERELIIERTSAGRAAAINKGRIMGRPRKSG

In some embodiments, the Gin recombinase catalytic domain variant included in the ZFR comprises the amino acid sequence set forth below (SEQ ID NO: 1):

MLIGYVRVST NDQNTDLQRN ALVCAGCEQI FEDKLSGTRT
DRPGLKRALK RLQKGDTLVV WKLDRLGRSM KHLISLVGEL
RERGINFRSL TDSIDTSSPM GRFFFHVMGA LAEMERELIL
ERVMAGIAAA RNKGRRWGRP P

In some embodiments, the Gin recombinase catalytic domain variant included in the ZFR comprises the amino acid sequence set forth below (SEQ ID NO: 2):

MLIGYVRVST NDQNTDLQRN ALVCAGCEQI FEDKLSGTRT
DRPGLKRALK RLQKGDTLVV WKLDRLGRSM KHLISLVGEL
RERGINFRSL TDSIDTSSPM GRFFFYVMGA LAEMERELIL
ERVMAGIAAA RNKGRRWGRP P

In some embodiments, the Gin recombinase catalytic domain variant included in the ZFR comprises the amino acid sequence set forth below (SEQ ID NO: 43):

MLIGYVRVSTNDQNTDLQRNALVCAGCEQIFEDKLSGTRTDRPGLKRA
LKRLQKGDTLVVWKLDRLGRSMKHLISLVGELRERGINFRSLTDSVDT
SSPMGRFFFHVMGALAEMERELILERVMAGIAAARNKGRRWGRPP

In some embodiments, the Gin recombinase catalytic domain variant included in the ZFR comprises the amino acid sequence set forth below (SEQ ID NO: 36):

MLIGYVRVST NDQNTDLQRN ALVCAGCEQI FEDKLSGTRT
DRPGLKRALK RLQKGDTLVV WKLDRLGRSM KHLISLVGEL
RERGINFRSL TDSIDTSSPM GRFNFYVMGA LAEMERELIL
ERVMAGIAAA RNKGRRWGRP P

In some embodiments, the Gin recombinase catalytic domain variant included in the ZFR comprises the amino acid sequence set forth below (SEQ ID NO: 42):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSVDTSS
PMGRFNFHVMGALAEMERELILERVMAGIAAARNKGRRWGRPP

In some embodiments, the Gin recombinase catalytic domain variant included in the ZFR comprises the amino acid sequence set forth below (SEQ ID NO: 37):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRFNEYVMGALAEMERELIIERTMAGLAAARNKGRIGGRPPKSG

In some embodiments, the Gin recombinase catalytic domain variant included in the ZFR comprises the amino acid sequence set forth below (SEQ ID NO: 44):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSVDTSS
PMGRENFHVMGALAEMERELIIERTMAGLAAARNKGRIGGRPPKSG

In some embodiments, the Gin recombinase catalytic domain variant included in the ZFR comprises the amino acid sequence set forth below (SEQ ID NO: 45):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRENFHVMGALAEMERELIIERTMAGLAAARNKGRIGGRPPKSG

In some embodiments, the Gin recombinase catalytic domain variant included in the ZFR comprises the amino acid sequence set forth below (SEQ ID NO: 46):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFYVMGALAEMERELIIERTMAGLAAARNKGRIGGRPPKSG

In some embodiments, the Gin recombinase catalytic domain variant included in the ZFR comprises the amino acid sequence set forth below (SEQ ID NO: 47):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSVDTSS
PMGREFFHVMGALAEMERELIIERTMAGLAAARNKGRIGGRPPKSG

In some embodiments, the Gin recombinase catalytic domain variant included in the ZFR comprises the amino acid sequence set forth below (SEQ ID NO: 38):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRFNEYVMGALAEMERELIIERTMAGIAAARNKGRRFGRPPKSG

In some embodiments, the Gin recombinase catalytic domain variant included in the ZFR comprises the amino acid sequence set forth below (SEQ ID NO: 48):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSVDTSS
PMGRENFHVMGALAEMERELIIERTMAGIAAARNKGRRFGRPPKSG

In some embodiments, the Gin recombinase catalytic domain variant included in the ZFR comprises the amino acid sequence set forth below (SEQ ID NO: 49):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRENFHVMGALAEMERELIIERTMAGIAAARNKGRRFGRPPKSG

In some embodiments, the Gin recombinase catalytic domain variant included in the ZFR comprises the amino acid sequence set forth below (SEQ ID NO: 50):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGREFFYVMGALAEMERELIIERTMAGIAAARNKGRRFGRPPKSG

In some embodiments, the Gin recombinase catalytic domain variant included in the ZFR comprises the amino acid sequence set forth below (SEQ ID NO: 51):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSVDTSS
PMGREFFHVMGALAEMERELIIERTMAGIAAARNKGRRFGRPPKSG

In some embodiments, the Gin recombinase catalytic domain variant included in the ZFR comprises the amino acid sequence set forth below (SEQ ID NO: 39):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRFNEYVMGALAEMERELIIERVMAGLAAARNKGRRFGRPPKSG

In some embodiments, the Gin recombinase catalytic domain variant included in the ZFR comprises the amino acid sequence set forth below (SEQ ID NO: 52):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSVDTSS
PMGRENFHVMGALAEMERELIIERVMAGLAAARNKGRREGRPPKSG

In some embodiments, the Gin recombinase catalytic domain variant included in the ZFR comprises the amino acid sequence set forth below (SEQ ID NO: 53):

MLIGYVRVSTNDQNTDLQRNALVCAGCEQIFEDKLSGTRTDRPGLKRAL
KRLQKGDTLVVWKLDRLGRSMKHLISLVGELRERGINFRSLTDSIDTSS
PMGRENFHVMGALAEMERELIIERVMAGLAAARNKGRRFGRPPKSG

In some embodiments, the Gin recombinase catalytic domain variant included in the ZFR comprises the amino acid sequence set forth below (SEQ ID NO: 54):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GREFFYVMGALAEMERELIIERVMAGLAAARNKGRRFGRPPKSG

In some embodiments, the Gin recombinase catalytic domain variant included in the ZFR comprises the amino acid sequence set forth below (SEQ ID NO: 55):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSVDTSSPM
GREFFHVMGALAEMERELIIERVMAGLAAARNKGRRFGRPPKSG

In some embodiments, the Gin recombinase catalytic domain variant included in the ZFR comprises the amino acid sequence set forth below (SEQ ID NO: 40):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFFFYVMGALAEMERLSILERPMAGHAAARNKGRRFGRPPKSG

In some embodiments, the Gin recombinase catalytic domain variant included in the ZFR comprises the amino acid sequence set forth below (SEQ ID NO: 56):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFNFYVMGALAEMERLSILERPMAGHAAARNKGRRFGRPPKSG

In some embodiments, the Gin recombinase catalytic domain variant included in the ZFR comprises the amino acid sequence set forth below (SEQ ID NO: 57):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSVDTSSPM
GRFNFHVMGALAEMERLSILERPMAGHAAARNKGRRFGRPPKSG

In some embodiments, the Gin recombinase catalytic domain variant included in the ZFR comprises the amino acid sequence set forth below (SEQ ID NO: 58):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFNFHVMGALAEMERLSILERPMAGHAAARNKGRRFGRPPKSG

In some embodiments, the Gin recombinase catalytic domain variant included in the ZFR comprises the amino acid sequence set forth below (SEQ ID NO: 59):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSVDTSSPM
GRFFFHVMGALAEMERLSILERPMAGHAAARNKGRRFGRPPKSG

In some embodiments, the Gin recombinase catalytic domain variant included in the ZFR comprises the amino acid sequence set forth below (SEQ ID NO: 41):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GREFFYVMGALAEMERELIIERTSAGRAAAINKGRIMGRPRKSG

In some embodiments, the Gin recombinase catalytic domain variant included in the ZFR comprises the amino acid sequence set forth below (SEQ ID NO: 60):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFNEYVMGALAEMERELIIERTSAGRAAAINKGRIMGRPRKSG

In some embodiments, the Gin recombinase catalytic domain variant included in the ZFR comprises the amino acid sequence set forth below (SEQ ID NO: 61):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSVDTSSPM
GRENFHVMGALAEMERELIIERTSAGRAAAINKGRIMGRPRKSG

In some embodiments, the Gin recombinase catalytic domain variant included in the ZFR comprises the amino acid sequence set forth below (SEQ ID NO: 62):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRENFHVMGALAEMERELIIERTSAGRAAAINKGRIMGRPRKSG

In some embodiments, the Gin recombinase catalytic domain variant included in the ZFR comprises the amino acid sequence set forth below (SEQ ID NO: 63):

MLIGYVRVSTNDQNTDLQRNALVCAGCEQIFEDKLSGTRTDRPGLKRALK
RLQKGDTLVVWKLDRLGRSMKHLISLVGELRERGINFRSLTDSVDTSSPM
GREFFHVMGALAEMERELIIERTSAGRAAAINKGRIMGRPRKSG

In some embodiments, the Gin recombinase catalytic domain variant included in the ZFR comprising the Phe104Asn mutation comprises the sequence set forth below (SEQ ID NO: 3):

MLIGYVRVST NDQNTDLQRN ALVCAGCEQI FEDKLSGTRT
DRPGLKRALK RLQKGDTLVV WKLDRLGRSM KHLISLVGEL
RERGINFRSL TDSIDTSSPM GRFNFHVMGA LAEMERELIL
ERVMAGIAAA RNKGRRWGRP P

In some embodiments, the Gin recombinase catalytic domain variant included in the ZFR comprises the amino acid sequence set forth in SEQ ID NO: 3 (which comprises Phe104Asn substitution with respect to the Gin recombinase catalytic domain of SEQ ID NO: 1)). In some embodiments, the Gin recombinase catalytic domain variant included in the ZFR comprises an amino acid sequence having at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference SEQ ID NO: 3, as determined by sequence alignment programs known by skilled artisans. In some embodiments, the Gin recombinase catalytic domain variant included in the ZFR comprises the amino acid sequence set forth in SEQ ID NO: 3, which comprises Phe104Asn substitution with respect to the Gin recombinase catalytic domain (SEQ ID NO: 1). In some embodiments, the Gin recombinase catalytic domain variant included in the ZFR comprise the amino acid sequence set forth in SEQ ID NO: 3 and does not comprise other hyperactive mutations, such as D12G or H106Y.

In some embodiments, the zinc finger proteins used in the zinc-finger recombinases comprise a left arm. In some embodiments, the zinc finger proteins used in the zinc-finger recombinases comprise a right arm. In some embodiments, the zinc finger proteins used in the zinc-finger recombinases comprise a left arm and a right arm.

In some embodiments, the polynucleotide sequence encoding the Gin recombinase catalytic domain variant included in the ZFR comprises the sequence set forth in SEQ ID NO: 6 below:

atg ctg atc ggc tat gtg cgg gtg tcc acc aac gac
cag aac acc gac ctg cag aga aac gcc ctt gtg tgt
gcc gga tgc gag cag atc ttc gag gat aag ctg agc
ggc acc aga acc gac aga ccc gga ctg aag aga gcc
ctg aag aga ctg cag aaa ggc gac acc ctg gtc gtg
tgg aag ctg gat aga ctg ggc cgc agc atg aag cac
ctg atc agc ctt gtg ggc gag ctg aga gag cgg ggc
atc aac ttt aga agc ctg acc gac agc atc gac aca
agc agc cct atg ggc aga ttc ttc ttc tac gtg atg
ggc gcc ctg gcc gag atg gaa aga gag ctg atc ctg
gag aga gtc atg gcc gga atc gcc gct gcc aga aac
aag gga aga aga tgg ggc cgg cca cct aag tct ggc
aca ggc gag agg ccc ttc cag tgt cga atc tgc atg

In some embodiments, the polynucleotide sequence encoding the Gin recombinase catalytic domain variant included in the ZFR comprises the sequence set forth in SEQ ID NO: 7 below, which encodes the Gin recombinase catalytic domain comprising a Phe104Asn substitution with respect to the Gin recombinase catalytic domain (SEQ ID NO: 6):

atg ctg atc ggc tat gtg cgg gtg tcc acc aac gac
cag aac acc gac ctg cag aga aac gcc ctt gtg tgt
gcc gga tgc gag cag atc ttc gag gat aag ctg agc
ggc acc aga acc gac aga ccc gga ctg aag aga gcc
ctg aag aga ctg cag aaa ggc gac acc ctg gtc gtg
tgg aag ctg gat aga ctg ggc cgc agc atg aag cac
ctg atc agc ctt gtg ggc gag ctg aga gag cgg ggc
atc aac ttt aga agc ctg acc gac agc atc gac aca
agc agc cct atg ggc aga ttc aac ttc cat gtg atg
ggc gcc ctg gcc gag atg aga aga tgg ggc cgg cca
cct

In some embodiments, the Gin recombinase nucleotide sequence included in the nucleotide sequence encoding a ZFR of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference SEQ ID NO: 7, as determined by sequence alignment programs known by skilled artisans.

In some embodiments, the catalytic domain of the Gin recombinase protein is operably linked to a zinc-finger nucleotide binding domain. Optionally, the catalytic domain of the Gin recombinase protein is connected by way of a linker to the zinc-finger nucleotide binding domain, wherein said recombinase is capable of binding a target nucleic acid sequence by way of said zinc-finger nucleotide binding domain. In some embodiments, the linker region is a sequence with structural flexibility. In some embodiments, the linker region comprises at least 3, at least 6, at least 10, at least 20, at least 30 or at least 40 amino acids in length. Preferably, the nucleic acid is 3, 4, 6, 9, 12, 15, 18, 21, 24, 27, 30, 33, 36, 39, 42 or 45 amino acids in length. In some embodiments, the sequence of the linker is the sequence set forth in SEQ ID NO: 8 (KSGTG). In some embodiments, a 3×FLAG (DYKDHDGDYKDHDIDYKDDDDK; SEQ ID NO: 18) and SV40 NLS (PKKKRKV; SEQ ID NO: 19) sequences are included upstream of the γ Gin recombinase protein sequence.

In some embodiments, the polynucleotide encoding the zinc-finger recombinase protein comprises a polynucleotide encoding the catalytic domain of Gin recombinase protein operably linked to a zinc-finger nucleotide binding domain. Optionally, the polynucleotide encoding the catalytic domain of the Gin recombinase protein is connected by way of a linker to the zinc-finger nucleotide binding domain. In some embodiments, the linker comprises at least 6, at least 10, at least 20, at least 30 or at least 40 base pairs in length. Preferably, the nucleic acid is 6, 9, 12, 15, 18, 21, 24, 27, 30, 33, 36, 39, 42 or 45 base pairs in length.

A “zinc finger protein” or “ZFP” refers to a protein having zinc finger nucleotide binding domains that are stabilized by zinc. ZFPs bind to DNA in a sequence-specific manner. The individual DNA-binding domains are referred to as “fingers.” A ZFP has at least one finger, each finger binds from two to four base pairs of DNA, typically three or four base pairs of DNA. Each zinc finger typically comprises approximately 30 amino acids and chelates zinc. A ZFP may be engineered to have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers that bind the particular triplet or quadruplet sequence. See, e.g., ZFP design methods described in detail in U.S. Pat. Nos. 5,789,538; 5,925,523; 6,007,988; 6,013,453; 6,140,081; 6,200,759; 6,453,242; 6,534,261; 6,979,539; and 8,586,526; and International Patent Publications WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084; and WO 03/016496.

Zinc finger nucleotide binding domains (ZFPs) include at least one zinc finger but can include a plurality of zinc fingers (e.g., 2, 3, 4, 5, 6 or more fingers). Usually, the ZFPs include at least three fingers. Certain of the ZFPs include four, five or six fingers, while some ZFPs include 8, 9, 10, 11 or 12 or more fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more functional (regulatory) domains, which domains can be transcriptional activation or repression domains or other domains such as DNMT domains. The DNA binding domains fused to at least one regulatory (functional) domain and can be thought of as a ‘ZFP-TF’ architecture. Selection of target sites; ZFPs and methods for design and construction of zinc-finger recombinase proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; and International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

Methods and compositions can also be used to increase the specificity of a ZFP for its intended target relative to other unintended cleavage sites, known as off-target sites for example by mutations to the ZFP backbone as described in U.S. Patent Publication No. 20180087072. Thus, zinc finger nucleotide binding domains described herein can comprise mutations in one or more of their DNA binding domain backbone regions. These ZFPs can include mutations to amino acid within the ZFP DNA binding domain (‘ZFP backbone’) that can interact non-specifically with phosphates on the DNA backbone, but they do not comprise changes in the DNA recognition helices. Thus, the invention includes mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In some embodiments, mutations at made at position (−5), (−9) and/or position (−14) relative to the DNA binding helix. In some embodiments, a zinc finger may comprise one or more mutations at (−5), (−9) and/or (−14). In further embodiments, one or more zinc finger in a multi-finger zinc finger protein may comprise mutations in (−5), (−9) and/or (−14). In some embodiments, the amino acids at (−5), (−9) and/or (−14) (e.g. an arginine (R) or lysine (K)) are mutated to an alanine (A), leucine (L), Ser (S), Asp (N), Glu (E), Tyr (Y) and/or glutamine (Q).

In some embodiments, the left arm of the zinc finger nucleotide binding domain comprises the amino acid sequence set forth in SEQ ID NO: 9 below:

ERPFQCRICMRNFSRPYTLRLHIRTHTGEKPFACDICGRKFADNSNRIK
HTKIHTHPRAPIPKPFQCRICMRNFSQSSDLSRHIRTHTGEKPFACDIC
GRKFAQSGNLARHTKIHTGSQKPFQCRICMRNFSRQDCLSLHIRTHTGE
KPFACDICGRKFARNDNRKTHTKIHLRQKD

In some embodiments, the right arm of zinc finger nucleotide binding domain comprises the amino acid sequence set forth in SEQ ID NO: 10 below:

ERPFQCRICMRNFSLRHHLTRHIRTHTGEKPFACDICGRKFAQSYARTL
HTKIHTHPRAPIPKPFQCRICMRNFSWRSSLKTHIRTHTGEKPFACDIC
GRKFADRSNRKTHTKIHTHPRAPIPKPFQCRICMRNFSDRSNLSRHIRT
HTGEKPFACDICGRKFAQSGNLARHTKIHLRQKD

In some embodiments, the amino acid sequence of the left arm of the zinc-finger recombinase protein of the disclosure comprises the sequence set forth below (SEQ ID NO: 64), where the Gin recombinase F104N variant catalytic domain amino acid sequence is depicted in bold, the zinc-finger protein amino acid sequence is depicted in italics and the linker is underlined.

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFNFHVMGALAEMERELILERVMAGIAAARNKGRRWGRPPKSGTGERPF
QCRICMRNFSRPYTLRLHIRTHTGEKPFACDICGRKFADNSNRIKHTKIH
THPRAPIPKPFQCRICMRNFSQSSDLSRHIRTHTGEKPFACDICGRKFAQ
SGNLARHTKIHTGSQKPFQCRICMRNFSRQDCLSLHIRTHTGEKPFACDI
CGRKFARNDNRKTHTKIHLRQKD

In some embodiments, the amino acid sequence of the right arm of the zinc-finger recombinase protein of the disclosure is the sequence set forth below (SEQ ID NO: 65), where the Gin recombinase F104N variant catalytic domain amino acid sequence is depicted in bold, the zinc-finger protein amino acid sequence is depicted in italics and the linker is underlined.

MLIGYVRVSTNDQNTDLQRNALVCAGCEQIFEDKLSGTRTDRPGLKRALK
RLQKGDTLVVWKLDRLGRSMKHLISLVGELRERGINFRSLTDSIDTSSPM
GRFNFHVMGALAEMERELILERVMAGIAAARNKGRRWGRPPKSGTGERPF
QCRICMRNFSLRHHLTRHIRTHTGEKPFACDICGRKFAQSYARTLHTKIH
THPRAPIPKPFQCRICMRNFSWRSSLKTHIRTHTGEKPFACDICGRKFAD
RSNRKTHTKIHTHPRAPIPKPFQCRICMRNFSDRSNLSRHIRTHTGEKPF
ACDICGRKFAQSGNLARHTKIHLRQKD

In some embodiments, the amino acid sequence of the left arm of the zinc-finger recombinase protein of the disclosure comprises the sequence set forth below (SEQ ID NO: 4), where the Gin recombinase F104N variant catalytic domain amino acid sequence is depicted in bold, the zinc-finger protein amino acid sequence is depicted in italics and the linker is underlined. In some embodiments, a 3×FLAG (DYKDHDGDYKDHDIDYKDDDDK; SEQ ID NO: 18) and SV40 NLS (PKKKRKV; SEQ ID NO: 19) sequences are included upstream of the γ Gin recombinase protein sequence.

MDYKDHDGDYKDHDIDYKDDDDKAPKKKRKVDMLIGYVRVSTNDQNTDLQ
RNALVCAGCEQIFEDKLSGTRTDRPGLKRALKRLQKGDTLVVWKLDRLGR
SMKHLISLVGELRERGINFRSLTDSIDTSSPMGRFNFHVMGALAEMEREL
ILERVMAGIAAARNKGRRWGRPPKSGTGERPFQCRICMRNFSRPYTLRLH
IRTHTGEKPFACDICGRKFADNSNRIKHTKIHTHPRAPIPKPFQCRICMR
NFSQSSDLSRHIRTHTGEKPFACDICGRKFAQSGNLARHTKIHTGSQKPF
QCRICMRNFSRQDCLSLHIRTHTGEKPFACDICGRKFARNDNRKTHTKIH
LRQKD

In some embodiments, the amino acid sequence of the right arm of the zinc-finger recombinase protein of the disclosure is the sequence set forth below (SEQ ID NO: 5), where the Gin recombinase F104N variant catalytic domain amino acid sequence is depicted in bold, the zinc-finger protein amino acid sequence is depicted in italics and the linker is underlined. In some embodiments, a 3×FLAG (DYKDHDGDYKDHDIDYKDDDDK; SEQ ID NO: 18) and SV40 NLS (PKKKRKV; SEQ ID NO: 19) sequences are included upstream of the γ Gin recombinase protein sequence.

MDYKDHDGDYKDHDIDYKDDDDKAPKKKRKVDMLIGYVRVSTNDQNTDLQ
RNALVCAGCEQIFEDKLSGTRTDRPGLKRALKRLQKGDTLVVWKLDRLGR
SMKHLISLVGELRERGINFRSLTDSIDTSSPMGRFNFHVMGALAEMEREL
ILERVMAGIAAARNKGRRWGRPPKSGTGERPFQCRICMRNFSLRHHLTRH
IRTHTGEKPFACDICGRKFAQSYARTLHTKIHTHPRAPIPKPFQCRICMR
NFSWRSSLKTHIRTHTGEKPFACDICGRKFADRSNRKTHTKIHTHPRAPI
PKPFQCRICMRNFSDRSNLSRHIRTHTGEKPFACDICGRKFAQSGNLARH
TKIHLRQKD

In some embodiments, the zinc finger nucleotide binding domain binds an endogenous locus. In some embodiments, the endogenous locus is selected from the group consisting of Hypoxanthine-guanine phosphoribosyltransferase (HPRT) gene, T Cell Receptor Alpha Constant (TRAC) and a safe-harbor locus. In some embodiments, the endogenous locus is selected from the group consisting of Hypoxanthine-guanine phosphoribosyltransferase (HPRT) gene, T Cell Receptor Alpha Constant (TRAC), Adeno-Associated Virus Integration Site 1 (AAVS1) and a safe-harbor locus. In some embodiments, the safe-harbor locus is in chromosome 1.

In some embodiments, the zinc finger nucleotide binding domain binds an HPRT gene. In some embodiments, the HPRT zinc finger recombinase protein donor plasmid comprises the nucleotide sequence set forth in SEQ ID NO: 11:

AGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTC
CAACCCGGTAAGACACGACTTATCGCCACTGGCAGCAGCCACTGGTAAC
AGGATTAGCAGAGCGAGGTATGTAGGCGGTGCTACAGAGTTCTTGAAGT
GGTGGCCTAACTACGGCTACACTAGAAGAACAGTATTTGGTATCTGCGC
TCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGC
AGATTACGCGCAGAAAAAAAGGATCTCAAGAAGATCCTTTGATCTTTTC
TACGGGGTCTGACGCTCAGTGGAACGAAAACTCACGTTAAGGGATTTTG
GTCATGAGATTATCAAAAAGGATCTTCACCTAGATCCTTTTAAATTAAA
AATGAAGTTTTAAATCAATCTAAAGTATATATGAGTAAACTTGGTCTGA
CAGTTACCAATGCTTAATCAGTGAGGCACCTATCTCAGCGATCTGTCTA
TTTCGTTCATCCATAGTTGCCTGACTCCCCGTCGTGTAGATAACTACGA
TACGGGAGGGCTTACCATCTGGCCCCAGTGCTGCAATGATACCGCGAGA
CCCACGCTCACCGGCTCCAGATTTATCAGCAATAAACCAGCCAGCCGGA
AGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAG
TTTGCGCAACGTTGTTGCCATTGCTACAGGCATCGTGGTGTCACGCTCG
TCGTTTGGTATGGCTTCATTCAGCTCCGGTTCCCAACGATCAAGGCGAG
TTACATGATCCCCCATGTTGTGCAAAAAAGCGGTTAGCTCCTTCGGTCC
TCCGATCGTTGTCAGAAGTAAGTTGGCCGCAGTGTTATCACTCATGGTT
ATGGCAGCACTGCATAATTCTCTTACTGTCATGCCATCCGTAAGATGCT
TTTCTGTGACTGGTGAGTACTCAACCAAGTCATTCTGAGAATAGTGTAT
GCGGCGACCGAGTTGCTCTTGCCCGGCGTCAATACGGGATAATACCGCG
CCACATAGCAGAACTTTAAAAGTGCTCATCATTGGAAAACGTTCTTCGG
GGCGAAAACTCTCAAGGATCTTACCGCTGTTGAGATCCAGTTCGATGTA
ACCCACTCGTGCACCCAACTGATCTTCAGCATCTTTTACTTTCACCAGC
GTTTCTGGGTGAGCAAAAACAGGAAGGCAAAATGCCGCAAAAAAGGGAA
TAAGGGCGACACGGAAATGTTGAATACTCATACTCTTCCTTTTTCAATA
TTATTGAAGCATTTATCAGGGTTATTGTCTCATGAGCGGATACATATTT
GAATGTATTTAGAAAAATAAACAAATAGGGGTTCCGCGCACATTTCCCC
GAAAAGTGCCACCTGACGTCTAAGAAACCATTATTATCATGACATTAAC
CTATAAAAATAGGCGTATCACGAGGCCCTTTCGTCTCGCGCGTTTCGGT
GATGACGGTGAAAACCTCTGACACATGCAGCTCCCGGAGACGGTCACAG
CTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAG
CAGATTGTACTGAGAGTGCACCATATGCGGTGTGAAATACCGCACAGAT
GCGTAAGGAGAAAATACCGCATCAGGCGCCATTCGCCATTCAGGCTGCG
CAACTGTTGGGAAGGGCGATCGGTGCGGGCCTCTTCGCTATTACGCCAG
CTGGCGAAAGGGGGATGTGCTGCAAGGCGATTAAGTTGGGTAACGCCAG
GGTTTTCCCAGTCACGACGTTGTAAAACGACGGCCAGTGAATTCATGAA
AAGGTGGAAGCTTTACTTGGTTATAACTTTACTTTTAATACCTAGAACA
GTGAGTCTTCAAACTGCTTTATTGATGCCCAATTTATAAAAAGTTTCCT
GAGCATTTACCCCTAATATATGCTCTGCTAATGGAGCTATGCAATCTAC
TGAATGTCATAATCAACTTCAAGGATATACTGAAGCTATGTGGGCTTCT
CATGAAAGAGAATTTTTTGCTTGTAAGGAAAGATGTATGCTTGAAATAA
GCCACCATAGAGACCCATGTCCTAACAGCTCCCTGGAGACGGCTAATGA
TACTTGGAAGCTTGGCGTAATCATGGTCATAGCTGTTTCCTGTGTGAAA
TTGTTATCCGCTCACAATTCCACACAACATACGAGCCGGAAGCATAAAG
TGTAAAGCCTGGGGTGCCTAATGAGTGAGCTAACTCACATTAATTGCGT
TGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGC
GGCGAGCGGTATCAGCTCACTCAAAGGCGGTAATACGGTTATCCACAGA
ATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCC
GCCCCCCTGACGAGCATCACAAAAATCGACGCTCAAGTCAGAGGTGGCG
AAACCCGACAGGACTATAAAGATACCAGGCGTTTCCCCCTGGAAGCTCC
CTCGTGCGCTCTCCTGTTCCGACCCTGCCGCTTACCGGATACCTGTCCG
CCTTTCTCCCTTCGGGAAGCGTGGCGCTTTCTCATAGCTCACGCTGTAG
GTATCTC

The zinc-finger recombinase protein promotes site-specific recombination between DNA targets that consist of two zinc-finger binding sites flanking a central 20-bp core sequence recognized by the recombinase catalytic domain. In some embodiments, the 20-bp core sequence recognized by the Gin recombinase catalytic domain of the disclosure comprises the nucleotide sequence set forth in SEQ ID NO: 12: GTTATAACTTTACTTTTAAT. In some embodiments, the two zinc-finger binding sites comprise the nucleotide sequence set forth in SEQ ID NO: 13 (AAGGTGGAAGCTTTACTTG) and SEQ ID NO: 14 (ACCTAGAACAGTGAGTCTTC), respectively. In some embodiments, the full target site of the zinc-finger recombinase protein comprises the nucleotide sequence set forth in SEQ ID NO: 15:

AAGGTGGAAGCTTTACTTGGTTATAACTTTACTTTTAATACCTAGAACA
GTGAGTCTTC

In some embodiments, the disclosure provides a pVAX plasmid comprising a polynucleotide encoding the left arm of a zinc finger recombinase protein. In some embodiments, the nucleotide sequence of the plasmid encoding the left arm of a zinc-finger recombinase protein comprises the sequence set forth in SEQ ID NO: 16:

CTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGAT
GCGGTGGGCTCTATGGCTTCTACTGGGCGGTTTTATGGACAGCAAGCGAA
CCGGAATTGCCAGCTGGGGCGCCCTCTGGTAAGGTTGGGAAGCCCTGCAA
AGTAAACTGGATGGCTTTCTCGCCGCCAAGGATCTGATGGCGCAGGGGAT
CAAGCTCTGATCAAGAGACAGGATGAGGATCGTTTCGCATGATTGAACAA
GATGGATTGCACGCAGGTTCTCCGGCCGCTTGGGTGGAGAGGCTATTCGG
CTATGACTGGGCACAACAGACAATCGGCTGCTCTGATGCCGCCGTGTTCC
GGCTGTCAGCGCAGGGGCGCCCGGTTCTTTTTGTCAAGACCGACCTGTCC
GGTGCCCTGAATGAACTGCAAGACGAGGCAGCGCGGCTATCGTGGCTGGC
CACGACGGGCGTTCCTTGCGCAGCTGTGCTCGACGTTGTCACTGAAGCGG
GAAGGGACTGGCTGCTATTGGGCGAAGTGCCGGGGCAGGATCTCCTGTCA
TCTCACCTTGCTCCTGCCGAGAAAGTATCCATCATGGCTGATGCAATGCG
GCGGCTGCATACGCTTGATCCGGCTACCTGCCCATTCGACCACCAAGCGA
AACATCGCATCGAGCGAGCACGTACTCGGATGGAAGCCGGTCTTGTCGAT
CAGGATGATCTGGACGAAGAGCATCAGGGGCTCGCGCCAGCCGAACTGTT
CGCCAGGCTCAAGGCGAGCATGCCCGACGGCGAGGATCTCGTCGTGACCC
ATGGCGATGCCTGCTTGCCGAATATCATGGTGGAAAATGGCCGCTTTTCT
GGATTCATCGACTGTGGCCGGCTGGGTGTGGCGGACCGCTATCAGGACAT
AGCGTTGGCTACCCGTGATATTGCTGAAGAGCTTGGCGGCGAATGGGCTG
ACCGCTTCCTCGTGCTTTACGGTATCGCCGCTCCCGATTCGCAGCGCATC
GCCTTCTATCGCCTTCTTGACGAGTTCTTCTGAATTATTAACGCTTACAA
TTTCCTGATGCGGTATTTTCTCCTTACGCATCTGTGCGGTATTTCACACC
GCATACAGGTGGCACTTTTCGGGGAAATGTGCGCGGAACCCCTATTTGTT
TATTTTTCTAAATACATTCAAATATGTATCCGCTCATGAGACAATAACCC
TGATAAATGCTTCAATAATAGCACGTGCTAAAACTTCATTTTTAATTTAA
AAGGATCTAGGTGAAGATCCTTTTTGATAATCTCATGACCAAAATCCCTT
AACGTGAGTTTTCGTTCCACTGAGCGTCAGACCCCGTAGAAAAGATCAAA
GGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTAC
CAACTCTTTTTCCGAAGGTAACTGGCTTCAGCAGAGCGCAGATACCAAAT
ACTGTCCTTCTAGTGTAGCCGTAGTTAGGCCACCACTTCAAGAACTCTGT
AGCACCGCCTACATACCTCGCTCTGCTAATCCTGTTACCAGTGGCTGCTG
CCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCC
CAGCTTGGAGCGAACGACCTACACCGAACTGAGATACCTACAGCGTGAGC
TATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGG
AAACGCCTGGTATCTTTATAGTCCTGTCGGGTTTCGCCACCTCTGACTTG
AGCGTCGATTTTTGTGATGCTCGTCAGGGGGGCGGAGCCTATGGAAAAAC
GCCAGCAACGCGGCCTTTTTACGGTTCCTGGGCTTTTGCTGGCCTTTTGC
TCACATGTTCTTGACTCTTCGCGATGTACGGGCCAGATATACGCGTTGAC
ATTGATTATTGACTAGTTATTAATAGTAATCAATTACGGGGTCATTAGTT
CATAGCCCATATATGGAGTTCCGCGTTACATAACTTACGGTAAATGGCCC
GCCTGGCTGACCGCCCAACGACCCCCGCCCATTGACGTCAATAATGACGT
ATGTTCCCATAGTAACGCCAATAGGGACTTTCCATTGACGTCAATGGGTG
GACTATTTACGGTAAACTGCCCACTTGGCAGTACATCAAGTGTATCATAT
GCCAAGTACGCCCCCTATTGACGTCAATGACGGTAAATGGCCCGCCTGGC
ATTATGCCCAGTACATGACCTTATGGGACTTTCCTACTTGGCAGTACATC
TACGTATTAGTCATCGCTATTACCATGGTGATGCGGTTTTGGCAGTACAT
CAATGGGCGTGGATAGCGGTTTGACTCACGGGGATTTCCAAGTCTCCACC
CCATTGACGTCAATGGGAGTTTGTTTTGGCACCAAAATCAACGGGACTTT
CCAAAATGTCGTAACAACTCCGCCCCATTGACGCAAATGGGCGGTAGGCG
TGTACGGTGGGAGGTCTATATAAGCAGAGCTCTCTGGCTAACTAGAGAAC
CCACTGCTTACTGGCTTATCGAAATTAATACGACTCACTATAGGGAGAGC
CAAGCTGACTAGCGTTTAAACTTAAGCTGATCCACTAGTCCAGTGTGGTG
GAATTCGCCACCATGGACTACAAAGACCACGACGGTGATTATAAAGATCA
CGACATCGATTACAAGGACGACGACGACAAGGCCCCCAAGAAGAAGAGGA
AGGTCGACATGCTGATCGGCTATGTGCGGGTGTCCACCAACGACCAGAAC
ACCGACCTGCAGAGAAACGCCCTTGTGTGTGCCGGATGCGAGCAGATCTT
CGAGGATAAGCTGAGCGGCACCAGAACCGACAGACCCGGACTGAAGAGAG
CCCTGAAGAGACTGCAGAAAGGCGACACCCTGGTCGTGTGGAAGCTGGAT
AGACTGGGCCGCAGCATGAAGCACCTGATCAGCCTTGTGGGCGAGCTGAG
AGAGCGGGGCATCAACTTTAGAAGCCTGACCGACAGCATCGACACAAGCA
GCCCTATGGGCAGATTCAACTTCCATGTGATGGGCGCCCTGGCCGAGATG
GAAAGAGAGCTGATCCTGGAGAGAGTCATGGCCGGAATCGCCGCTGCCAG
AAACAAGGGAAGAAGATGGGGCCGGCCACCTAAGTCTGGCACAGGCGAGA
GGCCCTTCCAGTGTCGAATCTGCATGCGTAACTTCAGTCGCCCGTACACC
CTGCGCCTGCACATCCGCACCCACACCGGCGAGAAGCCTTTTGCCTGTGA
CATTTGTGGGAGGAAATTTGCCGACAACTCCAACCGCATCAAGCATACCA
AGATACACACGCATCCCAGGGCACCTATTCCCAAGCCCTTCCAGTGTCGA
ATCTGCATGCGTAACTTCAGTCAGTCCTCCGACCTGTCCCGCCACATCCG
CACCCACACCGGCGAGAAGCCTTTTGCCTGTGACATTTGTGGGAGGAAAT
TTGCCCAGTCCGGCAACCTGGCCCGCCATACCAAGATACACACGGGATCT
CAGAAGCCCTTCCAGTGTCGAATCTGCATGCGTAACTTCAGTCGCCAGGA
CTGTCTGTCCCTGCACATCCGCACCCACACCGGCGAGAAGCCTTTTGCCT
GTGACATTTGTGGGAGGAAATTTGCCCGCAACGACAACCGCAAGACCCAT
ACCAAGATACACCTGCGGCAGAAGGACTGATAAC

In some embodiments, the disclosure provides a pVAX plasmid comprising a polynucleotide encoding the right arm of a zinc finger recombinase protein. In some embodiments, the nucleotide sequence of the plasmid encoding the right arm of a zinc-finger recombinase protein comprises the sequence set forth in SEQ ID NO: 17:

GGAGGATTGGGAAGACAATAGCAGGCATGCTGGGGATGCGGTGGGCTCTA
TGGCTTCTACTGGGCGGTTTTATGGACAGCAAGCGAACCGGAATTGCCAG
CTGGGGCGCCCTCTGGTAAGGTTGGGAAGCCCTGCAAAGTAAACTGGATG
GCTTTCTCGCCGCCAAGGATCTGATGGCGCAGGGGATCAAGCTCTGATCA
AGAGACAGGATGAGGATCGTTTCGCATGATTGAACAAGATGGATTGCACG
CAGGTTCTCCGGCCGCTTGGGTGGAGAGGCTATTCGGCTATGACTGGGCA
CAACAGACAATCGGCTGCTCTGATGCCGCCGTGTTCCGGCTGTCAGCGCA
GGGGCGCCCGGTTCTTTTTGTCAAGACCGACCTGTCCGGTGCCCTGAATG
AACTGCAAGACGAGGCAGCGCGGCTATCGTGGCTGGCCACGACGGGCGTT
CCTTGCGCAGCTGTGCTCGACGTTGTCACTGAAGCGGGAAGGGACTGGCT
GCTATTGGGCGAAGTGCCGGGGCAGGATCTCCTGTCATCTCACCTTGCTC
CTGCCGAGAAAGTATCCATCATGGCTGATGCAATGCGGCGGCTGCATACG
CTTGATCCGGCTACCTGCCCATTCGACCACCAAGCGAAACATCGCATCGA
GCGAGCACGTACTCGGATGGAAGCCGGTCTTGTCGATCAGGATGATCTGG
ACGAAGAGCATCAGGGGCTCGCGCCAGCCGAACTGTTCGCCAGGCTCAAG
GCGAGCATGCCCGACGGCGAGGATCTCGTCGTGACCCATGGCGATGCCTG
CTTGCCGAATATCATGGTGGAAAATGGCCGCTTTTCTGGATTCATCGACT
GTGGCCGGCTGGGTGTGGCGGACCGCTATCAGGACATAGCGTTGGCTACC
CGTGATATTGCTGAAGAGCTTGGCGGCGAATGGGCTGACCGCTTCCTCGT
GCTTTACGGTATCGCCGCTCCCGATTCGCAGCGCATCGCCTTCTATCGCC
TTCTTGACGAGTTCTTCTGAATTATTAACGCTTACAATTTCCTGATGCGG
TATTTTCTCCTTACGCATCTGTGCGGTATTTCACACCGCATACAGGTGGC
ACTTTTCGGGGAAATGTGCGCGGAACCCCTATTTGTTTATTTTTCTAAAT
ACATTCAAATATGTATCCGCTCATGAGACAATAACCCTGATAAATGCTTC
AATAATAGCACGTGCTAAAACTTCATTTTTAATTTAAAAGGATCTAGGTG
AAGATCCTTTTTGATAATCTCATGACCAAAATCCCTTAACGTGAGTTTTC
GTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCC
GAAGGTAACTGGCTTCAGCAGAGCGCAGATACCAAATACTGTCCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGGCTTTTGCTGGCCTTTTGCTCACATGTTCTTG
ACTCTTCGCGATGTACGGGCCAGATATACGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGACTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCTGGCTAACTAGAGAACCCACTGCTTACTG
GCTTATCGAAATTAATACGACTCACTATAGGGAGAGCCAAGCTGACTAGC
GTTTAAACTTAAGCTGATCCACTAGTCCAGTGTGGTGGAATTCGCCACCA
TGGACTACAAAGACCACGACGGTGATTATAAAGATCACGACATCGATTAC
AAGGACGACGACGACAAGGCCCCCAAGAAGAAGAGGAAGGTCGACATGCT
GATCGGCTATGTGCGGGTGTCCACCAACGACCAGAACACCGACCTGCAGA
GAAACGCCCTTGTGTGTGCCGGATGCGAGCAGATCTTCGAGGATAAGCTG
AGCGGCACCAGAACCGACAGACCCGGACTGAAGAGAGCCCTGAAGAGACT
GCAGAAAGGCGACACCCTGGTCGTGTGGAAGCTGGATAGACTGGGCCGCA
GCATGAAGCACCTGATCAGCCTTGTGGGCGAGCTGAGAGAGCGGGGCATC
AACTTTAGAAGCCTGACCGACAGCATCGACACAAGCAGCCCTATGGGCAG
ATTCAACTTCCATGTGATGGGCGCCCTGGCCGAGATGGAAAGAGAGCTGA
TCCTGGAGAGAGTCATGGCCGGAATCGCCGCTGCCAGAAACAAGGGAAGA
AGATGGGGCCGGCCACCTAAGTCTGGCACAGGCGAGAGGCCCTTCCAGTG
TCGAATCTGCATGCGTAACTTCAGTCTGCGCCACCACCTGACCCGCCACA
TCCGCACCCACACCGGCGAGAAGCCTTTTGCCTGTGACATTTGTGGGAGG
AAATTTGCCCAGTCCTACGCCCGCACCCTGCATACCAAGATACACACGCA
TCCCAGGGCACCTATTCCCAAGCCCTTCCAGTGTCGAATCTGCATGCGTA
ACTTCAGTTGGCGCTCCTCCCTGAAGACCCACATCCGCACCCACACCGGC
GAGAAGCCTTTTGCCTGTGACATTTGTGGGAGGAAATTTGCCGACCGCTC
CAACCGCAAGACCCATACCAAGATACACACGCACCCGCGCGCCCCGATCC
CGAAGCCCTTCCAGTGTCGAATCTGCATGCGTAACTTCAGTGACCGCTCC
AACCTGTCCCGCCACATCCGCACCCACACCGGCGAGAAGCCTTTTGCCTG
TGACATTTGTGGGAGGAAATTTGCCCAGTCCGGCAACCTGGCCCGCCATA
CCAAGATACACCTGCGGCAGAAGGACTGATAAC

Also provided herein are vectors comprising polynucleotide sequences encoding the zinc-finger recombinase protein as described herein. Suitable vectors include, but are not limited to, a plasmid, a viral vector, a mini-circle and a linear DNA form. Host cells containing said polynucleotide sequences or vectors are also provided. Any of the foregoing zinc-finger recombinase proteins, polynucleotides encoding the zinc-finger recombinase protein, vectors or cells may be used in the methods disclosed herein.

Another aspect of the present disclosure relates to a pharmaceutical composition comprising the zinc-finger recombinase protein of the disclosure, which comprises a Gin recombinase catalytic domain variant operatively linked to a zinc-finger nucleotide binding domain, wherein the Gin recombinase catalytic domain variant comprises a Phe104Asn amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35; and a pharmaceutically acceptable carrier.

In another aspect, the disclosure relates to a pharmaceutical composition comprising a polynucleotide encoding the zinc-finger recombinase protein of the disclosure, which comprises a Gin recombinase catalytic domain variant operatively linked to a zinc-finger nucleotide binding domain, wherein the Gin recombinase catalytic domain variant comprises a Phe104Asn amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35; and a pharmaceutically acceptable carrier.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

Methods for Modifying the Genome of a Cell

In one aspect, the present disclosure provides methods for modifying the genome of a cell, the method comprising introducing into the cell the Gin recombinase catalytic domain variant of the disclosure zinc-finger recombinase protein of the disclosure or a polynucleotide encoding the Gin recombinase catalytic domain variant or the zinc-finger recombinase protein of the disclosure. The zinc-finger recombinase proteins (ZFR) of the disclosure are chimeric proteins capable of introducing targeted modifications into cells. ZFRs promote site-specific recombination between DNA targets that consist of two zinc-finger binding sites flanking a central 20-bp core sequence recognized by the recombinase catalytic domain. The customization of the zinc-finger nucleotide binding domains allows for the design of ZFRs that have the capacity of recognizing a broad range of defined DNA targets and direct site-specific integration into endogenous genomic loci.

In another aspect, the present disclosure provides a method for integrating an exogenous nucleotide sequence into a target nucleotide sequence in the genome of a cell, the method comprising introducing into a cell the Gin recombinase catalytic domain variant of the disclosure or the zinc-finger recombinase protein of the disclosure.

In another aspect, the present disclosure provides a method for integrating an exogenous nucleotide sequence into a target nucleotide sequence in a gene of a cell, the method comprising introducing into a cell the Gin recombinase catalytic domain variant of the disclosure or the zinc-finger recombinase protein of the disclosure.

In another aspect, the present disclosure provides a method for integrating an exogenous nucleotide sequence into a target nucleotide sequence in the genome of a cell, the method comprising introducing into a cell a polynucleotide encoding the Gin recombinase catalytic domain variant of the disclosure or the zinc-finger recombinase protein of the disclosure.

In another aspect, the present disclosure provides a method for integrating an exogenous nucleotide sequence into a target nucleotide sequence in a gene of a cell, the method comprising introducing into a cell a polynucleotide encoding the Gin recombinase catalytic domain variant of the disclosure or the zinc-finger recombinase protein of the disclosure.

In another aspect, the present disclosure provides a method for excising a target nucleotide sequence from the genome of a cell, the method comprising introducing into the cell the zinc-finger recombinase of the disclosure or the polynucleotide encoding the zinc-finger recombinase of the disclosure.

In another aspect, the present disclosure provides a method for excising a target nucleotide sequence in a chromosome of a cell, the method comprising introducing into the cell the zinc-finger recombinase of the disclosure or the polynucleotide encoding the zinc-finger recombinase of the disclosure.

In some embodiments, the method of excising a target sequence from the genome of the cell comprises introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method for excising a target sequence from the genome of the cell comprises introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the methods disclosed herein further comprise a knock down of NHEJ or inhibition of NHEJ. Exemplary methods for NHEJ knock down or inhibition include but are not limited to, a small molecule inhibitor, a zinc finger protein transcription factor (ZFP-TF), or a peptide inhibitor. In some embodiments, NHEJ is knocked-down by a small molecule. In some embodiments, NHEJ is knocked down by a small molecule inhibitor including KU0060648, VX-984, W7, Chlorpromazine, Vanillin, Nu7026, Nu7441, Mirin, SCR7, AG14361, M9831 and VXc-296. See, e.g., WO2018/013840. In some embodiments, the NHEJ is knocked down by the small molecule inhibitor KU0060648. In some embodiments, NHEJ is knocked down by ZFP-TFs. See, e.g., US20130253040, US20150335708, US20160296605, U.S. Pat. Nos. 9,943,565, 7,939,327, and WO2012068380. In some embodiments, NHEJ is knocked down by peptide inhibitors. In some embodiments, NHEJ is knocked-out by targeting genes including KU70, KU80, DNA-PK, XRCC4, or DNA Ligase IV using zinc finger nucleases (ZFNs), TALEN, or CRISPR/Cas systems.

A zinc-finger recombinase protein of the disclosure binds the target nucleotide sequence as a dimer, interacting through the catalytic domain of the recombinase, where the DNA-binding domains of the two zinc-finger monomers may interact with distinct target nucleotide sequences. A second zinc-finger recombinase protein of the disclosure binds the donor nucleotide sequence as a dimer. A tetramer is formed between the two zinc finger recombinase dimers (the target dimer and the donor dimer), which is the active form of the recombinase capable of integrating the donor sequence into the target nucleotide sequence. In some embodiments, the two dimers comprise a Gin recombinase catalytic domain.

In order for the zinc-finger recombinase protein to catalyze recombination it is necessary for the zinc finger nucleotide binding domain to recognize and bind to an appropriate DNA fragment. Typically, the DNA sequence may comprise two zinc-finger protein binding-sites recognized by the zinc finger nucleotide binding domain of the zinc-finger recombinase protein, flanking a central sequence which interacts with Gin catalytic domain.

The zinc finger nucleotide binding domain is optimized for recognition of their targets. This can be done completely separately from the recombination system, using methods well known to those skilled in the art; mutagenesis followed by ‘phage display’ selection, swapping of parts from known variants of the DNA-binding domain, etc. (Pabo et al., Annu Rev Biochem. 2001; 70:313-40).

The methods and compositions disclosed herein can be used in any type of cell including a eukaryotic or prokaryotic cell and/or cell line. Examples of cells include, but are not limited to, prokaryotic cells, fungal cells, Archaeal cells, plant cells, insect cells, animal cells, vertebrate cells, mammalian cells and human cells. In some embodiments, the cell is a eukaryotic cell. In some embodiments, the cell is a mammalian cell. In some embodiments, the mammalian cell is a stem cell. In some embodiments, the eukaryotic cell is a human cell. In some embodiments, the eukaryotic cell is a plant cell. Non-limiting examples of eukaryotic cells or cell lines generated from such cells include T-cells, COS, K562, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), primary human hepatocyte (PHH), and perC6 cells as well as insect cells such as Spodoptera fugiperda (Sf), or fungal cells such as Saccharomyces, Pichia and Schizosaccharomyces. Examples of stem cells include, but are not limited to, embryonic stem cells, induced pluripotent stem cells (iPS cells), hematopoietic stem cells, neuronal stem cells and mesenchymal stem cells.

In some embodiments, any of the methods for modifying the genome of a cell, the method for integrating an exogenous nucleotide sequence or the method for deleting (excising) a target nucleotide sequence described herein is independent of the Fis enhancer system.

In some embodiments, in order to introduce the zinc finger recombinase protein into the cell, the polynucleotide encoding the zinc-finger recombinase protein is incorporated into a plasmid, a viral vector, a mini-circle or a linear DNA form.

In some embodiments of the methods disclosed herein, the zinc finger nucleotide binding domain comprises the amino acid sequence set forth in SEQ ID NO: 9 or SEQ ID NO: 10.

In some embodiments of the method for integrating an exogenous sequence into a target sequence or for deleting a target nucleotide sequence, the target nucleotide sequence comprises the sequence set forth in SEQ ID NO: 15.

In some embodiments, the target nucleotide sequence is an endogenous locus. In some embodiments, the endogenous locus is selected from the group consisting of Hypoxanthine-guanine phosphoribosyltransferase (HPRT) gene, T Cell Receptor Alpha Constant (TRAC), and a safe-harbor locus. In some embodiments, the endogenous locus is selected from the group consisting of Hypoxanthine-guanine phosphoribosyltransferase (HPRT) gene, T Cell Receptor Alpha Constant (TRAC), Adeno-Associated Virus Integration Site 1 (AAVS1) and a safe-harbor locus.

In some embodiments, the disclosure provides for the integration of an exogenous nucleic acid sequence into a safe harbor locus in the genome of a cell. A safe harbor locus is typically a genomic locus where transgenes can integrate and function in a predictable manner without perturbing endogenous gene activity. Exemplary safe harbor loci in the human genome include, without limitation the Rosa26 locus, the AAVS1 locus, and the safe harbor loci listed in Sadelain et al. Nat Rev Cancer. 2012; 12(1):51-8. In some embodiments, the safe harbor locus is located in chromosome 1.

The mechanism of targeted integration of an exogenous nucleotide sequence into a target nucleotide sequence is the following:
(a) A first zinc finger recombinase forms a dimer by recognizing the target nucleotide sequence;
(b) A second zinc finger recombinase forms a dimer by recognizing the donor nucleotide sequence (the exogenous nucleotide sequence);
(c) A tetramer is formed between the first and second zinc finger recombinase dimers (the target dimer and the donor dimer);
(d) The two recombinase dimers rotate around the tetramer interface, which permits the strand exchange while each recombinase dimer stays covalently linked to the DNA; and
(e) Religation of the nucleotide fragments, which results in strand exchange and introduction of an exogenous nucleotide sequence (the donor nucleotide sequence) into the target sequence.

The mechanism of targeted excision of an endogenous nucleotide sequence is the following:
(a) A first zinc finger recombinase forms a dimer by recognizing a first target nucleotide sequence flanking a first end of a nucleotide sequence to be excised;
(b) A second zinc finger recombinase forms a dimer by recognizing a second target nucleotide sequence flanking a second end of a nucleotide sequence to be excised (the first and second target nucleotide sequences may or may not be identical);
(c) A tetramer is formed between the first and second zinc finger recombinase dimers;
(d) The two recombinase dimers rotate around the tetramer interface, which permits DNA strand exchange while each recombinase dimer stays covalently linked to the DNA;
(e) Excision of the endogenous nucleotide sequence located between the first and second target nucleotide sequence; and
(f) Ligation of the endogenous nucleotide sequences flanking the excised target nucleotide sequence and restoration of chromosomal DNA sequence.

In some embodiments, each zinc-finger recombinase dimer is independently a homodimer. In some embodiment, each zinc-finger recombinase dimer is independently a heterodimer. In some embodiments, the tetrameric zinc finger recombinase is a homotetramer. In other embodiments, the tetrameric zinc-finger recombinase is a heterotetramer.

In some embodiments, each of the two recombinase dimers comprise a gamma Gin recombinase catalytic domain. In some embodiments, one of the two recombinase dimers comprise a gamma Gin recombinase catalytic domain.

The zinc finger recombinase protein or the polynucleotide encoding the zinc finger recombinase protein may be delivered to isolated cells (which in turn may be administered to a living subject for ex vivo cell therapy) or to a living subject. Delivery of gene editing molecules to cells and subjects are known in the art. Methods of delivering zinc finger recombinase proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Suitable cells include, but are not limited to, eukaryotic and prokaryotic cells and/or cell lines. Non-limiting examples of eukaryotic cells or cell lines generated from such cells include T-cells, COS, K562, CHO (e.g., CHO—S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces. In some embodiments, the cell is a mammalian cell. In some embodiments, the cell is a stem cell, such as, by way of example, embryonic stem cells, induced pluripotent stem cells (iPS cells), hematopoietic stem cells, neuronal stem cells and mesenchymal stem cells.

The polynucleotide encoding the zinc finger recombinase protein, as described herein, may also be delivered using vectors containing sequences encoding one or more of the components of the zinc finger recombinase protein. In some embodiments, additional nucleic acids (e.g., donor sequences) also may be delivered via these vectors. Furthermore, it will be apparent that any of these vectors may comprise one or more DNA-binding protein-encoding sequences and/or additional nucleic acids as appropriate. Thus, when one or more zinc finger recombinase protein as described herein are introduced into the cell, and additional DNAs as appropriate, they may be carried on the same vector or on different vectors. When multiple vectors are used, each vector may comprise a sequence encoding one or multiple zinc finger recombinase proteins and additional nucleic acids as desired. Conventional viral and non-viral based gene transfer methods can be used to introduce nucleic acids encoding engineered DNA-binding proteins in cells (e.g., in mammalian cells) and target tissues and to co-introduce additional nucleotide sequences as desired. Such methods can also be used to administer nucleic acids to cells in vitro. In certain embodiments, nucleic acids are administered for in vivo or ex vivo gene therapy uses.

Gene therapy vectors comprising the polynucleotide encoding the zinc finger recombinase protein of the disclosure can be delivered in vivo by administration to an individual patient (subject), typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have incorporated the vector.

Ex vivo cell transfection for diagnostics, research, transplant or for gene therapy (e.g., via re-infusion of the transfected cells into the host organism) is well known to those of skill in the art. In some embodiments, cells are isolated from the subject organism, transfected with a polynucleotide encoding the zinc finger recombinase protein, and re-infused back into the subject organism (e.g., patient). Various cell types suitable for ex vivo transfection are well known to those of skill in the art (see, e.g., Freshney, et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba, et al. (1992) J. Exp. Med. 176:1693-1702).

Methods of treating a disorder in a subject or method for correcting a disease-causing mutation

In one aspect, the disclosure provides a method for treating a disease disorder in a subject, the method comprising modifying a target sequence in the genome of the cell by introducing into the cell the Gin recombinase catalytic domain variant of the disclosure or the zinc-finger recombinase protein of the disclosure.

In another aspect, the disclosure provides a method for treating a disease or disorder in a subject, the method comprising modifying a target sequence in the genome of the cell by introducing into the cell a polynucleotide encoding the Gin recombinase catalytic domain variant of the disclosure or the zinc-finger recombinase protein of the disclosure.

In another aspect, the disclosure provides a method for treating a disorder in a subject, the method comprising excising a target sequence from the genome of the cell by introducing into the cell the zinc-finger recombinase of the disclosure.

In another aspect, the disclosure provides a method for treating a disorder in a subject, the method comprising excising a target sequence from the genome of the cell the polynucleotide encoding a zinc-finger recombinase of the disclosure.

In some embodiments, the method for treating a disorder in a subject, comprises excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method for treating a disorder in a subject, comprises excising a target sequence from the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In another aspect, the disclosure provides a method for correcting a disease- or disorder-causing mutation in the genome of a cell, the method comprising modifying a target sequence in the genome of the cell by introducing into the cell the Gin recombinase catalytic domain variant of the disclosure or the zinc finger recombinase protein of the disclosure.

In another aspect, the disclosure provides a method for correcting a disease- or disorder-causing mutation in the genome of a cell, the method comprising modifying a target sequence in the genome of the cell by introducing into the cell a polynucleotide encoding the Gin recombinase catalytic domain variant of the disclosure or the zinc finger recombinase protein of the disclosure.

In another aspect, the disclosure provides a method for correcting a disease-causing mutation in the genome of a cell, the method comprising excising a target sequence from the genome of the cell by introducing into the cell the zinc-finger recombinase of the disclosure.

In another aspect, the disclosure provides a method for correcting a disease-causing mutation in the genome of a cell, the method comprising excising a target sequence in the genome of the cell by introducing into the cell the polynucleotide encoding a zinc-finger recombinase of the disclosure.

In some embodiments, the method for correcting a disease-causing mutation in the genome of a cell, comprises excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35. In some embodiments, the method for treating a disorder in a subject, comprises excising a target sequence from the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

In some embodiments, the method for treating a disorder in a subject or method for correcting a disease-causing mutation in the genome of a cell disclosed herein further comprise a knock down of NHEJ or inhibition of NHEJ. Exemplary methods for NHEJ knock down or inhibition include but are not limited to, a small molecule inhibitor, a zinc finger protein transcription factor (ZFP-TF), or a peptide inhibitor. In some embodiments, NHEJ is knocked-down by a small molecule. In some embodiments, NHEJ is knocked down by a small molecule inhibitor including KU0060648, VX-984, W7, Chlorpromazine, Vanillin, Nu7026, Nu7441, Mirin, SCR7, AG14361, M9831 and VXc-296. In some embodiments, the NHEJ is knocked down by the small molecule inhibitor KU0060648. In some embodiments, NHEJ is knocked down by ZFP-TFs. In some embodiments, NHEJ is knocked down by peptide inhibitors. In some embodiments, NHEJ is knocked-out by targeting genes including KU70, KU80, DNA-PK, XRCC4, or DNA Ligase IV using zinc finger nucleases (ZFNs), TALEN, or CRISPR/Cas systems.

In one embodiment of the method for treating a disease or disorder or for correcting a disease-causing mutation, at least one cell, cell type or tissue comprise a recombination site that is recognized by a zinc finger nucleotide binding domain. This cell(s) is transformed with a donor nucleic acid construct (a “donor construct”) comprising a second recombination sequence and one or more polynucleotides of interest (typically a therapeutic gene). Into the same cell, a zinc finger recombinase protein of the disclosure or a polynucleotide encoding the zinc finger recombinase protein of the disclosure is introduced. The zinc finger recombinase protein specifically recognizes the recombination sequences forming a tetramer, under conditions such that the nucleic acid sequence of interest is inserted into the genome via a recombination event between the first and second recombination sites. Subjects treatable using the methods of the invention include both humans and non-human animals.

A variety of diseases or disorders may be treated by employing the methods of the disclosure. Non-limiting examples include monogenic disorders, infectious diseases, acquired disorders, cancer, and the like. Exemplary monogenic disorders include ADA deficiency, cystic fibrosis, familial-hypercholesterolemia, hemophilia, chronic granulomatous disease, Duchenne muscular dystrophy, Fanconi anemia, sickle-cell anemia, Gaucher's disease, Hunter syndrome, X-linked SCID, and the like.

Genetic disease or disorders may also be treated or prevented using the methods disclosed herein. Exemplary genetic diseases that may be treated and/or prevented by zinc finger recombinase protein and methods described herein include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangiectasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, phenylketonuria (PKU). porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240), Charcot Marie Tooth (CMT) disease, Autosomal dominant polycystic kidney disease (ADPKD), and the like.

The methods disclosed herein also allow for treatment of infections (viral or bacterial) in a host (e.g., by blocking expression of viral or bacterial receptors, thereby preventing infection and/or spread in a host organism). Non-limiting examples of viruses or viral receptors that may be targeted include herpes simplex virus (HSV), such as HSV-1 and HSV-2, varicella zoster virus (VZV), Epstein-Barr virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses or their receptors may be targeted, including, but not limited to, Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae; lentiviruses (e.g., HTLV-I; HTLV-II; HIV-1 (also known as HTLV-III, LAV, ARV, hTLR, etc.) HIV-II); simian immunodeficiency virus (SIV), human papillomavirus (HPV), influenza virus and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses. Also included are infections with other pathogenic organisms such as Mycobacterium Tuberculosis, Mycoplasma pneumoniae, and the like or parasites such as Plasmodium falciparum, and the like.

The method for treatment or correction of a disease-causing mutation can take place in vivo or ex vivo. By “in vivo” it is meant in the living body of an animal. By “ex vivo” it is meant that cells or organs are modified outside of the body, such cells or organs are typically returned to a living body.

Methods for the therapeutic administration of vectors or constructs including the zinc finger recombinase proteins of the disclosure are well known in the art. Nucleic acid constructs can be delivered with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995, all of which are incorporated by reference herein), using viral vectors (Monahan, et al, Gene Therapy 4:40-49, 1997; Onodera, et al, Blood 91:30-36, 1998, all of which are incorporated by reference herein), by uptake of “naked DNA”, and the like. Techniques well known in the art for the transfection of cells (see discussion above) can be used for the ex vivo administration of nucleic acid constructs. The exact formulation, route of administration and dosage can be chosen by the individual physician in view of the patient's condition. (Fingl et al., 1975, in “The Pharmacological Basis of Therapeutics”, Ch. 1 pl).

As described herein, the zinc finger recombinase protein and methods described herein can be used for genome modification, genome correction, and genome disruption.

The zinc finger recombinase protein and methods described herein can also be applied to stem cell based therapies, including but not limited to editing that results in: correction of somatic cell mutations; disruption of dominant negative alleles; disruption of the genome required for the entry or productive infection of pathogens into cells; enhanced tissue engineering, for example, by editing genome activity to promote the differentiation or formation of functional tissues; and/or disrupting genome activity to promote the differentiation or formation of functional tissues; blocking or inducing differentiation, for example, by editing the genes that block differentiation to promote stem cells to differentiate down a specific lineage pathway. Cell types for this procedure include but are not limited to, T-cells, B cells, hematopoietic stem cells, and embryonic stem cells. Additionally, induced pluripotent stem cells (iPSC) may be used which would also be generated from a patient's own somatic cells. Therefore, these stem cells or their derivatives (differentiated cell types or tissues) could be potentially engrafted into any person regardless of their origin or histocompatibility.

As noted above, the zinc finger recombinase protein and methods described herein can be used for gene modification, gene correction, and gene disruption.

The zinc finger recombinase protein and methods described herein can also be applied to stem cell based therapies, including but not limited to editing that results in: correction of somatic cell mutations; disruption of dominant negative alleles; disruption of genes required for the entry or productive infection of pathogens into cells; enhanced tissue engineering, for example, by editing gene activity to promote the differentiation or formation of functional tissues; and/or disrupting gene activity to promote the differentiation or formation of functional tissues; blocking or inducing differentiation, for example, by editing genes that block differentiation to promote stem cells to differentiate down a specific lineage pathway. Cell types for this procedure include but are not limited to, T-cells, B cells, hematopoietic stem cells, and embryonic stem cells. Additionally, induced pluripotent stem cells (iPSC) may be used which would also be generated from a patient's own somatic cells. Therefore, these stem cells or their derivatives (differentiated cell types or tissues) could be potentially engrafted into any person regardless of their origin or histocompatibility.

In addition to therapeutic applications, the zinc finger recombinase protein and methods described herein can be used for cell line engineering and the construction of disease models.

### EXAMPLES

### Example 1: Zinc Finger Recombinase Protein Design

A Gin recombinase catalytic domain (including (i) H106Y mutation (SEQ ID NO: 2); (ii) F104N mutation (SEQ ID NO: 3) and (iii) no modifications (SEQ ID NO: 1)) was each independently fused to the N-terminus of a zinc finger DNA binding domain using the linker KSGTG (SEQ ID NO: 8). A Gin core sequence scoring algorithm was used to identify Gin core sequences in the HPRT gene, as well as safe harbor site on chromosome 1.

The left (SEQ ID NO: 9) and right (SEQ ID NO: 10) arms of the zinc finger nucleotide binding domain were designed to target a sequence from the HPRT gene.

The zinc finger nucleotide binding domains or ZFPs were designed to target the sequences flanking the Gin core sequences. The Gin recombinase catalytic domain was selected to target the Gin core sequence. For HPRT target site, the Gin core sequence was SEQ ID NO: 12. For the safe harbor site on chromosome 1 target site, the Gin core sequence was SEQ ID NO: 27 (CTAATTACCTTCATTATAGT) and SEQ ID NO: 28 (ACAAATAGTATAATTTGAGT).

Gamma Gin recombinase catalytic domains were codon-optimized for humans, synthesized as gBlocks and cloned into a mammalian expression vector pVax. Gin variants mutations at position H106Y (protein sequence—SEQ ID NO: 2) or F104N (protein sequence—SEQ ID NO: 3) were generated using the NEB® site-directed mutagenesis kit according to the manufacturer's instructions and using the starting codon-optimized Gamma Gin recombinase catalytic domains as a template. Gin variant sequences were confirmed via Sanger sequencing.

Zinc finger DNA binding domains capable of binding HPRT gene were assembled and cloned into the Gin expression vector. Clones sequences were confirmed via Sanger sequencing.

| | Zinc finger DNA binding domain amino acid sequence |
|---|---|
| left arm | SEQ ID NO: 9 |
| right arm | SEQ ID NO: 10 |

The full pVAX plasmid DNA sequence of the left arm of the zinc finger recombinase protein comprising F104N mutation is SEQ ID NO: 16 and the full plasmid DNA sequence of the right arm of the zinc finger recombinase protein comprising F104N mutation is SEQ ID NO: 17.

### Example 2: Donor Construct Design

Site specific donor molecules were cloned by inserting the zinc finger recombinase sites (ZFP binding site: Gin core sequence: ZFP binding site) into a pUC19 vector backbone. The donor construct was generated using HiFi Assembly kit (NEB®). The donor fragment was synthesized by IDT® and provided as gene fragments (gBlock), including HiFi assembly overhangs. The donor construct comprised the binding sites of the two zinc finger nucleotide binding domains flanking the Gin core sequence (ZFP binding site-Gin core sequence-ZFP binding site).

The pUC19 vector backbone was PCR amplified. The gBlock and primer sequences are shown below.

gBlock nucleotide sequence.
SEQ ID NO: 20
taaaacgacggccagtgaattcATGAAAAGGTGGAAGCTTTACTTGGTTA
TAACTTTACTTTTAATACCTAGAACAGTGAGTCTTCAAACTGCTTTATTG
ATGCCCAATTTATAAAAAGTTTCCTGAGCATTTACCCCTAATATATGCtc
tgctaatggagctatgcaat
pUC19 forward primer used to clone the gBlock
fragment into pUC19.
SEQ ID NO: 21
tgctaatggagctatgcaatCTACTGAATGTCATAATCAACTTC
pUC19 reverse primer used to clone the gBlock
fragment into pUC19.
SEQ ID NO: 22
attcactggccgtcgtfttaCAACGTCGTGACTGGGAAAAC

Additional sequence information (including one of the genomic primer binding sites) was added to support a Standard Next Generation sequencing (NGS)-based Target Integration (TI) assay. A unique tag sequence was added to differentiate between wildtype zinc finger recombinase protein (SEQ ID NO: 23—TGTATTTGC) and target-integrated (TI) PCR amplicons (SEQ ID NO: 24—GCTTTATTG). Recombinase-mediated TI events resulted in reads with the TI tag.

### Example 3: Targeted Integration in Human K562 Cells

K562 (ATCC® CCL243™) cells were obtained from ATCC and were maintained in RPMI1640 medium with 10% FBS and 1×PSG at 37° C. with 5% CO₂ atmosphere.

Various doses of plasmid DNA encoding the zinc finger recombinase protein (H106Y variant or F104N variant) with or without the donor molecule (also supplied as pDNA) were electroporated into K562 cells using the SF cell line 96-well Nucleofector™ kit (Lonza®, V4SC-2960) following the manufacturer's instructions.

In brief, cells were washed once with 1×PBS (divalent cation-free) and resuspended at 2×10⁵ cells per 13 μl of supplemented SF cell line 96-well Nucleofector™ solution. For each transfection, 13 μl of the cell suspension was mixed with 7 μl of pDNA and transferred to the Lonza® Nucleocuvette™ plate and electroporated using the protocol for K562 cells (Nucleofector™ program 96-FF-120) on an Amaxa Nucleofector™ 96-well Shuttle System)(Lonza®). Electroporated cells were incubated at room temperature for 10 min and then transferred to 180 μl of prewarmed complete medium in a 96-well tissue culture plate. Cells were incubated for 72 h and then harvested for targeted integration quantification. Genomic DNA was extracted using QuickExtract™ following the manufacturer's instructions. Cells were subjected to an optional cold shock at 30° C. overnight before transferring them to 37° C.

### Example 4: Standard NGS-Based Targeted Integration Assay

PCR primers for loci of interest were designed using Primer3 design tool with the following optimal conditions: amplicon size of 200-nt; a melting temperature of 60° C.; primer lengths of 20-nt; and GC content of 50%. Adaptors were added for a second PCR reaction to add the Illumina library sequences (forward primer: ACACGACGCTCTTCCGATCT (SEQ ID NO: 25); reverse primer: GACGTGTGCTCTTCCGAT (SEQ ID NO: 26)). The HPRT locus specific primer sequences used were: SEQ ID NO: 29 (taaaacgacggccagtgaattc) and SEQ ID NO: 30 (tctgctaatggagctatgcaat).

Regions of interest were amplified in 25 μl using 2.5 μl of genomic DNA from the QuickExtract™ gDNA solution with AccuPrime HiFi Polymerase (Invitrogen). Primers were used at a final concentration of 0.1 μM with the following thermocycling conditions: initial melt of 95° C. for 5 min; 30 cycles of 95° C. for 30 s, 55° C. for 30 s and 68° C. for 40×; and a final extension at 68° C. for 10 min.

One microliter of this PCR product was used in a 20 μl PCR reaction to add the Illumina library sequences with Phusion™ High-Fidelity PCR MasterMix with HF Buffer (NEB®). Primers were used at a final concentration of 0.5 μM with the following thermocycling conditions: initial melt of 98° C. for 30 s, 12 cycles for 98° C. for 10 s, 60° C. for 30 s and 72° C. for 40 s; and a final extension at 72° C. for 10 min.

PCR libraries were purified using the QIAquick™ PCR purification kit (Qiagen). Samples were quantified with the Qubit™ dsDNA HS Assay kit (Invitrogen) and diluted to 2 nM.

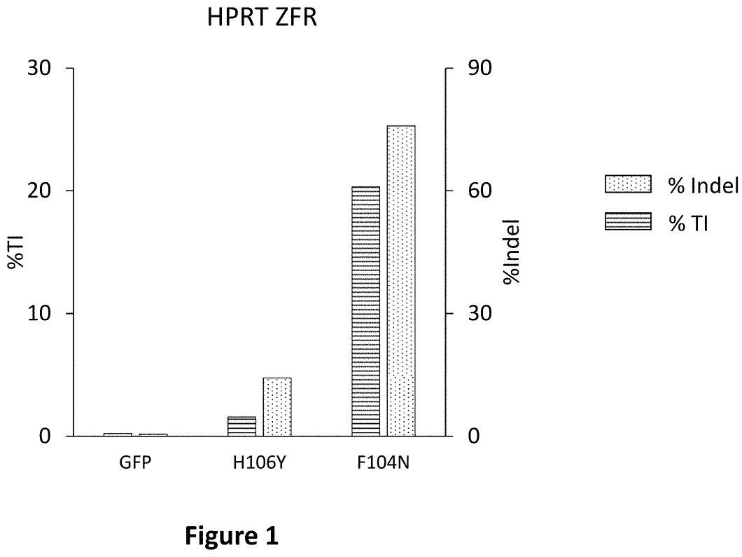

The libraries were then run according to the manufacturer's instructions on either an Illumina® MiSeg™ using a standard 300-cycle kit or an Illumina® NextSeq 500 using a mid-output 300-cycle kit.TI efficiency was determined by quantifying the total number of wild-type reads and the total number of TI reads (including the unique TI sequence tag). See FIG. 1, which shows a graph comparing the target integration efficiency of H106Y zinc finger recombinase variant and F104N zinc finger recombinase variant. The target integration efficiency of the F104N zinc finger recombinase variant is around 20%, while the target integration efficiency of the H106Y zinc finger recombinase variant is around 1.5%.

### Example 5: Flow Cytometry Assay

A GFP expression cassette was cloned into the donor vector backbone described above. Transfections were performed as described and cells were maintained for 3 weeks. After 3 weeks, unincorporated donor molecules had been washed out and only stably integrated donor molecules resulted in GFP expression.

1×10⁶K562 cells were washed in PBS, resuspended in 200 μl FACS buffer (Ca2+/Mg2+ free PBS, 1% BSA) and subjected to GFP quantification via flow cytometry assay, where the transfection efficiency was determined.

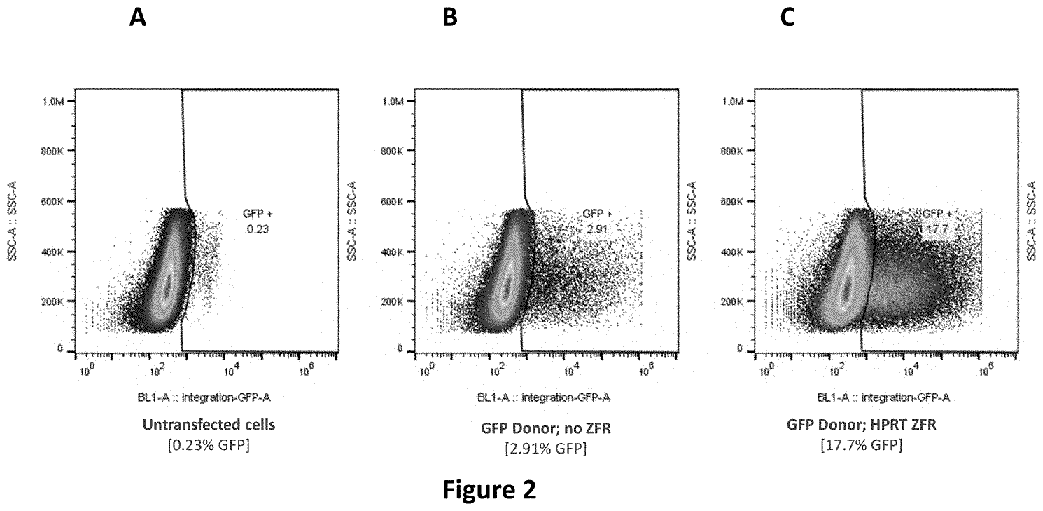

Flow cytometry was performed on an Invitrogen® Attune NxT flow cytometer following the manufacturer's instructions. The percentage of GFP expressing cells was compared between non-transfected cells showing background signal (FIG. 2, Panel A), cells transfected only with the donor molecule showing random GFP integration (FIG. 2, Panel B) and cells transfected with the F104N Zinc Finger recombinase variant together with the donor construct (FIG. 2, Panel C). As shown in FIG. 2, the GFP fluorescence detected in cells transfected with F104N Zinc Finger recombinase variant and HPRT donor construct is much higher than the fluorescence detected in cells transfected with the donor only (17.7% vs 2.91%).

### Example 6: Zinc Finger Nuclease Design

Zinc finger proteins targeted to HPRT were designed and incorporated into plasmids as described elsewhere. Table A shows the recognition helices within the DNA binding domains of two exemplary HPRT ZFNs (N>C finger sequence). Table B shows the target sites for these ZFNs (DNA target sites indicated in uppercase letters; non-contacted nucleotides indicated in lowercase).

TABLE A
HPRT-specific zinc finger nuclease helix design

| | F1 | F2 | F3 | F4 | F5 | F6 |
|---|---|---|---|---|---|---|
| SB85969 | LRHHLTR | DRSTLRQ | QSANRTK | DNSYLPR | RSDNLSV | QKATRIN |
| | (SEQ ID | (SEQ ID | (SEQ ID | (SEQ ID | (SEQ ID | (SEQ ID |
| | NO: 66) | NO: 67) | NO: 68) | NO: 69) | NO: 70) | NO: 71) |
| SB85973 | RSDNLAR | LRQHLRA | QSGNLAR | QSTPRTT | DRSNRIK | |
| | (SEQ ID | (SEQ ID | (SEQ ID | (SEQ ID | (SEQ | ID |
| | NO: 72) | NO: 73) | NO: 74) | NO: 75) | NO: 76) | |

TABLE B
Target site of HPRT-specific zinc finger nucleases

| | |
|---|---|
| SB85969 | 5′-3′: GTAAAGTTATAACCAAGT (SEQ ID NO: 77) |
| SB85973 | 5′-3′: TACCTAGAAcAGTGAG (SEQ ID NO: 78) |

### Example 7: ZFR-Mediated Target Integration (TI) Versus ZFN-Mediated TI

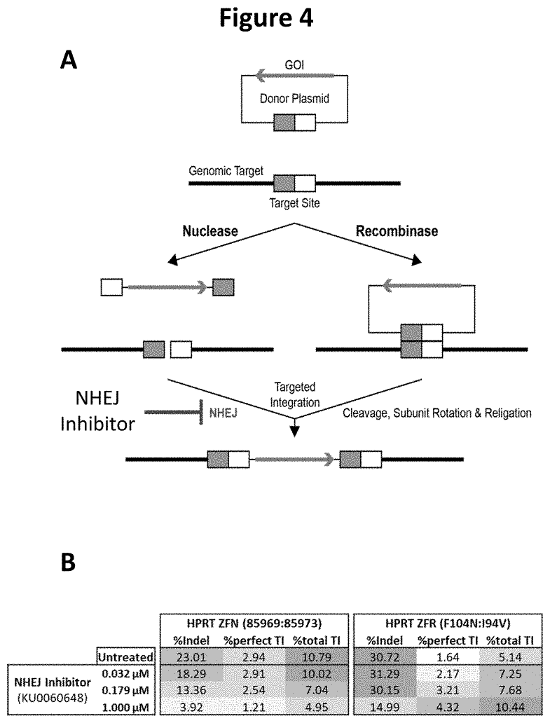

HPRT ZFN control (see Example 6) or HPRT ZFR constructs (F104N:I94V) were co-transfected with donor plasmids into K562 cells as described in Example 3. KU0060648, a NHEJ inhibitor at a concentration of 0 μM, 0.032 μM, 0.179 μM or 1.00 μM was added to the recovery media. Cells were harvested after 3 days for subsequent PCR-based NGS analysis as described in Example 4. As shown in FIG. 4, Panel B, ZFN-induced NHEJ-mediated targeted integration in the presence of KU0060648 was negatively impacted. % Indel, % perfect TI (Indel-free TI) and % total TI all decreased as the dose of the NHEJ inhibitor increased. In contrast, the ZFR (F104N:I94V) showed an increase in both % perfect TI and % total TI increased while % Indel dropped as KU0060648 dose increased (FIG. 4, Panel B). This demonstrates that the ZFR-mediated targeted integration mechanism is distinct from ZFN-induced NHEJ-mediated targeted integration.

### Example 8: Preparation of a Stable Excision Reporter Cell Line

A K562 reporter cell line with two HPRT ZFR binding sites was generated. First, an excision reporter construct was cloned into a plasmid. The sequence of the plasmid is set forth in SEQ ID NO: 81. The reporter construct has two binding sites for the HPRT ZFR, spaced 2888-nt apart. The HPRT ZFR binding site is as follows:

(SEQ ID NO: 15)
AAGGTGGAAGCTTTACTTGGTTATAACTTTACTTTTAATACCTAGAACA
GTGAGTCTTC.

The cloned excision construct was co-transfected with a ZFN:30035 and 30054 (which cuts both the genome and the plasmid) into K562 cells for integration into the AAVS1 safe locus site by ZFN-induced NHEJ-mediated targeted integration as previously described (see, e.g., Miller, et al., Enhancing gene editing specificity by attenuating DNA cleavage kinetics, Nature Biotechnology (vol. 37, 2019)). The reporter construct comprises a polynucleotide sequence that helps facilitate the integration of the reporter construct into the AAVS1 (see Table C).

A PCR-based assay was used to detect integration of the HPRT donor construct into the AAVS1 locus as described in Example 4. The primer binding site targeted to detect proper integration into AAVS1 is set forth in Table C. A clonal K562 reporter cell line was selected after proper detection of HPRT donor integration into the AAVS1 locus.

TABLE C
SEQ ID NO: 79
GCCCCACTGTGGGGTGGAGGGGA [symbol] [symbol] [symbol]
CAAACCGGCCCTGGGAATATAAGGCTCAGACCATCCTTCCAGACTCA
GGAGATCATGTGTTGCCGACCGTCCTTCCGTCAAGCGGGTTTTAGAGATTC
AAGGTCGAGAAGCAAAAGCAGCAGGAGATGCTTCTCTGCATCCGTCTTAA
ACTGCTTTGAGACATGAAAAGGTGGAAGCTTTACTTGGTTATAACTTTACTT
TTAATACCTAGAACAGTGAGTCTTCAAACTGCTTTATTGATGCCCAATTTAT
AAAAAGTTTCCTGAGGCGGACTGGACGTAAAACATTCTGCTAATGGAGCTA
TGCAATCTACTGAATGTCATAATCAACTTCAAGGATATACTGAAGCTATGT
GGGCTTCTCATGAAAGAGAATTTTTTGCTTGTAAGGAAAGATGTATGCTTG
AAATAAGCCACCATAGAGACCCATGTCCTAACAGCTCCCTGGAGACGGCTA
ATGATACTTGGAAGCTTGGCGTAATCATGGTCATAGCTGTTTCCTGTGTGA
AATTGTTATCCGCTCACAATTCCACACAACATACGAGCCGGAAGCATAAAG
TGTAAAGCCTGGGGTGCCTAATGAGTGAGCTAACTCACATTAATTGCGTTG
CGCTCACTGCCCGCTTTCCAGTCGGGAAACCTGTCGTGCCAGCTGCATTAA
TGAATCGGCCAACGCGCGGGGAGAGGCGGTTTGCGTATTGGGCGCTCTTCC
GCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGAT
AACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGAC
TTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACT
AGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAAC
TCACGTTAAGGGATTTTGGTCATGAGATTATCAAAAAGGATCTTCACCTAG
ATCCTTTTAAATTAAAAATGAAGTTTTAAATCAATCTAAAGTATATATGAG
TAAACTTGGTCTGACAGTTACCAATGCTTAATCAGTGAGGCACCTATCTCA
GCGATCTGTCTATTTCGTTCATCCATAGTTGCCTGACTCCCCGTCGTGTAGA
TAACTACGATACGGGAGGGCTTACCATCTGGCCCCAGTGCTGCAATGATAC
CGCGAGACCCACGCTCACCGGCTCCAGATTTATCAGCAATAAACCAGCCAG
CCGGAAGGGCCGAGCGCAGAAGTGGTCCTGCAACTTTATCCGCCTCCATCC
AGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTC
GTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGAT
CGTTGTCAGAAGTAAGTTGGCCGCAGTGTTATCACTCATGGTTATGGCAGC
ACTGCATAATTCTCTTACTGTCATGCCATCCGTAAGATGCTTTTCTGTGACT
GGTGAGTACTCAACCAAGTCATTCTGAGAATAGTGTATGCGGCGACCGAGT
TGCTCTTGCCCGGCGTCAATACGGGATAATACCGCGCCACATAGCAGAACT
TTAAAAGTGCTCATCATTGGAAAACGTTCTTCGGGGCGAAAACTCTCAAGG
ATCTTACCGCTGTTGAGATCCAGTTCGATGTAACCCACTCGTGCACCCAACT
GATCTTCAGCATCTTTTACTTTCACCAGCGTTTCTGGGTGAGCAAAAACAG
GAAGGCAAAATGCCGCAAAAAAGGGAATAAGGGCGACACGGAAATGTTG
AATACTCATACTCTTCCTTTTTCAATATTATTGAAGCATTTATCAGGGTTAT
TGTCTCATGAGCGGATACATATTTGAATGTATTTAGAAAAATAAACAAATA
GGGGTTCCGCGCACATTTCCCCGAAAAGTGCCACCTGACGTCTAAGAAACC
ATTATTATCATGACATTAACCTATAAAAATAGGCGTATCACGAGGCCCTTT
CGTCTCGCGCGTTTCGGTGATGACGGTGAAAACCTCTGACACATGCAGCTC
CCGGAGACGGTCACAGCTTGTCTGTAAGCGGATGCCGGGAGCAGACAAGC
CCGTCAGGGCGCGTCAGCGGGTGTTGGCGGGTGTCGGGGCTGGCTTAACTA
TGCGGCATCAGAGCAGATTGTACTGAGAGTGCACCATATGCGGTGTGAAAT
ACCGCACAGATGCGTAAGGAGAAAATACCGCATCAGGCGCCATTCGCCAT
TCAGGCTGCGCAACTGTTGGGAAGGGCGATCGGTGCGGGCCTCTTCGCTAT
TACGCCAGCTGGCGAAAGGGGGATGTGCTGCAAGGCGATTAAGTTGGGTA
ACGCCAGGGTTTTCCCAGTCACGACGTTGTAAAACGACGGCCAGTGAATTC
ATGAAAAGGTGGAAGCTTTACTTGGTTATAACTTTACTTTTAATACCTAGA
ACAGTGAGTCTTCAAACTTGTATTTGCATGCCCAATTTATAAAAAGTTTCCT
GAGGCGGACTGGACGTAAAACATGAGCAACGACCACAACACACCCACAGG
GACAAACACCGCCCAAGTCAAAGATCACTCATGTCGTCGAGGAAGTGGAG
GTTGCTTACATGGCTCCTTCTCTGGACAGTGTGTCACCAGATAAGGAATC
TGCCTAACAGGAGGTGGG [symbol] AATATCAGGAGACTAGGAAG

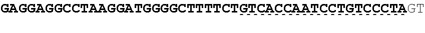

G
Legend:
Bold: Sequence tags for successful integration of the reporter construct into the AAVS1 locus
Underlined: 30035 binding site [symbol]
: 30054 binding site
Italics: Target integration tag
Double underlined: PCR assay: primer binding site

### Example 9: ZFR (H106Y)-Mediated Targeted Excision is Enhanced with NHEJ Inhibition

Targeted excision was induced following transfection of the clonal K562 cell line with a H106Y ZFR, F104N ZFR or ZFN construct. The cells were treated with 0 μM or 3.14 μM KU0060648, a NHEJ inhibitor post-transfection. A PCR-based NGS assay was performed as described in Example 4. The following primers were used to detect excision events:

Forward primer:
(SEQ ID NO: 80)
ACACGACGCTCTTCCGATCTNNNNATGCTTCTCTGCATCCGTCT
and
the reverse primer:
(SEQ ID NO: 81)
GACGTGTGCTCTTCCGATCTATGTTTTACGTCCAGTCCGC.

Additional sequence information (including one of the genomic primer binding sites) was added to support a Standard Next Generation sequencing (NGS)-based Target Excision assay. A unique tag sequence was added to differentiate between a lack of ZFR excision activity (SEQ ID NO: 24—GCTTTATTG) and detectable targeted excision (SEQ ID NO: 23—TGTATTTGC) induced by the transfected ZFR constructs.

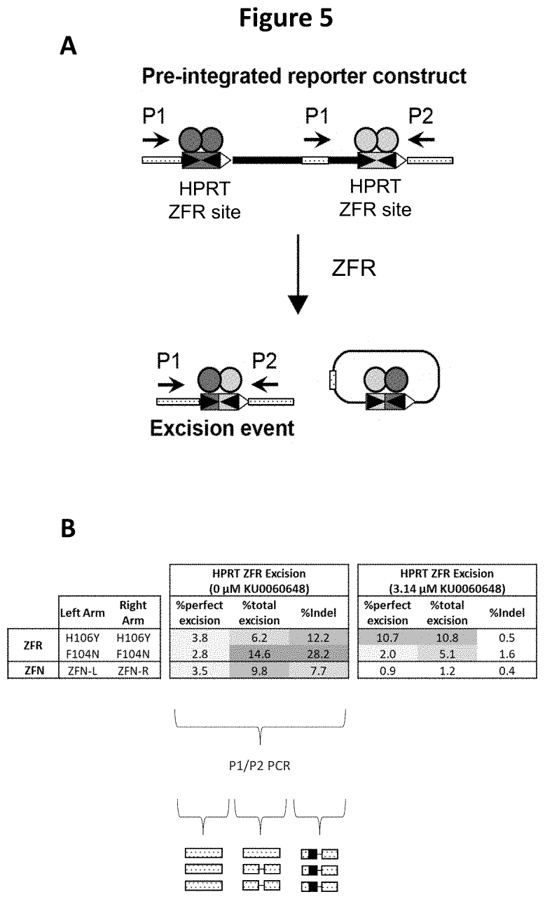

As shown in FIG. 5, Panel B, in the absence of a NHEJ inhibitor (KU0060648), ZFN and ZFR variants (H106Y and F104N) targeting the ZFR binding site efficiently excised the DNA molecule flanked by the two ZFR sites as measured by % perfect excision (Indel-free target locus after excision), % total excision (non-perfect events), and % Indels within the non-excised target sites. In the presence of 3.14 μM KU0060648, the ZFNs and F104N ZFR variant had reduced excision events, while the H106Y ZFR variant showed increased % perfect excision and % total excision with decreased % Indel. Although the hyperactive H106Y ZFR variant performed poorly in targeted integration experiments as represented in FIG. 1, the outcome changed in the donor-free application of targeted excision. Therefore, inhibition of NHEJ can be beneficial for both ZFR-mediated targeted integration and ZFR-mediated targeted excision.

### NUMBERED EMBODIMENTS

Particular embodiments of the disclosure are set forth in the following numbered paragraphs:

1. A Gin recombinase catalytic domain variant comprising a Phe104Asn amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

2. The Gin recombinase catalytic domain variant according to paragraph 1, further comprising a His106Tyr amino acid substitution.

3. The Gin recombinase catalytic domain variant according to paragraph 2, comprising the amino acid sequence set forth in any one of SEQ ID NO: 36, SEQ ID NO: 37, SEQ ID NO: 38, SEQ ID NO: 39, SEQ ID NO: 56, or SEQ ID NO: 60.

4. The Gin recombinase catalytic domain variant according to paragraph 1, further comprising an Ile94Val amino acid substitution.

5. The Gin recombinase catalytic domain variant according to paragraph 4, comprising the amino acid sequence set forth in any one of SEQ ID NO: 42, SEQ ID NO: 44, SEQ ID NO: 48, SEQ ID NO: 52, SEQ ID NO: 57, or SEQ ID NO: 61.

6. A polynucleotide encoding a Gin recombinase catalytic domain variant, wherein the nucleic acid sequence encoding the Gin recombinase catalytic domain variant comprises the nucleotide sequence set forth in SEQ ID NO: 7.

7. A polynucleotide encoding a Gin recombinase catalytic domain variant according to any one of paragraphs 1-5.

8. A zinc-finger recombinase, comprising a Gin recombinase catalytic domain variant operatively linked to a zinc-finger nucleotide binding domain, wherein the Gin recombinase catalytic domain variant comprises a Phe104Asn amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

9. The zinc finger recombinase according to paragraph 8, wherein the Gin recombinase catalytic domain variant further comprises a His106Tyr amino acid substitution.

10. The zinc finger recombinase according to paragraph 9, wherein the Gin recombinase catalytic domain variant comprises the amino acid sequence set forth in any one of SEQ ID NO: 36, SEQ ID NO: 37, SEQ ID NO: 38, SEQ ID NO: 39, SEQ ID NO: 56, or SEQ ID NO: 60.

11. The zinc finger recombinase according to paragraph 8, wherein the Gin recombinase catalytic domain variant further comprises an Ile94Val amino acid substitution.

12. The zinc finger recombinase according to paragraph 11, wherein the Gin recombinase catalytic domain variant comprises the amino acid sequence set forth in any one of SEQ ID NO: 42, SEQ ID NO: 44, SEQ ID NO: 48, SEQ ID NO: 52, SEQ ID NO: 57, or SEQ ID NO: 61.

13. The zinc-finger recombinase according to any one of paragraphs 8-12, wherein the zinc-finger recombinase protein is a multimeric protein.

14. The zinc-finger recombinase according to paragraph 13, wherein the zinc-finger recombinase protein is a homomultimeric protein.

15. The zinc-finger recombinase according to paragraph 13, wherein the zinc-finger recombinase protein is a heteromultimeric protein.

16. The zinc-finger recombinase according to paragraph 13, wherein said zinc-finger recombinase protein is a dimeric protein.

17. The zinc-finger recombinase according to paragraph 16, wherein said zinc-finger recombinase protein is a homodimeric protein.

18. The zinc-finger recombinase according to paragraph 16, wherein said zinc-finger recombinase protein is a heterodimeric protein.

19. The zinc-finger recombinase according to paragraph 14, wherein said zinc-finger recombinase protein is a tetrameric protein.

20. The zinc-finger recombinase according to paragraph 19, wherein said zinc-finger recombinase protein is a homotetrameric protein.

21. The zinc-finger recombinase according to paragraph 19, wherein said zinc-finger recombinase protein is a heterotetrameric protein.

22. The zinc-finger recombinase according to any one of paragraphs 8 to 21, wherein the zinc finger nucleotide binding domain comprises the sequence as set forth in SEQ ID NO: 9 or SEQ ID NO: 10.

23. The zinc-finger recombinase according to any one of paragraphs 8 to 22, wherein the zinc finger recombinase protein binds a nucleotide sequence comprising the sequence as set forth in SEQ ID NO: 15.

24. The zinc-finger recombinase according to any one of paragraphs 8 to 23, wherein the zinc finger nucleotide binding domain is capable of binding an endogenous locus.

25. The zinc-finger recombinase according to paragraph 24, wherein the endogenous locus is selected from the group consisting of Hypoxanthine-guanine phosphoribosyltransferase (HPRT) gene, T Cell Receptor Alpha Constant (TRAC) gene, Adeno-Associated Virus Integration Site 1 (AAVS1) and a safe-harbor locus.

26. A polynucleotide encoding the zinc-finger recombinase according to any one of paragraphs 8-25.

27. The polynucleotide according to paragraph 26, wherein the nucleic acid sequence encoding the Gin recombinase catalytic domain variant comprises the nucleotide sequence set forth in SEQ ID NO: 7.

28. A vector comprising the polynucleotide encoding the Gin recombinase catalytic domain variant according to paragraphs 6-7.

29. A vector comprising the polynucleotide encoding a zinc-finger recombinase according to paragraph 26 or 27.

30. A cell comprising the vector according to paragraph 28 or 29.

31. A cell comprising the Gin recombinase catalytic domain variant according to any one of paragraphs 1-5.

32. A cell comprising the polynucleotide encoding the Gin recombinase catalytic domain variant according to paragraphs 6-7.

33. A cell comprising the zinc finger recombinase protein according to any of paragraphs 8-25.

34. A cell comprising the polynucleotide encoding the zinc-finger recombinase according to paragraphs 26-27.

35. The cell according to any one of paragraphs 30 to 34, wherein the cell is a eukaryotic cell.

36. The cell according to paragraph 35, wherein the cell is a mammalian cell.

37. The cell according to paragraph 36, wherein the wherein the cell is a stem cell.

38. The cell according to paragraph 35, wherein the cell is a human cell.

39. A pharmaceutical composition comprising the Gin recombinase catalytic domain variant according to any one of paragraphs 1-5; and a pharmaceutically acceptable carrier.

40. A pharmaceutical composition comprising the polynucleotide encoding the Gin recombinase catalytic domain variant according to paragraphs 6-7; and a pharmaceutically acceptable carrier.

41. A pharmaceutical composition comprising the zinc-finger recombinase according to any one of paragraphs 8 to 25; and a pharmaceutically acceptable carrier.

42. A pharmaceutical composition comprising the polynucleotide encoding a zinc-finger recombinase according to paragraph 26 or 27; and a pharmaceutically acceptable carrier.

43. A method for modifying the genome of a cell, the method comprising introducing into a cell the zinc-finger recombinase according to any one of paragraphs 8-25.

44. A method for modifying the genome of a cell, the method comprising introducing into the cell the polynucleotide encoding a zinc-finger recombinase according to paragraph 26 or 27.

45. A method for integrating an exogenous nucleotide sequence into a target nucleotide sequence in genome of a cell, the method comprising introducing into a cell the zinc-finger recombinase according to any one of paragraphs 8-25.

46. A method for integrating an exogenous nucleotide sequence into a target nucleotide sequence in the genome of a cell, the method comprising introducing into the cell the polynucleotide encoding a zinc-finger recombinase according to paragraph 26 or 27.

47. A method for disrupting a target nucleotide sequence in the genome of a cell, the method comprising introducing into the cell the zinc-finger recombinase according to any one of paragraphs 8-25.

48. A method for disrupting a target nucleotide sequence in the genome of a cell, the method comprising introducing into the cell the polynucleotide encoding a zinc-finger recombinase according to paragraph 26 or 27.

49. A method for excising a target nucleotide sequence from the genome of a cell, the method comprising introducing into the cell the zinc-finger recombinase according to any one of paragraphs 8-25.

50. A method for excising a target nucleotide sequence from the genome of a cell, the method comprising introducing into the cell the polynucleotide encoding a zinc-finger recombinase according to paragraph 26 or 27.

51. A method for excising a target nucleotide sequence from the genome of a cell, the method comprising introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

52. A method for excising a target nucleotide sequence from the genome of a cell, the method comprising introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35.

53. The method according to any one of paragraphs 49-52, further comprising introducing into the cell a non-homologous end joining (NHEJ) inhibitor.

54. The method according to paragraph 53, wherein the NHEJ inhibitor is selected from the group consisting of a small molecule inhibitor, a zinc-finger protein transcription factor (ZFP-TF), and a peptide inhibitor.

55. The method according to paragraph 54, wherein the small molecule inhibitor is selected from the group consisting of KU0060648, VX-984, W7, Chlorpromazine, Vanillin, Nu7026, Nu7441, Mirin, SCR7, AG14361, M9831 and VXc-296.

56. A method for treating a disorder in a subject, the method comprising modifying a target sequence in the genome of the cell by introducing into the cell the zinc-finger recombinase according to any one of paragraphs 8-25.

57. A method for treating a disorder in a subject, the method comprising modifying a target sequence in the genome of the cell by introducing into the cell the polynucleotide encoding a zinc-finger recombinase according to paragraph 26 or 27.

58. A method for treating a disorder in a subject, the method comprising excising a target sequence from the genome of the cell by introducing into the cell the zinc-finger recombinase according to any one of paragraphs 8-25.

59. A method for treating a disorder in a subject, the method comprising excising a target sequence from the genome of the cell the polynucleotide encoding a zinc-finger recombinase according to paragraph 26 or 27.

60. A method for treating a disorder in a subject, the method comprising excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

61. A method for treating a disorder in a subject, the method comprising excising a target sequence from the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

62. The method according to any one of paragraphs 58-61, further comprising administering a non-homologous end joining (NHEJ) inhibitor.

63. The method according to paragraph 62, wherein the NHEJ inhibitor is selected from the group consisting of a small molecule inhibitor, a zinc-finger protein transcription factor (ZFP-TF), and a peptide inhibitor.

64. The method according to paragraph 63, wherein the small molecule inhibitor is selected from the group consisting of KU0060648, VX-984, W7, Chlorpromazine, Vanillin, Nu7026, Nu7441, Mirin, SCR7, AG14361, M9831 and VXc-296.

65. A method for correcting a disease-causing mutation in the genome of a cell, the method comprising modifying a target sequence in the genome of the cell comprising introducing into the cell the zinc-finger recombinase according to any one of paragraphs 8-25.

66. A method for correcting a disease-causing mutation in the genome of a cell, the method comprising modifying a target sequence in the genome of the cell comprising introducing into the cell the polynucleotide encoding a zinc-finger recombinase according to paragraph 26 or 27.

67. A method for correcting a disease-causing mutation in the genome of a cell, the method comprising excising a target sequence from the genome of the cell by introducing into the cell the zinc-finger recombinase according to any one of paragraphs 8-25.

68. A method for correcting a disease-causing mutation in the genome of a cell, the method comprising excising a target sequence in the genome of the cell by introducing into the cell the polynucleotide encoding a zinc-finger recombinase according to paragraph 26 or 27.

69. A method for correcting a disease-causing mutation in the genome of a cell, the method comprising excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

70. A method for correcting a disease-causing mutation in the genome of a cell, the method comprising excising a target sequence in the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

71. The method according to any one of paragraphs 67-70, wherein the method further comprises administering a non-homologous end joining (NHEJ) inhibitor.

72. The method according to paragraph 71, wherein the NHEJ inhibitor is selected from the group consisting of a small molecule inhibitor, a zinc-finger protein transcription factor (ZFP-TF), and a peptide inhibitor.

73. The method according to paragraph 72, wherein the small molecule inhibitor is selected from a group consisting of KU0060648, VX-984, W7, Chlorpromazine, Vanillin, Nu7026, Nu7441, Mirin, SCR7, AG14361, M9831 and VXc-296.

74. The method according to any one of paragraphs 43 to 73, wherein the cell is a eukaryotic cell.

75. The method according to paragraph 74, wherein the cell is a mammalian cell.

76. The method according to paragraph 75, wherein the cell is a stem cell.

77. The method according to paragraph 74, wherein the cell is a human cell.

78. The method according to any one of paragraphs 43 to 73, wherein the method is independent of Fis.

79. The method according to any one of paragraphs 43 to 78, wherein the polynucleotide encoding the zinc-finger recombinase is introduced into the cell using a plasmid, a viral vector, a mini-circle or a linear DNA form.

80. The method according to any one of paragraphs 44 to 78, wherein the target nucleotide sequence is an endogenous locus.

81. The method according to paragraph 80, wherein the endogenous locus is selected from the group consisting of Hypoxanthine-guanine phosphoribosyltransferase (HPRT) gene, T Cell Receptor Alpha Constant (TRAC), Adeno-Associated Virus Integration Site 1 (AAVS1) and a safe-harbor locus.

82. The zinc-finger recombinase according to any one of paragraphs 8-25 for use in modifying the genome of a cell.

83. The polynucleotide encoding a zinc-finger recombinase according to paragraph 26 or 27 for use in modifying the genome of a cell.

84. The zinc-finger recombinase according to any one of paragraphs 8-25 for use in integrating an exogenous nucleotide sequence into a target nucleotide sequence in the genome of a cell.

85. The polynucleotide encoding a zinc-finger recombinase according to paragraph 26 or 27 for use in integrating an exogenous nucleotide sequence into a target nucleotide sequence in the genome of a cell.

86. The zinc-finger recombinase according to any one of paragraphs 8-25 for use in disrupting a target nucleotide sequence in the genome of a cell.

87. The polynucleotide encoding a zinc-finger recombinase according to paragraph 26 or 27 for use in disrupting a target nucleotide sequence in the genome of a cell.

88. The zinc-finger recombinase according to any one of paragraphs 8-25 for use in excising a target nucleotide sequence from the genome of a cell.

89. The polynucleotide encoding a zinc-finger recombinase according to paragraph 26 or 27 for use in excising a target nucleotide sequence from the genome of a cell.

90. A zinc finger recombinase for use in excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

91. A polynucleotide encoding a zinc-finger recombinase for use in excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35.

92. The zinc finger recombinase for use according to paragraph 88 or 90 or polynucleotide encoding a zinc finger recombinase according to paragraph 89 or 91, further comprising the use of a non-homologous end joining (NHEJ) inhibitor.

93. The zinc finger recombinase or polynucleotide encoding a zinc finger recombinase for use according to paragraph 92, wherein the NHEJ inhibitor is selected from the group consisting of a small molecule inhibitor, a zinc-finger protein transcription factor (ZFP-TF), and a peptide inhibitor.

94. The zinc finger recombinase or polynucleotide encoding a zinc finger recombinase for use according to paragraph 93, wherein the small molecule inhibitor is selected from the group consisting of KU0060648, VX-984, W7, Chlorpromazine, Vanillin, Nu7026, Nu7441, Mirin, SCR7, AG14361, M9831 and VXc-296.

95. The zinc-finger recombinase according to any one of paragraphs 8-25 for use in treating a disorder in a subject by modifying a target sequence in the genome of the cell.

96. The polynucleotide encoding a zinc-finger recombinase according to paragraph 26 or 27 for use in treating a disorder in a subject by modifying a target sequence in the genome of the cell.

97. The zinc-finger recombinase according to any one of paragraphs 8-25 for use in treating a disorder in a subject by excising a target sequence from the genome of the cell.

98. The polynucleotide encoding a zinc-finger recombinase according to paragraph 26 or 27 for use in treating a disorder in a subject, by excising a target sequence from the genome of the cell.

99. A zinc-finger recombinase for use in treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

100. A polynucleotide encoding a zinc-finger recombinase for use in treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

101. The zinc finger recombinase or polynucleotide encoding a zinc finger recombinase for use according to any one of paragraphs 97-100, for use with a non-homologous end joining (NHEJ) inhibitor.

102. The zinc finger recombinase or polynucleotide encoding a zinc finger recombinase for use according to paragraph 101, wherein the NHEJ inhibitor is selected from the group consisting of a small molecule inhibitor, a zinc-finger protein transcription factor (ZFP-TF), and a peptide inhibitor.

103. The zinc finger recombinase or polynucleotide encoding a zinc finger recombinase for use according to paragraph 102, wherein the small molecule inhibitor is selected from the group consisting of KU0060648, VX-984, W7, Chlorpromazine, Vanillin, Nu7026, Nu7441, Mirin, SCR7, AG14361, M9831 and VXc-296.

104. The zinc-finger recombinase according to any one of paragraphs 8-25 for use in correcting a disease-causing mutation in the genome of a cell by modifying a target sequence in the genome of the cell.

105. The polynucleotide encoding a zinc-finger recombinase according to paragraph 26 or 27 for use in correcting a disease-causing mutation in the genome of a cell by modifying a target sequence in the genome of the cell.

106. The zinc-finger recombinase according to any one of paragraphs 8-25 for use in correcting a disease-causing mutation in the genome of a cell by excising a target sequence from the genome of the cell.

107. The polynucleotide encoding a zinc-finger recombinase according to paragraph 26 or 27 for use in correcting a disease-causing mutation in the genome of a cell.

108. A zinc finger recombinase for use in correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

109. A polynucleotide encoding a zinc-finger recombinase for use in correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

110. The zinc finger recombinase or polynucleotide encoding a zinc finger recombinase for use according to any one of paragraphs 106-109, for use with a non-homologous end joining (NHEJ) inhibitor.

111. The zinc finger recombinase or polynucleotide encoding a zinc finger recombinase for use according to paragraph 110, wherein the NHEJ inhibitor is selected from the group consisting of a small molecule inhibitor, a zinc-finger protein transcription factor (ZFP-TF), and a peptide inhibitor.

112. The zinc finger recombinase or polynucleotide encoding a zinc finger recombinase for use according to paragraph 111, wherein the small molecule inhibitor is selected from a group consisting of KU0060648, VX-984, W7, Chlorpromazine, Vanillin, Nu7026, Nu7441, Mirin, SCR7, AG14361, M9831 and VXc-296.

113. The zinc finger recombinase or polynucleotide encoding a zinc finger recombinase for use according to any one of paragraphs 82-112, wherein the cell is a eukaryotic cell.

114. The zinc finger recombinase or polynucleotide encoding a zinc finger recombinase for use according to paragraph 113, wherein the cell is a mammalian cell.

115. The zinc finger recombinase or polynucleotide encoding a zinc finger recombinase for use according to paragraph 114, wherein the cell is a stem cell.

116. The zinc finger recombinase or polynucleotide encoding a zinc finger recombinase for use according to paragraph 113, wherein the cell is a human cell.

117. The zinc finger recombinase or polynucleotide encoding a zinc finger recombinase for use according to any one of paragraphs 82-116, wherein the use is independent of Fis.

118. The zinc finger recombinase or polynucleotide encoding a zinc finger recombinase for use according to any one of paragraphs 82-117, wherein the polynucleotide encoding the zinc-finger recombinase is introduced into the cell using a plasmid, a viral vector, a mini-circle or a linear DNA form.

119. The zinc finger recombinase or polynucleotide encoding a zinc finger recombinase for use according to any one of paragraphs 84-118, wherein the target nucleotide sequence is an endogenous locus.

120. The zinc finger recombinase or polynucleotide encoding a zinc finger recombinase for use according to paragraph 119, wherein the endogenous locus is selected from the group consisting of Hypoxanthine-guanine phosphoribosyltransferase (HPRT) gene, T Cell Receptor Alpha Constant (TRAC), Adeno-Associated Virus Integration Site 1 (AAVS1) and a safe-harbor locus.

121. Use of the zinc-finger recombinase according to any one of paragraphs 8-25 in the preparation of a medicament for modifying the genome of a cell.

122. Use of the polynucleotide encoding a zinc-finger recombinase according to paragraph 26 or 27 in the preparation of a medicament for modifying the genome of a cell.

123. Use of the zinc-finger recombinase according to any one of paragraphs 8-25 in the preparation of a medicament for integrating an exogenous nucleotide sequence into a target nucleotide sequence in the genome of a cell.

124. Use of the polynucleotide encoding a zinc-finger recombinase according to paragraph 26 or 27 in the preparation of a medicament for integrating an exogenous nucleotide sequence into a target nucleotide sequence in the genome of a cell.

125. Use of the zinc-finger recombinase according to any one of paragraphs 8-25 in the preparation of a medicament for disrupting a target nucleotide sequence in the genome of a cell.

126. Use of the polynucleotide encoding a zinc-finger recombinase according to paragraph 26 or 27 in the preparation of a medicament for disrupting a target nucleotide sequence in the genome of a cell.

127. Use of the zinc-finger recombinase according to any one of paragraphs 8-25 in the preparation of a medicament for excising a target nucleotide sequence from the genome of a cell.

128. Use of the polynucleotide encoding a zinc-finger recombinase according to paragraph 26 or 27 in the preparation of a medicament for excising a target nucleotide sequence from the genome of a cell.

129. Use of a zinc finger recombinase in the preparation of a medicament for excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

130. Use of a polynucleotide encoding a zinc-finger recombinase in the preparation of a medicament for excising a target nucleotide sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35.

131. The use according to any one of paragraphs 127-130, further comprising the use of a non-homologous end joining (NHEJ) inhibitor.

132. The use according to paragraph 131, wherein the NHEJ inhibitor is selected from the group consisting of a small molecule inhibitor, a zinc-finger protein transcription factor (ZFP-TF), and a peptide inhibitor.

133. The use according to paragraph 132, wherein the small molecule inhibitor is selected from the group consisting of KU0060648, VX-984, W7, Chlorpromazine, Vanillin, Nu7026, Nu7441, Mirin, SCR7, AG14361, M9831 and VXc-296.

134. Use of the zinc-finger recombinase according to any one of paragraphs 8-25 in the preparation of a medicament for treating a disorder in a subject by modifying a target sequence in the genome of the cell.

135. Use of the polynucleotide encoding a zinc-finger recombinase according to paragraph 26 or 27 in the preparation of a medicament for treating a disorder in a subject by modifying a target sequence in the genome of the cell.

136. Use of the zinc-finger recombinase according to any one of paragraphs 8-25 in the preparation of a medicament for treating a disorder in a subject by excising a target sequence from the genome of the cell.

137. Use of the polynucleotide encoding a zinc-finger recombinase according to paragraph 26 or 27 in the preparation of a medicament for treating a disorder in a subject, by excising a target sequence from the genome of the cell.

138. Use of a zinc-finger recombinase in the preparation of a medicament for treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

139. Use of a polynucleotide encoding a zinc-finger recombinase in the preparation of a medicament for treating a disorder in a subject, by excising a target sequence from the genome of a cell, wherein the zinc-finger recombinase comprises a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

140. The use according to any one of paragraphs 136-139, for use with a non-homologous end joining (NHEJ) inhibitor.

141. The use according to paragraph 140, wherein the NHEJ inhibitor is selected from the group consisting of a small molecule inhibitor, a zinc-finger protein transcription factor (ZFP-TF), and a peptide inhibitor.

142. The use according to paragraph 141, wherein the small molecule inhibitor is selected from the group consisting of KU0060648, VX-984, W7, Chlorpromazine, Vanillin, Nu7026, Nu7441, Mirin, SCR7, AG14361, M9831 and VXc-296.

143. Use of the zinc-finger recombinase according to any one of paragraphs 8-25 in the preparation of a medicament for correcting a disease-causing mutation in the genome of a cell by modifying a target sequence in the genome of the cell.

144. Use of a polynucleotide encoding a zinc-finger recombinase according to paragraph 26 or 27 in the preparation of a medicament for correcting a disease-causing mutation in the genome of a cell by modifying a target sequence in the genome of the cell.

145. Use of the zinc-finger recombinase according to any one of paragraphs 8-25 in the preparation of a medicament for correcting a disease-causing mutation in the genome of a cell by excising a target sequence from the genome of the cell.

146. Use of the polynucleotide encoding a zinc-finger recombinase according to paragraph 26 or 27 in the preparation of a medicament for correcting a disease-causing mutation in the genome of a cell.

147. Use of a zinc finger recombinase in the preparation of a medicament for correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

148. Use of a polynucleotide encoding a zinc-finger recombinase in the preparation of a medicament for correcting a disease-causing mutation in the genome of a cell by excising a target sequence from the genome of the cell, wherein the zinc-finger recombinase comprises a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

149. The use according to any one of paragraphs 145-148, for use with a non-homologous end joining (NHEJ) inhibitor.

150. The use according to paragraph 149, wherein the NHEJ inhibitor is selected from the group consisting of a small molecule inhibitor, a zinc-finger protein transcription factor (ZFP-TF), and a peptide inhibitor.

151. The use according to paragraph 150, wherein the small molecule inhibitor is selected from a group consisting of KU0060648, VX-984, W7, Chlorpromazine, Vanillin, Nu7026, Nu7441, Mirin, SCR7, AG14361, M9831 and VXc-296.

152. The use according to any one of paragraphs 121-151, wherein the cell is a eukaryotic cell.

153. The use according to paragraph 152, wherein the cell is a mammalian cell.

154. The use according to paragraph 153, wherein the cell is a stem cell.

155. The use according to paragraph 152, wherein the cell is a human cell.

156. The use according to any one of paragraphs 121-155, wherein the use is independent of Fis.

157. The use according to any one of paragraphs 121-156, wherein the polynucleotide encoding the zinc-finger recombinase is introduced into the cell using a plasmid, a viral vector, a mini-circle or a linear DNA form.

158. The use according to any one of paragraphs 123-157, wherein the target nucleotide sequence is an endogenous locus.

159. The use according to paragraph 158, wherein the endogenous locus is selected from the group consisting of Hypoxanthine-guanine phosphoribosyltransferase (HPRT) gene, T Cell Receptor Alpha Constant (TRAC), Adeno-Associated Virus Integration Site 1 (AAVS1) and a safe-harbor locus.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A Gin recombinase catalytic domain variant comprising a Phe104Asn amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

2. The Gin recombinase catalytic domain variant according to claim 1, further comprising a His106Tyr amino acid substitution.

3. The Gin recombinase catalytic domain variant according to claim 1, further comprising an Ile94Val amino acid substitution.

4. A polynucleotide encoding a Gin recombinase catalytic domain variant according to claim 1.

5. A polynucleotide encoding a Gin recombinase catalytic domain variant, wherein the nucleic acid sequence encoding the Gin recombinase catalytic domain variant comprises the nucleotide sequence set forth in SEQ ID NO: 7.

6. A zinc-finger recombinase, comprising a Gin recombinase catalytic domain variant operatively linked to a zinc-finger nucleotide binding domain, wherein the Gin recombinase catalytic domain variant comprises a Phe104Asn amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

7. The zinc finger recombinase according to claim 6, wherein the Gin recombinase catalytic domain variant further comprises a His106Tyr amino acid substitution.

8. The zinc finger recombinase according to claim 6, wherein the Gin recombinase catalytic domain variant further comprises an Ile94Val amino acid substitution.

9. The zinc-finger recombinase according to claim 6, wherein the zinc-finger recombinase protein is a multimeric protein.

10. The zinc-finger recombinase according to claim 6, wherein the zinc finger nucleotide binding domain comprises the sequence as set forth in SEQ ID NO: 9 or SEQ ID NO: 10.

11. The zinc-finger recombinase according to claim 6, wherein the zinc finger recombinase protein binds a nucleotide sequence comprising the sequence as set forth in SEQ ID NO: 15.

12. The zinc-finger recombinase according to claim 6, wherein the zinc finger nucleotide binding domain is capable of binding an endogenous locus.

13. The zinc-finger recombinase according to claim 12, wherein the endogenous locus is selected from the group consisting of Hypoxanthine-guanine phosphoribosyltransferase (HPRT) gene, T Cell Receptor Alpha Constant (TRAC) gene, Adeno-Associated Virus Integration Site 1 (AAVS1) and a safe-harbor locus.

14. A polynucleotide encoding the zinc-finger recombinase according to claim 6.

15. The polynucleotide according to claim 14, wherein the nucleic acid sequence encoding the Gin recombinase catalytic domain variant comprises the nucleotide sequence set forth in SEQ ID NO: 7.

16. A vector comprising the polynucleotide encoding the Gin recombinase catalytic domain variant according to claim 4.

17. A vector comprising the polynucleotide encoding a zinc-finger recombinase according to claim 14.

18. A cell comprising the vector according to claim 16 or 17.

19. A cell comprising the Gin recombinase catalytic domain variant according to claim 1.

20. A cell comprising the polynucleotide encoding the Gin recombinase catalytic domain variant according to claim 4.

21. A cell comprising the zinc finger recombinase protein according to claim 6.

22. A cell comprising the polynucleotide encoding the zinc-finger recombinase according to claim 14.

23. A pharmaceutical composition comprising the Gin recombinase catalytic domain variant according to claim 1; and a pharmaceutically acceptable carrier.

24. A pharmaceutical composition comprising the polynucleotide encoding the Gin recombinase catalytic domain variant according to claim 4; and a pharmaceutically acceptable carrier.

25. A pharmaceutical composition comprising the zinc-finger recombinase according to claim 6; and a pharmaceutically acceptable carrier.

26. A pharmaceutical composition comprising the polynucleotide encoding a zinc-finger recombinase according to claim 14; and a pharmaceutically acceptable carrier.

27. A method for modifying the genome of a cell, the method comprising introducing into a cell the zinc-finger recombinase according to claim 6.

28. A method for modifying the genome of a cell, the method comprising introducing into the cell the polynucleotide encoding a zinc-finger recombinase according to claim 14.

29. A method for integrating an exogenous nucleotide sequence into a target nucleotide sequence in the genome of a cell, the method comprising introducing into a cell the zinc-finger recombinase according to claim 6.

30. A method for integrating an exogenous nucleotide sequence into a target nucleotide sequence in the genome of a cell, the method comprising introducing into the cell the polynucleotide encoding a zinc-finger recombinase according to claim 14.

31. A method for disrupting a target nucleotide sequence in the genome of a cell, the method comprising introducing into the cell the zinc-finger recombinase according to claim 6.

32. A method for disrupting a target nucleotide sequence in the genome of a cell, the method comprising introducing into the cell the polynucleotide encoding a zinc-finger recombinase according to claim 14.

33. A method for excising a target nucleotide sequence from the genome of a cell, the method comprising introducing into the cell the zinc-finger recombinase according to claim 6.

34. A method for excising a target nucleotide sequence from the genome of a cell, the method comprising introducing into the cell the polynucleotide encoding a zinc-finger recombinase according to claim 14.

35. A method for excising a target nucleotide sequence from the genome of a cell, the method comprising introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

36. A method for excising a target nucleotide sequence from the genome of a cell, the method comprising introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of sequences SEQ ID NO: 1, SEQ ID NO: 31, SEQ ID NO: 32, SEQ ID NO: 33, SEQ ID NO: 34 or SEQ ID NO: 35.

37. The method according to any one of claims 35-36, further comprising introducing into the cell a non-homologous end joining (NHEJ) inhibitor.

38. A method for treating a disorder in a subject, the method comprising modifying a target sequence in the genome of the cell by introducing into the cell the zinc-finger recombinase according to claim 6.

39. A method for treating a disorder in a subject, the method comprising modifying a target sequence in the genome of the cell by introducing into the cell the polynucleotide encoding a zinc-finger recombinase according to claim 14.

40. A method for treating a disorder in a subject, the method comprising excising a target sequence from the genome of the cell by introducing into the cell the zinc-finger recombinase according to claim 6.

41. A method for treating a disorder in a subject, the method comprising excising a target sequence from the genome of the cell the polynucleotide encoding a zinc-finger recombinase according to claim 14.

42. A method for treating a disorder in a subject, the method comprising excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

43. A method for treating a disorder in a subject, the method comprising excising a target sequence from the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

44. The method according to any one of claims 42-43, further comprising administering a non-homologous end joining (NHEJ) inhibitor.

45. A method for correcting a disease-causing mutation in the genome of a cell, the method comprising modifying a target sequence in the genome of the cell comprising introducing into the cell the zinc-finger recombinase according to claim 6.

46. A method for correcting a disease-causing mutation in the genome of a cell, the method comprising modifying a target sequence in the genome of the cell comprising introducing into the cell the polynucleotide encoding a zinc-finger recombinase according to claim 14.

47. A method for correcting a disease-causing mutation in the genome of a cell, the method comprising excising a target sequence from the genome of the cell by introducing into the cell the zinc-finger recombinase according to claim 6.

48. A method for correcting a disease-causing mutation in the genome of a cell, the method comprising excising a target sequence in the genome of the cell by introducing into the cell the polynucleotide encoding a zinc-finger recombinase according to claim 14.

49. A method for correcting a disease-causing mutation in the genome of a cell, the method comprising excising a target sequence from the genome of the cell by introducing into the cell a zinc-finger recombinase comprising a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

50. A method for correcting a disease-causing mutation in the genome of a cell, the method comprising excising a target sequence in the genome of the cell by introducing into the cell a polynucleotide encoding a zinc-finger recombinase comprising a Gin recombinase catalytic domain variant operatively linked to a zinc-finger nucleotide binding domain, wherein the Gin recombinase catalytic domain variant further comprises a His106Tyr amino acid substitution with reference to a Gin recombinase catalytic domain amino acid sequence as set forth in any one of SEQ ID NO: 1, SEQ ID NO: 31, SEQ ID NO: 32, SEQ ID NO: 33, SEQ ID NO: 34 or SEQ ID NO: 35.

51. The method according to any one of claims 49-50, wherein the method further comprises administering a non-homologous end joining (NHEJ) inhibitor.

52. The method according to claim 45, wherein the method is independent of Fis.
